(19) United States
(12) Patent Application Publication
(10) Pub. No.: US 2005/0256159 A1
(43) Pub. Date: Nov. 17, 2005

(54) 1,4-disubstituted piperidine derivatives and their use as 11,betahsd1 inhibitors

(75) Inventors: Peter John Barton, Macclesfield (GB); Peter John Jewsbury, Macclesfield (GB); Janet Elizabeth Pease, Macclesfield (GB)

Correspondence Address: FISH & NEAVE IP GROUP;ROPES & GRAY LLP, ONE INTERNATIONAL PLACE, BOSTON, MA, 02110-2624, US

(73) Assignee: AstraZeneca AB, Sodertalje (SE)

(21) Appl. No.: 10/529,951

(22) PCT Filed: Oct. 7, 2003
(86) PCT No.: PCT/GB03/04318

§ 371 Date: Apr. 1, 2005

(30) Foreign Application Priority Data
Oct. 11, 2002 (GB) 02235737
May 7, 2003 (GB) 03104460

(51) Int. Cl. A61K 31/4709; A61K 31/452; C07D 45/02; C07D 41/02

(52) U.S. Cl. 514/314; 514/317; 514/326; 546/169; 546/207; 546/225

(57) ABSTRACT

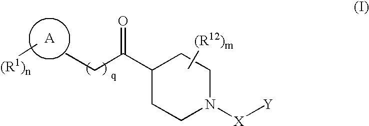

A method for inhibiting 11βHSD1 by administering a compound of formula (I) is described, wherein A, X, Y, R¹, R¹², n, q, and m are as described in the specification. Novel compounds and methods employing them are also described and claimed.

This invention relates to chemical compounds, or pharmaceutically acceptable salts thereof. These compounds possess human 11-β-hydroxysteroid dehydrogenase type 1 enzyme (11βHSD1) inhibitory activity and accordingly have value in the treatment of disease states including metabolic syndrome and are useful in methods of treatment of a warm-blooded animal, such as man. The invention also relates to processes for the manufacture of said compounds, to pharmaceutical compositions containing them and to their use in the manufacture of medicaments to inhibit 11βHSD1 in a warm-blooded animal, such as man.

Glucocorticoids (cortisol in man, corticosterone in rodents) are counter regulatory hormones i.e. they oppose the actions of insulin (Dalman M F, Strack A M, Akana S F et al. 1993; Front Neuroendocrinol 14, 303-347). They regulate the expression of hepatic enzymes involved in gluconeogenesis and increase substrate supply by releasing glycerol from adipose tissue (increased lipolysis) and amino acids from muscle (decreased protein synthesis and increased protein degradation). Glucocorticoids are also important in the differentiation of pre-adipocytes into mature adipocytes which are able to store triglycenrdes (Bujalska I J et al. 1999; Endocrinology 140, 3188-3196). This may be critical in disease states where glucocorticoids induced by “stress” are associated with central obesity which itself is a strong risk factor for type 2 diabetes, hypertension and cardiovascular disease (Bjorntorp P & Rosmond R 2000; Int. J. Obesity 24, S80-S85)

It is now well established that glucocorticoid activity is controlled not simply by secretion of cortisol but also at the tissue level by intracellular interconversion of active cortisol and inactive cortisone by the 11-beta hydroxysteroid dehydrogenases, 11βHSD1 (which activates cortisone) and 11βHSD2 (which inactivates cortisol) (Sandeep T C & Walker B R 2001 Trends in Endocrinol & Metab. 12, 446-453). That this mechanism may be important in man was initially shown using carbenoxolone (an anti-ulcer drug which inhibits both 11βHSD1 and 2) treatment which (Walker B R et al. 1995; J. Clin. Endocrinol. Metab. 80, 3155-3159) leads to increased insulin sensitivity indicating that 11βHSD1 may well be regulating the effects of insulin by decreasing tissue levels of active glucocorticoids (Walker B R et al. 1995; J. Clin. Endocrinol. Metab. 80, 3155-3159).

Clinically, Cushing's syndrome is associated with cortisol excess which in turn is associated with glucose intolerance, central obesity (caused by stimulation of pre-adipocyte differentiation in this depot), dyslipidaemia and hypertension. Cushing's syndrome shows a number of clear parallels with metabolic syndrome. Even though the metabolic syndrome is not generally associated with excess circulating cortisol levels (Jessop D S et al. 2001; J. Clin. Endocrinol. Metab. 86, 4109-4114) abnormally high 11βHSD1 activity within tissues would be expected to have the same effect. In obese men it was shown that despite having similar or lower plasma cortisol levels than lean controls, 11βHSD1 activity in subcutaneous fat was greatly enhanced (Rask E et al. 2001; J. Clin. Endocrinol. Metab. 1418-1421). Furthermore, the central fat, associated with the metabolic syndrome expresses much higher levels of 11βHSD1 activity than subcutaneous fat (Bujalska I J et al. 1997; Lancet 349, 1210-1213). Thus there appears to be a link between glucocorticoids, 11βHSD1 and the metabolic syndrome.

11βHSD1 knock-out mice show attenuated glucocorticoid-induced activation of gluconeogenic enzymes in response to fasting and lower plasma glucose levels in response to stress-or obesity (Kotelevtsev Y et al. 1997; Proc. Natl. Acad. Sci USA 94, 14924-14929) indicating the utility of inhibition of 11βHSD1 in lowering of plasma glucose and hepatic glucose output in type 2 diabetes. Furthermore, these mice express an anti-atherogenic lipoprotein profile, having low triglycerides, increased HDL cholesterol and increased apo-lipoprotein Al levels. (Morton N M et al. 2001; J. Biol. Chem. 276, 41293-41300). This phenotype is due to an increased hepatic expression of enzymes of fat catabolism and PPARα. Again this indicates the utility of 11βHSD1 inhibition in treatment of the dyslipidaemia of the metabolic syndrome.

The most convincing demonstration of a link between the metabolic syndrome and 11βHSD1 comes from recent studies of transgenic mice over-expressing 11βHSD1 (Masuzaki H et al. 2001; Science 294, 2166-2170). When expressed under the control of an adipose specific promoter, 11βHSD1 transgenic mice have high adipose levels of corticosterone, central obesity, insulin resistant diabetes, hyperlipidaemia and hyperphagia. Most importantly, the increased levels of 11βHSD1 activity in the fat of these mice are similar to those seen in obese subjects. Hepatic 11βHSD1 activity and plasma corticosterone levels were normal, however, hepatic portal vein levels of corticosterone were increased 3 fold and it is thought that this is the cause of the metabolic effects in liver.

Overall it is now clear that the complete metabolic syndrome can be mimicked in mice simply by overexpressing 11βHSD1 in fat alone at levels similar to those in obese man.

11βHSD1 tissue distribution is widespread and overlapping with that of the glucocorticoid receptor. Thus, 11HSD1 inhibition could potentially oppose the effects of glucocorticoids in a number of physiological/pathological roles. 11βHSD1 is present in human skeletal muscle and glucocorticoid opposition to the anabolic effects of insulin on protein turnover and glucose metabolism are well documented (Whorwood C B et al. 2001; J. Clin. Endocrinol. Metab. 86, 2296-2308). Skeletal muscle must therefore be an important target for 11βHSD1 based therapy.

Glucocorticoids also decrease insulin secretion and this could exacerbate the effects of glucocorticoid induced insulin resistance. Pancreatic islets express 11βHSD1 and carbenoxolone can inhibit the effects of 11-dehydocorticosterone on insulin release (Davani B et al. 2000; J. Biol. Chem. 275, 34841-34844). Thus in treatment of diabetes 11βHSD1 inhibitors may not only act at the tissue level on insulin resistance but also increase insulin secretion itself.

Skeletal development and bone function is also regulated by glucocorticoid action. 11βHSD1 is present in human bone osteoclasts and osteoblasts and treatment of healthy volunteers with carbenoxolone showed a decrease in bone resorption markers with no change in bone formation markers (Cooper M S et al 2000; Bone 27, 375-381). Inhibition of 11βHSD1 activity in bone could be used as a protective mechanism in treatment of osteoporosis.

Glucocorticoids may also be involved in diseases of the eye such as glaucoma. 11βHSD1 has been shown to affect intraocular pressure in man and inhibition of 11βHSD1 may be expected to alleviate the increased intraocular pressure associated with glaucoma (Rauz S et al. 2001; Investigative Opthalmology & Visual Science 42, 2037-2042).

There appears to be a convincing link between 11βHSD1 and the metabolic syndrome both in rodents and in humans. Evidence suggests that a drug which specifically inhibits 11βHSD1 in type 2 obese diabetic patients will lower blood glucose by reducing hepatic gluconeogenesis, reduce central obesity, improve the atherogenic lipoprotein phenotype, lower blood pressure and reduce insulin resistance. Insulin effects in muscle will be enhanced and insulin secretion from the beta cells of the islet may also be increased.

Currently there are two main recognised definitions of metabolic syndrome.

1) The Adult Treatment Panel (ATP III 2001 JMA) definition of metabolic syndrome indicates that it is present if the patient has three or more of the following symptoms:
Waist measuring at least 40 inches (102 cm) for men, 35 inches (88 cm) for women;
Serum triglyceride levels of at least 150 mg/dl (1.69 mmol/l);
HDL cholesterol levels of less than 40 mg/dl (1.04 mmol/l) in men, less than 50 mg/dl (1.29 mmol/l) in women;
Blood pressure of at least 135/80 mm Hg; and/or
Blood sugar (serum glucose) of at least 110 mg/dl (6.1 mmol/l).

2) The WHO consultation has recommended the following definition which does not imply causal relationships and is suggested as a working definition to be improved upon in due course:
The patient has at least one of the following conditions: glucose intolerance, impaired glucose tolerance (IGT) or diabetes mellitus and/or insulin resistance; together with two or more of the following:
Raised Arterial Pressure;
Raised plasma triglycerides
Central Obesity
Microalbuminuria

We have found that the compounds defined in the present invention, or a pharmaceutically acceptable salt thereof, are effective 11βHSD1 inhibitors, and accordingly have value in the treatment of disease states associated with metabolic syndrome.

Accordingly there is provided the use of a compound of formula (I): 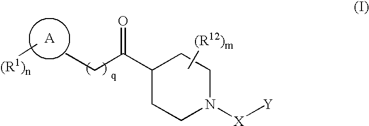
wherein:
Ring A is selected, from carbocyclyl or heterocyclyl; wherein if said heterocyclyl contains an —NH— moiety that nitrogen may be optionally substituted by a group selected from R⁹;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more groups selected from R³; and wherein if said heterocyclyl contains an —NH— moiety that nitrogen may be optionally substituted by a group selected from R⁴;
n is 0-5; wherein the values of R¹ may be the same or different;
X is a direct bond, —C(O)—, —S(O)₂—, —C(O)NR¹¹—, —C(S)NR¹¹—, —C(O)O—, —C(═NR¹¹)— or —CH₂—; wherein R¹¹ is selected from hydrogen, C₁₋₄alkyl, carbocyclyl and heterocyclyl;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸; and wherein if said heterocyclyl contains an —NH— moiety that nitrogen may be optionally substituted by a group selected from R¹³;
R⁴, R⁵, R⁷ R⁹ and R¹³are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;

R⁸ is selected from halo, nitro, cyano, hydroxy, trifluoromethoxy, trifluoromethyl, amino, carboxy, carbamoyl, mercapto,-sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
R¹² is hydroxy, methyl, ethyl or propyl;
m is 0 or 1;
q is 0 or 1;
   or a pharmaceutically acceptable salt thereof;
   in the manufacture of a medicament for use in the inhibition of 11βHSD1.

Accordingly to another feature of the invention, there is provided the use of a compound of formula (I′): 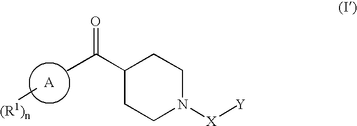
wherein:
Ring A is selected from aryl or heteroaryl; wherein if said heteroaryl contains an —NH— moiety that nitrogen may be optionally substituted by a group selected from R⁹;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; or two R¹ on adjacent carbons may form an oxyC₁₋₄alkoxy group; wherein R¹ may be optionally substituted on carbon by one or more groups selected from R³; and wherein if said heterocyclyl contains an —NH— moiety that nitrogen may be optionally substituted by a group selected from R⁴;
n is 0-3; wherein the values of R¹ may be the same or different;
X is —C(O)—, —S(O)₂— or —CH₂—;
Y is C₁₋₆alkyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;

R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Y— and heterocyclylC₀₋₄alkylene-Y—; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂4alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R³ and R⁶ may be independently optionally substituted on carbon by one or more R⁸;
R⁴, R⁵, R⁷ and R⁹ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
   or a pharmaceutically acceptable salt thereof;
   in the manufacture of a medicament for use in the inhibition of 11βHSD1.

Accordingly there is provided the use of a compound of formula (I″): 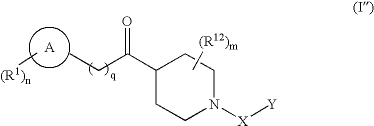
wherein:
Ring A is selected from carbocyclyl or heterocyclyl; wherein if said heterocyclyl contains an —NH— moiety that nitrogen may be optionally substituted by a group selected from R⁹;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more groups selected from R³; and wherein if said heterocyclyl contains an —NH— moiety that nitrogen may be optionally substituted by a group selected from R⁴;
n is 0-5; wherein the values of R¹ may be the same or different;
X is a direct bond, —C(O)—, —S(O)₂—, —C(O)NR¹¹—, —C(S)NR¹¹—, —C(O)O— or —CH₂—; wherein R¹¹ is selected from hydrogen and C₁₋₄-alkyl;
Y is hydrogen, C₁₋₆alkyl, C₂₋₆alkenyl, C₂₋₆alkynyl, carbocyclyl or heterocyclyl; wherein Y may be optionally substituted on carbon by one or more R², wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸;
R⁴, R⁵, R⁷ and R⁹ are independently selected from C₁₋₄alkyl, C₁₋₄-alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
R¹² is methyl or ethyl;
m is 0 or 1;
q is 0 or 1;
   or a pharmaceutically acceptable salt thereof;
   in the manufacture of a medicament for use in the inhibition of 11βHSD1.

In a further aspect of the invention, there is provided a compound of formula (Ia) wherein: 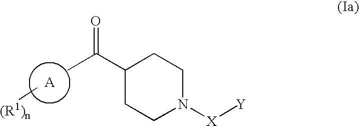
wherein:
Ring A is thienyl, furyl or thiazolyl;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄-alkyl)amino, N,N—(C₁₋₄alkyl)₂amino, C₁₋₄alkanoyl amino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; or two R¹ on adjacent carbons may form an oxyC₁₋₄alkoxy group; wherein R¹ may be optionally substituted on carbon by one or more groups selected from R³; and wherein if said heterocyclyl contains an —NH— moiety that nitrogen may be optionally substituted by a group selected from R⁴;
n is 0-3; wherein the values of R¹ may be the same or different;
X is —C(O)— or —S(O)₂—;
Y is C₁₋₆alkyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄-alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R³ and R⁶ may be independently optionally substituted on carbon by one or more R⁸;
R⁴, R⁵ and R⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
   or a pharmaceutically acceptable salt thereof;
   with the proviso that said compound is not
1-acetyl-4-[(4-methylthien-2-yl)carbonyl]piperidine;
1-acetyl-4-[(4-methyl-5-bromothien-2-yl)carbonyl]piperidine; or
1-benzoyl-4-[(5-methylthien-2-yl)carbonyl]piperidine.

In a further aspect of the invention, there is provided a compound of formula (Ib) wherein: 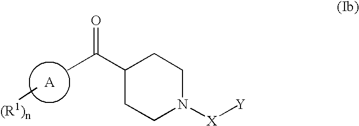
wherein:
Ring A is pyridinyl;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; or two R¹ on adjacent carbons may form an oxyC₁₋₄alkoxy group; wherein R¹ may be optionally substituted on carbon by one or more groups selected from R³; and wherein if said heterocyclyl contains an —NH— moiety that nitrogen may be optionally substituted by a group selected from R⁴;
n is 0-3; wherein the values of R¹ may be the same or different;
X is —C(O)— or —S(O)₂—;
Y is C₁₋₆alkyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄-alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R³ and R⁶ may be independently optionally substituted on carbon by one or more R⁸;
R⁴, R⁵ and R⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
   or a pharmaceutically acceptable salt thereof;
   with the proviso that said compound is not
1-(piperidin-4-ylcarbonyl)-4-(pyridin-2-ylcarbonyl)piperidine.

In a further aspect of the invention, there is provided a compound of formula (Ic): 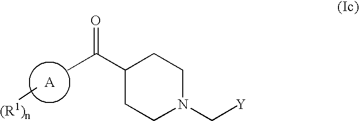
wherein:
Ring A is selected from thienyl, furyl, thiazolyl or pyridyl;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; or two R¹ on adjacent carbons may form an oxyC₁₋₄alkoxy group; wherein R¹ may be optionally substituted on carbon by one or more groups selected from R³; and wherein if said heterocyclyl contains an —NH— moiety that nitrogen may be optionally substituted by a group selected from R⁴;
n is 0-3; wherein the values of R¹ may be the same or different;
Y is phenyl, pyridyl, thienyl, furyl or thiazolyl; wherein Y may be optionally substituted on carbon by one or more R²;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R³ and R⁶ may be independently optionally substituted on carbon by one or more R⁸;
R⁴ and R⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino,-carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
   or a pharmaceutically acceptable salt thereof;
   with the proviso that said compound is not
1-(2-hydroxypyrid-3-ylmethyl)-4-(thien-2-ylcarbonyl)piperidine;
1-(2-methoxypyrid-3-ylmethyl)-4-(thien-2-ylcarbonyl)piperidine or
1-benzyl-4-(thien-2-ylcarbonyl)piperidine.

In a further feature of the invention, there is provided a compound of formula (Id): 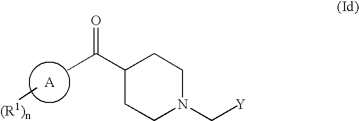
wherein:
Ring A is phenyl;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; or two R¹ on adjacent carbons may form an oxyC₁₋₄alkoxy group; wherein R¹ may be optionally substituted on carbon by one or more groups selected from R³; and wherein if said heterocyclyl contains an —NH— moiety that nitrogen may be optionally substituted by a group selected from R⁴;
n is 0-3; wherein the values of R¹ may be the same or different;
Y is thienyl, furyl or thiazolyl; wherein Y may be optionally substituted on carbon by one or more R²;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄-alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl and heterocyclyl; wherein R³ and R⁶ may be independently optionally substituted on carbon by one or more R⁸;
R⁴ and R⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
   or a pharmaceutically acceptable salt thereof;
   with the proviso that said compound is not
1-(thien-2-ylmethyl)-4-(4-mesylaminobenzoyl)piperidine or
1-(5-methylfur-2-ylmethyl)-4-(4-mesylaminobenzoyl)piperidine.

In a further aspect of the invention there is provided a compound of formula (Ie): 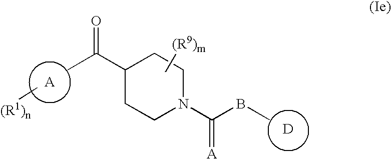
wherein:
Ring A is selected from carbon linked pyridyl, thienyl, furyl and thiazolyl;
A is O or S;
B is O or N;
Ring D is carbocyclyl or heterocyclyl; wherein Ring D may be optionally substituted on carbon by one or more R²; wherein if said heterocyclyl contains an —NH— moiety that nitrogen may be optionally substituted by a group selected from R⁵;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more groups selected from R³; and wherein if said heterocyclyl contains an —NH— moiety that nitrogen may be optionally substituted by a group selected from R⁴;
n is 0-5; wherein the values of R¹ may be the same or different;
X is a direct bond, —C(O)—, —S(O)₂—, —C(O)NR¹¹—, —C(S)NR¹¹—, —C(O)O— or —CH₂—; wherein R¹¹ is selected from hydrogen and C₁₋₄alkyl;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoroethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄-alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸;
R⁴, R⁵ and R⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
R¹² is methyl or ethyl;
m is 0 or 1;
q is 0 or 1;
   or a pharmaceutically acceptable salt thereof;
   with the proviso that said compound is not
1-(2-cyano-4,5-dimethoxyanilinothiocarbonyl)-4-(thien-2-ylcarbonyl)piperidine.

In a further aspect of the invention there is provided a compound of formula (If): 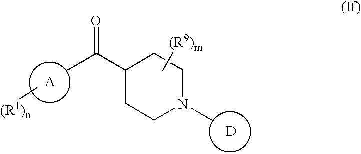
wherein:
Ring A is selected from carbon linked pyridyl, thienyl, furyl and thiazolyl;
Ring D is carbon linked phenyl, pyridyl, thienyl, furyl and thiazolyl; wherein Ring D may be optionally substituted on carbon by one or more R²; wherein said thiazolyl may be optionally substituted on nitrogen by a group selected from R⁵;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more groups selected from R³; and wherein if said heterocyclyl contains an —NH— moiety that nitrogen may be optionally substituted by a group selected from R⁴;
n is 0-5; wherein the values of R¹ may be the same or different;
X is a direct bond, —C(O)—, —S(O)₂—, —C(O)NR¹¹—, —C(S)NR¹¹—, —C(O)O— or —CH₂—; wherein R¹¹ is selected from hydrogen and C₁₋₄alkyl;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸;
R⁴, R⁵ and R⁷ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
R¹² is methyl or ethyl;
m is 0 or 1;
q is 0 or 1;
   or a pharmaceutically acceptable salt thereof.

According to a further aspect of the invention there is provided a compound of formula (Ig): 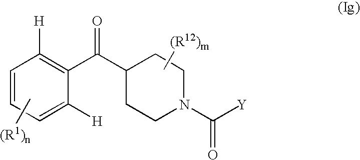
wherein:
R¹ is a substituent on carbon and is selected from halo, cyano, C₁₋₄alkyl, C₁₋₄alkoxy, C₁₋₄alkylS(O)₂, N—(C₁₋₄alkyl)sulphamoyl or N,N—(C₁₋₄alkyl)₂sulphamoyl; wherein R¹ may be optionally substituted on carbon by one or more groups selected from R³;
n is 0-3; wherein the values of R¹ may be the same or different;
Y is phenyl, pyrimidine, furan, thiophene or thiazole; wherein Y may be optionally substituted on carbon by one or more R²;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄-alkynyl, C₁₋₄alkoxy, C₁₋₄-alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄-alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio or N,N—(C₁₋₄alkyl)₂aminothiocarbonylthio; wherein R² may be optionally substituted on carbon by one or more groups selected from R⁶;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄-alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl or C₁₋₄alkylsulphonylamino; wherein R³ and R⁶ may be independently optionally substituted on carbon by one or more R⁸;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is-selected from hydrogen and C₁₋₄alkyl;
R¹² is hydroxy, methyl, ethyl or propyl;
m is 0 or 1;
   or a pharmaceutically acceptable salt thereof;
   with the proviso that said compound is not 1,4-dibenzoylpiperidine;
4-hydroxy-1,4-dibenzoylpiperidine; 1-(3,4,5-trimethoxybenzoyl)-1-benzoylpiperidine;
1,4-di-(4-methylbenzoyl)piperidine; 1-(4-chlorobenzoyl)4-benzoylpiperidine;
1-(3-nitrobenzoyl)-4-benzoylpiperidine;
1-(2-methoxy-4,6-ditrifluoromethylbenzoyl)-4-(4-chlorobenzoyl)piperidine;
1-(2,6-difluorobenzoyl)-4-benzoylpiperidine;
1-(3-trifluoromethylbenzoyl)-4-(benzoyl)piperidine;
1-(4-aminobenzoyl)-4-(4-fluorobenzoyl)piperidine;
1-(2-chloro-4-nitrobenzoyl)-4-benzoylpiperidine; 1-(4-methoxybenzoyl)4-benzoylpiperidine;
1-(4-t-butylbenzoyl)-4-benzoylpiperidine;
1-(2,4-dihydroxybenzoyl)-4-(4-fluorobenzoyl)piperidine;
1-(4-nitrobenzoyl)-4-(4-fluorobenzoyl)piperidine;
1-(pyrid-3-ylcarbonyl)-4-(4-fluorobenzoyl)piperidine;
1-(thien-2-ylcarbonyl)-4-benzoylpiperidine;
1-(thien-2-ylcarbonyl)-4-(4-methylbenzoyl)piperidine; or
1-(fur-2-ylcarbonyl)-4-benzoylpiperidine.

According to a further aspect of the invention there is provided the use of a compound of formula (Ih): 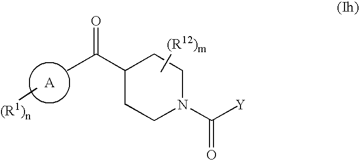
wherein:
Ring A is selected from carbocyclyl or heterocyclyl; wherein if said heterocyclyl contains an —NH— moiety that nitrogen may be optionally substituted by a group selected from R⁹;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more groups selected from R³; and wherein if said heterocyclyl contains an —NH— moiety that nitrogen may be optionally substituted by a group selected from R⁴;
n is 0-5; wherein the values of R¹ may be the same or different;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶; and wherein if said heterocyclyl contains an —NH— moiety that nitrogen may be optionally substituted by a group selected from R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄-alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸; and wherein if said heterocyclyl contains an —NH— moiety that nitrogen may be optionally substituted by a group selected from R¹³;
R⁴, R⁵, R⁷ R⁹ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
R¹² is hydroxy, methyl, ethyl or propyl;
m is 0 or 1;
   or a pharmaceutically acceptable salt thereof;
   in the manufacture of a medicament for use in the inhibition of 11βHSD1.

For the avoidance of doubt, where X is —C(O)NR¹¹—, —C(S)NR¹¹— or —C(O)O— is it the C(O) or the C(S) that is attached to the nitrogen of the piperidine ring in formula (I).

Also for the avoidance of doubt, where the use etc of compounds of formula (I) is referred to herein, it is to be understood that this also refers to the use of compounds of formula (I′) and (I″) as well.

In this specification the term “alkyl” includes both straight and branched chain alkyl groups but references to individual alkyl groups such as “propyl” are specific for the straight chain version only. For example, “C₁₋₆alkyl” and “C₁₋₄alkyl” includes propyl, isopropyl and t-butyl. However, references to individual alkyl groups such as ‘propyl’ are specific for the straight chained version only and references to individual branched chain alkyl groups such as ‘isopropyl’ are specific for the branched chain version only. A similar convention applies to other radicals therefore “carbocyclylC₁₋₄alkyl” would include 1-carbocyclylpropyl, 2-carbocyclylethyl and 3-carbocyclylbutyl. The term “halo” refers to fluoro, chloro, bromo and iodo.

Where optional substituents are chosen from “one or more” groups it is to be understood that this definition includes all substituents being chosen from one of the specified groups or the substituents being chosen from two or more of the specified groups.

“Heteroaryl” is a totally unsaturated, mono or bicyclic ring containing 3-12 atoms of which at least one atom is chosen from nitrogen, sulphur or oxygen, which may, unless otherwise specified, be carbon or nitrogen linked. Suitably “heteroaryl” refers to a totally unsaturated, monocyclic ring containing 5 or 6 atoms or a bicyclic ring containing 8-10 atoms of which at least one atom is chosen from nitrogen, sulphur or oxygen, which may, unless otherwise specified, be carbon or nitrogen linked. Examples and suitable values of the term “heteroaryl” are thienyl, furyl, thiazolyl, pyrazolyl, isoxazolyl, imidazolyl, pyrrolyl, thiadiazolyl, isothiazolyl, triazolyl, pyranyl, indolyl, pyrimidyl, pyrazinyl, pyridazinyl, benzothienyl, pyridyl and quinolyl. Particularly “heteroaryl” refers to thienyl, furyl, thiazolyl, pyridyl, benzothienyl, imidazolyl or pyrazolyl.

“Aryl” is a totally unsaturated, mono or bicyclic carbon ring that contains. 3-12 atoms. Suitably “aryl” is a monocyclic ring containing 5 or 6 atoms or a bicyclic ring containing 9 or 10 atoms. Suitable values for “aryl” include phenyl or naphthyl. Particularly “aryl” is phenyl.

A “heterocyclyl” is a saturated, partially saturated or unsaturated, mono, bicyclic or tricyclic ring containing 3-15 atoms of which at least one atom is chosen from nitrogen, sulphur or oxygen, which may, unless otherwise specified, be carbon or nitrogen linked, wherein a —CH₂— group can optionally be replaced by a —C(O)— or a —C(S)—, or a ring sulphur atom may be optionally oxidised to form the S-oxides. Particularly a “heterocyclyl” is a saturated, partially saturated or unsaturated, mono or bicyclic ring containing 3-12 atoms of which at least one atom is chosen from nitrogen, sulphur or oxygen, which may, unless otherwise specified, be carbon or nitrogen linked, wherein a —CH₂— group can optionally be replaced by a —C(O)— or a —C(S)—, or a ring sulphur atom may be optionally oxidised to form the S-oxides. More particularly a “heterocyclyl” is a saturated, partially saturated or unsaturated, mono or bicyclic ring containing 3-12 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form the S-oxides. Preferably a “heterocyclyl” is a saturated, partially saturated or unsaturated, mono or bicyclic ring containing 5 or 6 atoms of which at least one atom is chosen from nitrogen, sulphur or oxygen, which may, unless otherwise specified, be carbon or nitrogen linked, wherein a —CH₂— group can optionally be replaced by a —C(O)— or a ring sulphur atom may be optionally oxidised to form S-oxide(s). Examples and suitable values of the term “heterocyclyl” are thienyl, piperidinyl, morpholinyl, furyl, thiazolyl, pyridyl, imidazolyl, 1,2,4-triazolyl, thiomorpholinyl, coumarinyl, pyrimidinyl, phthalidyl, pyrazolyl, pyrazinyl, pyridazinyl, benzothienyl, benzimidazolyl, tetrahydrofuryl, [1,2,4]triazolo[4,3-a]pyrimidinyl, piperidinyl, indolyl, 1,3-benzodioyolyl and pyrrolidinyl. Further examples and suitable values of the term “heterocyclyl” are 1,3-benzodioxolyl, thienyl, furyl, thiazolyl, pyrazinyl, pyrrolyl, indolyl, quinolinyl, isoquinolinyl, pyrazolyl, isoxazolyl, benzofuranyl, 1,2,3-thiadiazolyl, 1,2,5-thiadiazolyl, pyrimidinyl, 2,1-benzisoxazolyl, 4,5,6,7-tetrahydro-2H-indazolyl, imidazo[2,1-b][1,3]thiazolyl, tetrahydrofuranyl, tetrahydropyranyl, piperidinyl, morpholinyl, 2,3-dihydro-1-benzofuryl, 2,3-dihydro-1,4-benzodioxinyl and pyridyl. Further examples and suitable values for the term “heterocyclyl” are benzofuranyl, 2,1-benzisoxazolyl, 1,3-benzodioxolyl, 1,3-benzothiazolyl, benzothienyl, 3,4-dihydro-2H-benzodioxepinyl, 2,3-dihydro-1,4-benzodioxinyl, chromanyl, 2,3-dihydrobenzofuranyl, furyl, imidazo[2,1-b][1,3]thiazolyl, indolyl, isoindolinyl, isoquinolinyl, isoxazolyl, morpholinyl, oxazolyl, piperidinyl, pyrazinyl, pyrazolyl, pyridyl, pyridazinyl, pyrimidinyl, pyrrolidinyl, pyrrolyl, quinolinyl, quinoxalinyl, tetrahydrofuryl, 4,5,6,7-tetrahydro-1-benzofuryl, 4,5,6,7-tetrahydro-2H-indazolyl, 4,5,6,7-tetrahydro-1H-indolyl, tetrahydropyranyl, 1,2,3,4tetrahydroquinolinyl, thiazolyl, 1,2,3-thiadiazolyl, 1,2,5-thiadiazolyl or thienyl.

A “carbocyclyl” is a saturated, partially saturated or unsaturated, mono, bicyclic or tricyclic carbon ring that contains 3-15 atoms; wherein a —CH₂— group can optionally be replaced by a —C(O)—. Particularly a “carbocyclyl” is a saturated, partially saturated or unsaturated, mono or bicyclic carbon ring that contains 3-12 atoms; wherein a —CH₂— group can optionally be replaced by a —C(O)—. Preferably “carbocyclyl” is a monocyclic ring containing 5 or 6 atoms or a bicyclic ring containing 9 or 10 atoms. Suitable values for “carbocyclyl” include cyclopropyl, cyclobutyl, 1-oxocyclopentyl, cyclopentyl, cyclopentenyl, cyclohexyl, cyclohexenyl, phenyl, naphthyl, tetralinyl, indanyl or 1-oxoindanyl. Particularly “carbocyclyl” is cyclohexyl, phenyl, naphthyl or 2-6-dioxocyclohexyl. More particularly “carbocyclyl” is phenyl, naphthyl, cyclopropyl, cyclopentyl, cyclohexyl, 1,2,3,4-tetrahydronaphthyl or indenyl. More particularly “carbocyclyl” is naphthyl, phenyl, cyclopropyl, cyclohexyl, indenyl, 1,2,3,4-tetrahydronaphthyl, cyclopentyl or (3r)-adamantanyl.

An example of “C₁₋₄alkanoyloxy” is acetoxy. Examples of “C₁₋₄alkoxycarbonyl” include methoxycarbonyl, ethoxycarbonyl, n- and t-butoxycarbonyl. Examples of “C₁₋₄alkoxy” include methoxy, ethoxy and propoxy. Examples of “oxyC₁₋₄alkoxy” include oxymethoxy, oxyethoxy and oxypropoxy. Examples of “C₁₋₄alkanoylamino” include formamido, acetamido and propionylamino. Examples of and “C₁₋₄alkylS(O)ₐ wherein a is 0 to 2” include methylthio, ethylthio, methylsulphinyl, ethylsulphinyl, mesyl and ethylsulphonyl. Examples of and “C₁₋₄alkylsulphonyl” include mesyl and ethylsulphonyl. Examples of “C₁₋₄alkanoyl” include propionyl and acetyl. Examples of “N—(C₁₋₄alkyl)amino” include methylamino and ethylamino. Examples of “N,N—(C₁₋₄alkyl)₂amino” include di-N-methylamino, di-(N-ethyl)amino and N-ethyl-N-methylamino. Examples of “C₂₋₄alkenyl” are vinyl, allyl and 1-propenyl. Examples of “C₂₋₄alkynyl” are ethynyl, 1-propynyl and 2-propynyl. Examples of “N—(C₁₋₄alkyl)sulphamoyl” are N-(methyl)sulphamoyl and N-(ethyl)sulphamoyl. Examples of “N—(C₁₋₄alkyl)₂sulphamoyl” are N,N-(dimethyl)sulphamoyl and N-(methyl)-N-(ethyl)sulphamoyl. Examples of “N—(C₁₋₄alkyl)carbamoyl” are methylaminocarbonyl and ethylaminocarbonyl. Examples of “N,N—(C₁₋₄alkyl)₂carbamoyl” are dimethylaminocarbonyl and methylethylaminocarbonyl. Examples of “C₁₋₄alkylsulphonylamino” are mesylamino and ethylsulphonylamino. Examples of “C₀₋₄alkylene” are a direct bond, methylene and ethylene.

A suitable pharmaceutically acceptable salt of a compound of the invention is, for example, an acid-addition salt of a compound of the invention which is sufficiently basic, for example, an acid-addition salt with, for example, an inorganic or organic acid, for example hydrochloric, hydrobromic, sulphuric, phosphoric, trifluoroacetic, citric or maleic acid. In addition a suitable pharmaceutically acceptable salt of a compound of the invention which is sufficiently acidic is an alkali metal salt, for example a sodium or potassium salt, an alkaline earth metal salt, for example a calcium or magnesium salt, an ammonium salt or a salt with an organic base which affords a physiologically-acceptable cation, for example a salt with methylamine, dimethylamine, trimethylamine, piperidine, morpholine or tris-(2-hydroxyethyl)amine.

Some compounds of the formula (I) may have chiral centres and/or geometric isomeric centres (E- and Z-isomers), and it is to be understood that the invention encompasses all such optical, diastereoisomers and geometric isomers that possess 11βHSD1 inhibitory activity.

The invention relates to any and all tautomeric forms of the compounds of the formula (I) that possess 11βHSD1 inhibitory activity.

It is also to be understood that certain compounds of the formula (I) can exist in solvated as well as unsolvated forms such as, for example, hydrated forms. It is to be understood that the invention encompasses all such solvated forms which possess 11βHSD1 inhibitory activity.

Particular values of variable groups are as follows. Such values may be used where appropriate with any of the definitions, claims or embodiments defined hereinbefore or hereinafter.
Ring A is aryl.
Ring A is heteroaryl; wherein if said heteroaryl contains an —NH— moiety that nitrogen may be optionally substituted by a group selected from R⁹.
Ring A is aryl or heteroaryl; wherein if said heteroaryl contains an —NH— moiety that nitrogen may be optionally substituted by a group selected from R⁹.
Ring A is carbocyclyl.
Ring A is heterocyclyl; wherein if said heterocyclyl contains an —NH— moiety that nitrogen may be optionally substituted by a group selected from R⁹.
Ring A is phenyl.
Ring A is selected from phenyl, 1,3-benzodioxolyl, thienyl, cyclopentyl, pyridyl or furyl.
Ring A is phenyl, 1,3-benzodioxolyl, thienyl, cyclopentyl, pyridyl, furyl, thiazolyl, 1,3-benzothiazolyl, benzofuryl or benzothienyl.
Ring A is selected from phenyl, 1,3-benzodioxol-5-yl, thien-2-yl, cyclopentyl, pyrid-2-yl or fur-2-yl.
Ring A is phenyl wherein the positions ortho to the (CH₂)_{q} group are unsubstituted or substituted by fluoro, preferably unsubstituted.
R¹ is selected from halo or C₁₋₄alkyl.
R¹ is a substituent on carbon and is selected from halo, C₁₋₄alkyl, C₁₋₄alkoxy, carbocyclyl and carbocyclylC₀₋₄alkylene-Z-; wherein R¹ may be optionally substituted on carbon by one or more groups selected from R³; wherein R³ is halo; and Z is —S(O)ₐ—; wherein a is 2.
R¹ is a substituent on carbon and is selected from halo, cyano, C₁₋₄alkyl, C₁₋₄alkoxy, N,N—(C₁₋₄alkyl)₂amino, C₁₋₄alkylS(O)ₐ wherein a is 0 to 2, carbocyclyl and carbocyclylC₀₋₄alkylene-Z-; wherein R¹ may be optionally substituted on carbon by one or more groups selected from R³; wherein
R³ is selected from halo, hydroxy, C₁₋₄alkoxy, heterocyclyl and carbocyclylC₀₋₄alkylene-Z-; and
Z is —S(O)ₐ— or —O—; wherein a is 0 to 2.
R¹ is selected from fluoro, chloro or methyl.
R¹ is selected from fluoro, chloro, methoxy or methyl.
R¹ is a substituent on carbon and is selected from fluoro, chloro, bromo, methyl, t-butyl, propyl, methoxy, phenyl or 6-bromonaphth-2-ylsulphonyl.
R¹ is a substituent on carbon and is selected from fluoro, chloro, bromo, cyano, methyl, propyl, t-butyl, methoxy, ethoxy, isopropoxy, butoxy, naphth-2-ylthio, naphth-2-ylsulphonyl, phenyl, methylthio, isopropylthio, mesyl, isopropylsulphonyl, methylsulphinyl, isopropylsulphinyl and dimethylamino; wherein R¹ may be optionally substituted on carbon by one or more groups selected from R³; wherein
R³ is selected from fluoro, bromo, hydroxy, methoxy, benzyloxy and thienyl; and
Z is —S(O)ₐ—; wherein a is 0 to 2.
n is 0-3; wherein the values of R¹ may be the same or different.
n is 0-2; wherein the values of R¹ may be the same or different.
n is 0 or 1.
n is 2; wherein the values of R¹ may be the same or different.
n is 1.
n is 0.
Ring A is phenyl, n is 1 and the substituent is para to the carbonyl of formula (I).
Ring A, R¹ and n together form phenyl, 2-fluorophenyl, 3-fluorophenyl, 4-fluorophenyl, 3-chlorophenyl, 4-chlorophenyl, 4-bromophenyl, 2-methylphenyl, 3-methylphenyl, 4-methylphenyl, 4-propylphenyl, 4-t-butylphenyl, 2-methoxyphenyl, 3-methoxyphenyl, 4-methoxyphenyl, 4-(6-bromonaphth-2-ylsulphonyl)phenyl, 4-phenylphenyl, 2,4-difluorophenyl, 3,5-difluorophenyl, 2-methyl4-fluorophenyl, 2,4-dimethylphenyl, 1,3-benzodioxol-5-yl, thien-2-yl, 5-chlorothien-2-yl, cyclopentyl, pyrid-2-yl, 6-methylpyrid-2-yl and fur-2-yl.
Ring A, (R¹)ₙ and (CH²)_{q} together form phenyl, 4-bromophenyl, 3-butoxyphenyl, 4-t-butylphenyl, 3-chlorophenyl, 4-chlorophenyl, 3-cyanophenyl, 4-cyanophenyl, 4-dimethylaminophenyl, 3-ethoxyphenyl, 2-fluorophenyl, 3-fluorophenyl, 4-fluorophenyl, 3-isopropoxyphenyl, 4-isopropoxyphenyl, 4-(isopropylthio)phenyl, 4-(isopropylsulphinyl)phenyl, 4-(isopropylsulphonyl)phenyl, 3-mesylphenyl, 4-mesylphenyl, 3-(methoxymethyl)phenyl, 2-methoxyphenyl, 3-methoxyphenyl, 4-methoxyphenyl, 2-methylphenyl, 3-methylphenyl, 4-methylphenyl, 3-methylsulphinylphenyl, 4-methylsulphinylphenyl, 3-methylthiophenyl, 4-methylthiophenyl, 4-propylphenyl, 3-trifluoromethylphenyl, 4-trifluoromethylphenyl, 3-trifluoromethoxyphenyl, 4-trifluoromethoxyphenyl, 2,4-difluorophenyl, 3,5-difluorophenyl, 3,5-dichlorophenyl, 3,4-dichlorophenyl, 2,4-dimethylphenyl, 2-methyl-4-fluorophenyl, 3-methyl-4-chlorophenyl, 3-methyl4-methoxyphenyl, 3-chloro-4-fluorophenyl, 3-(benzyloxymethyl)4-chlorophenyl, 3-(hydroxymethyl)-4-chlorophenyl, 3-methoxy-4-chlorophenyl, 3-ethoxy4-chlorophenyl, 4-(6-bromonaphth-2-ylthio)phenyl, 4-(6-bromonaphth-2-ylsulphonyl)phenyl, benzyl, cyclopentyl, biphenyl-4-yl, 1,3-benzodioxol-5-yl, thien-2-yl, 4-chlorothien-2-yl, 5-chlorothien-2-yl 5-methylthien-2-yl, thien-3-yl, 6-methylpyrid-2-yl, pyrid-2-yl, fur-2-yl, 5-cyanofur-2-yl, 4,5-dimethylfur-2-yl, thiazol-2-yl, 4,5-dimethylthiazol-2-yl, 1,3-benzothiazol-2-yl, benzofur-2-yl, 5-chlorobenzofur-2-yl, benzothien-2-yl, 5-chlorobenzothien-2-yl, 5-(thien-2-yl)thien-2-yl,
Ring A, R¹ and n together form 4-fluorophenyl, 4-chlorophenyl and 4-methoxyphenyl.
X is —C(O)—.
X is —S(O)₂—.
X is —CH₂—.
X is —C(O)NR¹¹—; wherein R¹¹ is selected from hydrogen.
X is —C(O)NR¹¹—; wherein R¹¹ is selected from C₁₋₄alkyl.
X is —C(O)NR¹¹—; wherein R¹¹ is selected from methyl.
X is —C(S)NR¹¹—; wherein R¹¹ is selected from hydrogen.
X is —C(S)NR¹¹—; wherein R¹¹ is selected from C₁₋₄alkyl.
X is —C(O)O—.
X is a direct bond.
X is —C(═NR¹¹)—; wherein R¹¹ is selected from hydrogen.
X is —C(═NR¹¹)—; wherein R¹¹ is selected from C₁₋₄alkyl.
Y is C₁₋₆alkyl; wherein Y may be optionally substituted on carbon by one or more R².
Y is carbocyclyl; wherein Y may be optionally substituted on carbon by one or more R².
Y is heterocyclyl; wherein Y may be optionally substituted on carbon by one or more R²; wherein if said heterocyclyl contains an —NH— moiety that nitrogen may be optionally substituted by a group selected from R⁵.
Y is phenyl, thienyl, methyl, furyl, cyclopropyl or cyclohexyl; wherein Y may be optionally substituted on carbon by one or more R².
Y is phenyl, thien-2-yl, methyl, fur-2-yl, cyclopropyl or cyclohexyl; wherein Y may be optionally substituted on carbon by one or more R².
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵.
Y is hydrogen, methyl, ethyl, propyl, isopropyl, butyl, t-butyl, pentyl, naphthyl, phenyl, pyridyl, thienyl, furyl, cyclopropyl, cyclohexyl, thiazolyl, pyrazinyl, pyrrolyl, indolyl, quinolinyl, pyrazolyl, isoxazolyl, isoquinolinyl, indenyl, 1,2,3,4-tetrahydronaphthyl, benzofuranyl, 1,2,3-thiadiazolyl, 1,2,5-thiadiazolyl, pyrimidinyl, morpholinyl, piperidinyl, 2,1-benzisoxazolyl, 4,5,6,7-tetrahydro-2H-indazolyl, isoindolinyl, tetrahydrofuryl, imidazo[2,1-b][1,3]thiazolyl, cyclopentyl, 2,3-dihydro-1,4-benzodioxinyl, tetrahydropyranyl, 2,3-dihydrobenzofuranyl, 1,3-benzodioxolyl, benzothienyl, chromanyl, 1,2,3,4-tetrahydroquinolinyl, 1,3-benzothiazolyl, 3,4-dihydro-2H-benzodioxepinyl, (3r)-adamantanyl, pyrrolidinyl, oxazolyl, 4,5,6,7-tetrahydro-1H-indolyl, quinoxalinyl or 4,5,6,7-tetrahydro-1-benzofuryl; wherein Y may be optionally substituted on carbon by one or more R²; wherein if said heterocyclyl contains an —NH— moiety that nitrogen may be optionally substituted by a group selected from R⁵.
Y is 4-methylphenyl, 4-fluorophenyl, thien-2-yl, methyl, fur-2-yl, cyclopropyl or cyclohexyl; wherein Y may be optionally substituted on carbon by one or more R².
R² is a substituent on carbon and is selected from halo or C₁₋₄alkyl.
R² is a substituent on carbon and is selected from fluoro or methyl.
R² is a substituent on carbon and is selected from halo, nitro, cyano, amino, trifluoromethyl, trifluoromethoxy, C₁₋₄alkyl, C₁₋₄alkoxy, C₁₋₄alkanoyl, N—(C₁₋₄alkyl)amino, N,N—(C₁₋₄alkyl)₂amino, C₁₋₄alkanoylamino, C₁₋₄alkylS(O)ₐ wherein a is 0 or 2, C₁₋₄alkoxycarbonylamino, C₁₋₄alkoxycarbonyl-N—(C₁₋₄alkyl)amino, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶.
R⁶ is selected from halo, nitro, C₁₋₄alkyl, C₂₋₄alkenyl, C₁₋₄alkoxy, C₁₋₄alkoxycarbonylamino, carbocyclyl and carbocyclylC₀₋₄alkylene-Z-, wherein R⁶ may be optionally substituted on carbon by one or more R⁸;
R⁵ is selected from C₁₋₄alkyl and C₁₋₄alkoxycarbonyl.
R⁸ is selected from halo.
Z is —S(O)ₐ—, —O—, —C(O)— or —OC(O)NR¹⁰—; wherein a is 0 or 2; wherein R¹⁰ is selected from hydrogen.
When Y is phenyl, R² is para to X.
Y is hydrogen, C₁₋₄alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵; wherein
R² is a substituent on carbon and is selected from halo, nitro, cyano, amino, trifluoromethyl, trifluoromethoxy, C₁₋₄alkyl, C₁₋₄alkoxy, C₁₋₄alkanoyl, N—(C₁₋₄alkyl)amino, N,N—(C₁₋₄alkyl)₂amino, C₁₋₄alkanoylamino, C₁₋₄alkylS(O)ₐ wherein a is 0 or 2, C₁₋₄alkoxycarbonyl amino, C₁₋₄alkoxycarbonyl-N—(C₁₋₄alkyl)amino, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶;
R⁶ is selected from halo, nitro, C₁₋₄alkyl, C₂₋₄alkenyl, C₁₋₄alkoxy, C₁₋₄alkoxycarbonylamino, carbocyclyl and carbocyclylC₀₋₄alkylene-Z-; wherein R⁶ may be optionally substituted on carbon by one or more R⁸;
R⁵ is selected from C₁₋₄alkyl and C₁₋₄alkoxycarbonyl;
R⁸ is selected from halo; and
Z is —S(O)ₐ—, —O—, —C(O)— or —OC(O)NR¹⁰—; wherein a is 0 or 2; wherein R¹⁰ is selected from hydrogen.
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵; wherein
R² is a substituent on carbon and is selected from halo, nitro, cyano, amino, trifluoromethyl, trifluoromethoxy, C₁₋₄alkyl, C₁₋₄-alkoxy, C₁₋₄alkanoyl, N—(C₁₋₄alkyl)amino, N,N—(C₁₋₄alkyl)₂amino, C₁₋₄alkanoylamino, C₁₋₄alkylS(O)ₐ wherein a is 0 to 2, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶;
R⁶ is-selected from halo, nitro, cyano, trifluoromethyl, C₁₋₄alkyl, C₂₋₄alkenyl, C₁₋₄alkoxy, N,N—(C₁₋₄alkyl)₂amino, C₁₋₄alkylS(O)ₐ wherein a is 0 to 2, C₁₋₄alkoxycarbonylamino, carbocyclyl, heterocyclyl and carbocyclylC₀₋₄alkylene-Z-; wherein R⁶ may be optionally substituted on carbon by one or more R⁸;
R⁵ is selected from C₁₋₄alkyl, C₁₋₄alkanoyl and C₁₋₄alkoxycarbonyl;
Z is —S(O)ₐ—, —O—, —NR¹⁰—, —C(O)— or —OC(O)NR¹⁰—; wherein a is 0 to 2; wherein R¹⁰ is selected from hydrogen; and
R⁸ is selected from halo.

Y is hydrogen, methyl, ethyl, propyl, isopropyl, pentyl, butyl, t-butyl, allyl, ethynyl, phenyl, naphthyl, cyclopropyl, cyclopentyl, cyclohexyl, 1,2,3,4-tetrahydronaphthyl, indenyl, thienyl, furyl, thiazolyl, pyrazinyl, pyrrolyl, indolyl, quinolinyl, isoquinolinyl, pyrazolyl, isoxazolyl, benzofuranyl, 1,2,3-thiadiazolyl, 1,2,5-thiadiazolyl, pyrimidinyl, 2,1-benzisoxazolyl, 4,5,6,7-tetrahydro-2H-indazolyl, imidazo[2,1-b][1,3]thiazolyl, tetrahydrofuranyl, tetrahydropyranyl, piperidinyl, morpholinyl, 2,3-dihydro-1-benzofuryl, 2,3-dihydro-1,4-benzodioxinyl or pyridyl; wherein Y may be optionally substituted on carbon by one or more R²; wherein if said pyrrolyl, indolyl, piperidinyl, morpholinyl or pyrazolyl contains an —NH— moiety that nitrogen may be optionally substituted by a group selected from R⁵; wherein
R² is a substituent on carbon and is selected from fluoro, chloro, nitro, cyano, amino, trifluoromethyl, trifluoromethoxy, methyl, ethyl, t-butyl, methoxy, ethoxy, propoxy, isopropoxy, isobutoxy, t-butoxy, acetyl, methylamino, dimethylamino, acetamido, methylthio, mesyl, t-butoxycarbonylamino, N-(t-butoxycarbonyl)-N-(butyl)amino, phenyl, thienyl, isoxazolyl, morpholino, pyridyl, pyrazolyl, pyrrolidinyl, indolyl, 1,3-benzodioxolyl, isoindolinyl, pyrrolyl, phenoxy, phenylthio, benzyloxy, benzoyl, benzyloxycarbonylamino, thienylcarbonyl, pyrimidin-2-ylthio and morpholinosulphonyl; wherein R² may be optionally substituted on carbon by one or more groups selected from R⁶;
R⁶ is selected from fluoro, chloro, bromo, nitro, methyl, ethenyl, methoxy, t-butoxyoxycarbonylamino, phenyl, phenoxy and benzoyl; wherein R⁶ may be optionally substituted on carbon by one or more R⁸;
R⁵ is selected from methyl, ethyl and t-butoxycarbonyl; and
R⁸ is selected from bromo.
Y is hydrogen, methyl, ethyl, propyl, isopropyl, butyl, t-butyl, pentyl, naphthyl, phenyl, pyridyl, thienyl, furyl, cyclopropyl, cyclohexyl, thiazolyl, pyrazinyl, pyrrolyl, indolyl, quinolinyl, pyrazolyl, isoxazolyl, isoquinolinyl, indenyl, 1,2,3,4-tetrahydronaphthyl, benzofuranyl, 1,2,3-thiadiazolyl, 1,2,5-thiadiazolyl, pyrimidinyl, morpholinyl, piperidinyl, 2,1-benzisoxazolyl, 4,5,6,7-tetrahydro-2H-indazolyl, isoindolinyl, tetrahydrofuryl, imidazo[2,1-b][1,3]thiazolyl, cyclopentyl, 2,3-dihydro-1,4-benzodioxinyl, tetrahydropyranyl, 2,3-dihydrobenzofuranyl, 1,3-benzodioxolyl, benzothienyl, chromanyl, 1,2,3,4-tetrahydroquinolinyl, 1,3-benzothiazolyl, 3,4-dihydro-2H-benzodioxepinyl, (3r)-adamantanyl, pyrrolidinyl, oxazolyl, 4,5,6,7-tetrahydro-1H-indolyl, quinoxalinyl or 4,5,6,7-tetrahydro-1-benzofuryl; wherein Y may be optionally substituted on carbon by one or more R²; wherein if any heterocyclyl contains an —NH— moiety that nitrogen may be optionally substituted by a group selected from R⁵;
R² is fluoro, chloro, bromo, cyano, trifluoromethyl, nitro, amino, methyl, ethyl, isopropyl, t-butyl, methoxy, ethoxy, propoxy, isopropoxy, isobutoxy, t-butoxy, acetyl, phenyl, thienyl, morpholino, isoxazolyl, pyrazolyl, pyridyl, pyrrolidinyl, methylamino, isopropylamino, butylamino, dimethylamino, methylthio, mesyl, indolyl, morpholinosulphonyl, acetylamino, benzyloxy, 1,3-benzodioxolyl, thienylcarbonyl, phenoxy, phenylthio, pyrimidinylthio, t-butoxycarbonylamino, trifluoromethoxy, benzoyl, pyrrolyl, N-butyl-N-t-butoxycarbonylamino, N-methyl-N-t-butoxycarbonylamino, N-methylsulphamoyl, N,N-dimethylsulphamoyl, N-(t-butyl)sulphamoyl, piperidinyl, dimethylaminothiocarbonylthio, pyridazinyl or anilino; wherein R² may be optionally substituted on carbon by one or more groups selected from R⁶;
R⁶ is fluoro, chloro, bromo, cyano, nitro, trifluoromethyl, methyl, isopropyl, t-butyl, methoxy, ethoxy, t-butoxy, methylthio, phenyl, phenoxy, ethenyl, t-butoxycarbonylamino, dimethylamino or morpholino; wherein R⁶ may be optionally substituted on carbon by one or more R⁸
R⁵ is selected from methyl, ethyl, t-butoxycarbonyl and acetyl;
Z is —S(O)ₐ—, —O—, —NR¹⁰—, —C(O)— or —OC(O)NR¹⁰—; wherein a is 0 to 2; wherein R¹⁰ is selected from hydrogen; and
R⁸ is bromo.
X and Y together form 6-chloronaphth-2-ylmethyl, benzyl, thien-2-ylmethyl, carbamoyl, N,N-dimethylcarbamoyl, N,N-diisopropylcarbamoyl, N-(phenyl)carbamoyl, N-(2-fluorophenyl)carbamoyl, N-(4-fluorophenyl)carbamoyl, N-(3,4-difluorophenyl)carbamoyl, N-(3-chlorophenyl)carbamoyl, N-(3-methylphenyl)carbamoyl, N-(benzyl)carbamoyl, morpholinocarbonyl, piperidin-1-ylcarbonyl, pyrid-4-yl, 4-fluorophenyl, 4-trifluoromethylphenyl, 4-acetylphenyl, 4-acetamidophenyl, 4-methoxyphenyl, pyrimidin-2-yl, phenoxycarbonyl, methoxycarbonyl, ethoxycarbonyl, allyloxycarbonyl, 2-methoxyethoxycarbonyl, benzyloxycarbonyl, isopropoxycarbonyl, 4-fluorophenoxycarbonyl, 4-methoxyphenoxycarbonyl, pyrrol-2-ylcarbonyl, 4-bromopyrrol-2-ylcarbonyl, 1-methylpyrrol-2-ylcarbonyl, 4-nitropyrrol-2-ylcarbonyl, 1,5-dimethylpyrrol-2-ylcarbonyl, 2,5-dimethylpyrrol-3-ylcarbonyl, thien-2-ylcarbonyl, thien-3-ylcarbonyl, 3-chlorothien-2-ylcarbonyl, 3-methylthien-2-ylcarbonyl, 5-chlorothien-2-ylcarbonyl, 3-bromothien-2-ylcarbonyl, 5-bromothien-2-ylcarbonyl, 5-methylthien-2-ylcarbonyl, 2-chloro-3-methoxythien-4-ylcarbonyl, thien-2-ylmethylcarbonyl, 5-mesylthien-2-ylcarbonyl, fur-2-ylcarbonyl, 5-bromofur-2-ylcarbonyl, 3-methylfur-2-ylcarbonyl, fur-3-ylcarbonyl, 2,5-dimethylfur-3-ylcarbonyl, 2,3-dimethylfur-5-ylcarbonyl, 2-methylfur-3-ylcarbonyl, 2-methyl-5-t-butylfur-3-ylcarbonyl, 5-trifluoromethylfur-2-ylcarbonyl, pyrid-2-ylcarbonyl, cyclopropylcarbonyl, cyclopentylcarbonyl, cyclohexylcarbonyl, benzoyl, 3-methylbenzoyl, 4-methylbenzoyl, 2-ethylbenzoyl, 3-ethylbenzoyl, 4-ethylbenzoyl, 4-t-butylbenzoyl, 2-fluorobenzoyl, 3-fluorobenzoyl, 4-fluorobenzoyl, 2-chlorobenzoyl, 3-chlorobenzoyl, 4-chlorobenzoyl, 2-bromobenzoyl, 3-bromobenzoyl, 4-bromobenzoyl, 2-(t-butoxycarbonylamino)benzoyl, 4-(z-butoxycarbonylamino)benzoyl, 2,3-difluorobenzoyl, 2,4-difluorobenzoyl, 2,5-difluorobenzoyl, 3,4-difluorobenzoyl, 3,5-difluorobenzoyl, 2,3,4-trifluorobenzoyl, 3,4,5-trifluorobenzoyl, 2,4,5-trifluorobenzoyl, 2,3,4,5-tetrafluorobenzoyl, 2-cyanobenzoyl, 3-cyanobenzoyl, 4-cyanobenzoyl, 2-methoxybenzoyl, 3-methoxybenzoyl, 4-methoxybenzoyl, 2,3-dimethoxybenzoyl, 2,4dimethoxybenzoyl, 3,5-dimethoxybenzoyl, 2,3,4-trimethoxybenzoyl, 2,4,6-trimethoxybenzoyl, 2-ethoxybenzoyl, 3-ethoxybenzoyl, 4-ethoxybenzoyl, 3-propoxybenzoyl, 4-ispropoxybenzoyl, 3-(isobutoxy)benzoyl, 3-(t-butoxy)benzoyl, 4-(t-butoxy)benzoyl, 2-trifluoromethylbenzoyl, 3-trifluoromethylbenzoyl, 4-trifluoromethylbenzoyl, 4-methylaminobenzoyl, 4-dimethylaminobenzoyl, 2-methylthiobenzoyl, 4-methylthiobenzoyl, 2-nitrobenzoyl, 4-nitrobenzoyl, 3-(benzyloxycarbonylamino)benzoyl, 2-(phenethyl)benzoyl, 2-(phenoxymethyl)benzoyl, 4-(phenoxymethyl)benzoyl, 2-(trifluoromethoxy)benzoyl, 3-(trifluoromethoxy)benzoyl, 3-phenoxybenzoyl, 4-phenoxybenzoyl, 3-benzoylbenzoyl, 3-benzyloxybenzoyl, 3-(allyloxy)benzoyl, 4-pyrrol-1-ylbenzoyl, 4-(t-butoxycarbonylaminomethyl)benzoyl, 4-[N-(t-butoxycarbonyl)-N-(butyl)amino]benzoyl, 2-fluoro-5-methoxybenzoyl, 3-fluoro-4-methoxybenzoyl, 5-fluoro-2-methoxybenzoyl, 3-fluoro-4-methylbenzoyl, 2-methyl-3-fluorobenzoyl, 2chloro-3-methoxybenzoyl, 2-methoxy-3-methylbenzoyl, 3-methoxy-4-methylbenzoyl, 2-methoxy-4-methylbenzoyl, 2-methyl-3-methoxybenzoyl, 2-methyl-4-methoxybenzoyl, 3-methyl-4-methoxybenzoyl, 2,4-dimethoxy-3-methylbenzoyl, 3-(morpholinosulphonyl)benzoyl, 4-(morpholinosulphonyl)benzoyl, 3-benzyloxy-4-methoxybenzoyl, 2-ethylbutyryl, 4-(2,4-dimethylphenyl)butyryl, 4-(indol-3-yl)butyryl, 4-(5-bromothien-2-ylcarbonyl)butyryl, 4-morpholinobenzoyl, isoxazole-5-ylcarbonyl, 3-methylisoxazole-5-ylcarbonyl, 3,5-dimethylisoxazol-4-ylcarbonyl, 4-(pyrazol-1-yl)benzoyl, thiazol-4-ylcarbonyl, 2-methylthiazol-4-ylcarbonyl, 3-chlorothiazol-5-ylcarbonyl, 2,4-dimethylthiazol-5-ylcarbonyl, 2-(pyrid-2-yl)-4-methylthiazol-5-ylcarbonyl, 2-(pyrrolidin-1-yl)pyrazin-6-ylcarbonyl, 2-phenylbenzoyl, 4-phenylbenzoyl, 2-(2-nitrophenyl)benzoyl, 3-(4-fluorophenyl)benzoyl, 4-acetylbenzoyl, indol-6-ylcarbonyl, indol-7-ylcarbonyl, 5-fluoroindol-2-ylcarbonyl, 1-methylindol-3-ylcarbonyl, 3-methylindol-1-ylcarbonyl, 5-methoxyindol-2-ylcarbonyl, isoquinoline-1-ylcarbonyl, quinoline-2-ylcarbonyl, quinoline-3-ylcarbonyl, quinoline-4-ylcarbonyl, quinoline-6-ylcarbonyl, 2-methylquinoline-6-ylcarbonyl, 3-methylinden-2-ylcarbonyl, 1,2,3,4-tetrahydronaphth-5-ylcarbonyl, benzofuran-2-ylcarbonyl, 1,2,3-thiadiazol-4-ylcarbonyl, 1,2,5-thiadiazol-3-ylcarbonyl, pyrazol-3-ylcarbonyl, 1-methylpyrazol-3-ylcarbonyl, 5-methylpyrazol-3-ylcarbonyl, 1,5-dimethylpyrazol-3-ylcarbonyl, 1-ethyl-3-methylpyrazol-5-ylcarbonyl, 1-methyl-5-chloropyrazol-4-ylcarbonyl, 1-methyl-3-t-butylpyrazol-5-ylcarbonyl, 2,1-benzisoxazol-3-ylcarbonyl, 2-(2-chlorophenyl)ethynylcarbonyl, 3-(5-bromo-1,3-benzodioxol-6-yl)propionyl, 2-methylpropionyl, 2,2-dimethylpropionyl, 2-ethylheptanoyl, 4,5,6,7-tetrahydro-2H-indazol-3-ylcarbonyl, 6-methylimidazo[2,1-b][1,3]thiazol-5-ylcarbonyl, N-(t-butoxycarbonyl)piperidin-3-ylcarbonyl, N-(t-butoxycarbonyl)piperidin-4-ylcarbonyl, N-(t-butoxycarbonyl)morpholin2-ylcarbonyl, tetrahydrofuran-2-ylcarbonyl, tetrahydrofuran-3-ylcarbonyl, 2,3-dihydro-1,4-benzodioxin-2-ylcarbonyl, tetrahydropyranylcarbonyl, 2,3-dihydro-1-benzofur-2-ylcarbonyl, acetyl, (3,5-dimethylisoxazol-4-yl)acetyl, (4-fluorophenyl)acetyl, (2-nitrophenyl)acetyl, (4-bromobenzoylmethylthio)acetyl, (2,4-dichloro-6-methoxyphenoxy)acetyl, (2-nitro-4-chlorophenylthio)acetyl, (pyrimidin-2-ylthio)acetyl, (isoindolin-2-yl)acetyl, thien-2-ylsulphonyl, mesyl, ethylsulphonyl, isopropylsulphonyl, butylsulphonyl, 2-methylphenylsulphonyl, 3-methylphenylsulphonyl, 4-methylphenylsulphonyl, 2,5-dimethylphenylsulphonyl, 4-ethylphenylsulphonyl, 3-methoxyphenylsulphonyl, 4-methoxyphenylsulphonyl, 2-fluorophenylsulphonyl, 3-fluorophenylsulphonyl, 4-fluorophenylsulphonyl, 2-chlorophenylsulphonyl, 3-chlorophenylsulphonyl, 4-chlorophenylsulphonyl, 2-bromophenylsulphonyl, 3-bromophenylsulphonyl, 4-bromophenylsulphonyl, 2-trifluoromethylsulphonyl, 3-trifluoromethylsulphonyl, 4-trifluoromethylsulphonyl, 4-acetamidophenylsulphonyl, 2,4-difluorophenylsulphonyl, 2,6-difluorophenylsulphonyl, 2,4,5-trifluorophenylsulphonyl, 2-cyanophenylsulphonyl, 2-chloro-4-fluorophenylsulphonyl, 2-chloro-6-methylphenylsulphonyl, 3-fluoro-6-methylphenylsulphonyl, 2-methoxy-5-methylphenylsulphonyl, 2-nitro-4-methoxyphenylsulphonyl, 3-chloro-4-aminophenylsulphonyl, 2-chloro-4-cyanophenylsulphonyl, benzylsulphonyl, 4-fluorobenzylsulphonyl, thien-3-ylsulphonyl, 5-chlorothien-2-ylsulphonyl, 2,5-dichlorothien-3-ylsulphonyl, 1,3-dimethyl-5-chloropyrazol-4-ylsulphonyl, 3,5-dimethylisoxazol-4-ylsulphonyl and (4-fluoroanilino)thiocarbonyl.
X and Y together form hydrogen, t-butoxycarbonyl, carbamoyl, N,N-dimethylcarbamoyl, N,N-diisopropylcarbamoyl, acetyl, mesyl, isopropylsulphonyl, ethylsulphonyl, butylsulphonyl, methoxycarbonyl, ethoxycarbonyl, allyloxycarbonyl, 2-methoxyethoxycarbonyl, isopropylcarbonyl, hept-3-ylcarbonyl, t-butylcarbonyl, pent-3-ylcarbonyl, isopropoxycarbonyl, dimethylaminothiocarbonylthioacetyl, 3,3,3-trifluoropropionyl, 4,4,4-trifluorobutyryl, 2-methyl-4,4,4-trifluorobutyryl, 2-(t-butoxycarbonylamino)acetyl, 2-(N-methyl-t-butoxycarbonylamino)acetyl, 2-aminoacetyl, pyrid-4-yl, 4-fluorophenyl, pyrimidin-2-yl, 4-trifluoromethylphenyl, 4-acetylphenyl, 4-acetylaminophenyl, 4-methoxyphenyl, 6-chloronaphth-2-ylmethyl, benzyl, thien-2-ylmethyl, 4-acetylbenzoyl, 3-allyloxybenzoyl, 2-aminobenzoyl, 3-benzoylbenzoyl, 3-benzyloxybenzoyl, 4-benzyloxybenzoyl, 3-(benzyloxycarbonylamino)benzoyl, 2-bromobenzoyl, 3-bromobenzoyl, 4-bromobenzoyl, benzoyl, 4-(N-butyl-t-butoxycarbonylamino)benzoyl, 2-t-butoxycarbonylaminobenzoyl, 4-t-butoxycarbonylaminobenzoyl, 4-(t-butoxycarbonylaminomethyl)benzoyl, 3-t-butoxybenzoyl, 4-t-butoxybenzoyl, 4-butylaminobenzoyl, 4-t-butylbenzoyl, 4-difluoromethoxybenzoyl, 2-chlorobenzoyl, 3-chlorobenzoyl, 4-chlorobenzoyl, 2-cyanobenzoyl, 3-cyanobenzoyl, 4-cyanobenzoyl, 2-difluoromethoxybenzoyl, 4-difluoromethoxybenzoyl, 4-dimethylaminobenzoyl, 4-(3-dimethylaminopyridazin-6-yl)benzoyl, benzoyl, 2-ethoxybenzoyl, 3-ethoxybenzoyl, 4-ethoxybenzoyl, 4-(2-ethoxyethoxy)benzoyl, 2-ethylbenzoyl, 3-ethylbenzoyl, 4-ethylbenzoyl, 2-fluorobenzoyl, 3-fluorobenzoyl, 4-fluorobenzoyl, 3-(4fluorophenyl)benzoyl, 3-isobutoxybenzoyl, 4-isopropoxybenzoyl, 4-isopropylaminobenzoyl, 2-isopropylbenzoyl, 2-methoxybenzoyl, 3-methoxybenzoyl, 4-methoxybenzoyl, 2-methylbenzoyl, 4-methylaminobenzoyl, 4-methylbenzoyl, 2-methylthiobenzoyl, 4-methylthiobenzoyl, 4-morpholinobenzoyl, 3-morpholinosulphonylbenzoyl, 4-morpholinosulphonylbenzoyl, 2-nitrobenzoyl, 4-nitrobenzoyl, 2-(2-nitrophenyl)benzoyl, 2-phenethylbenzoyl, 3-phenoxybenzoyl, 4-phenoxybenzoyl, 2-phenoxymethylbenzoyl, 2-phenylbenzoyl, 4-phenylbenzoyl, 4-piperidin-1-ylbenzoyl, 3-propoxybenzoyl, 4-pyrazol-1-ylbenzoyl, 4-pyrrol-1-ylbenzoyl, 2-trifluoromethoxybenzoyl, 3-trifluoromethoxybenzoyl, 4-trifluoromethoxybenzoyl, 2-trifluoromethylbenzoyl, 3-trifluoromethylbenzoyl, 4-trifluoromethylbenzoyl, 2,3-difluorobenzoyl, 2,4-difluorobenzoyl, 2,5-difluorobenzoyl, 3,4-difluorobenzoyl, 3,5-difluorobenzoyl, 2,4-dichlorobenzoyl, 3,4-dichlorobenzoyl, 2,3-dimethoxybenzoyl, 2,4-dimethoxybenzoyl, 3,5-dimethoxybenzoyl, 3,5-ditrifluoromethylbenzoyl, 2-(3-trifluoromethylanilino)benzoyl, 2-fluoro-6-methoxybenzoyl, 2-fluoro-4-chlorobenzoyl, 2-fluoro-4-cyanobenzoyl, 2-fluoro-5-methoxybenzoyl, 2-fluoro-5-trifluoromethylbenzoyl, 2-fluoro-5-methylbenzoyl, 3-fluoro-4-methoxybenzoyl, 3-fluoro-4-methylbenzoyl, 3-fluorotrifluoromethylbenzoyl, 2-methyl-3-fluorobenzoyl, 2-methyl-4-metboxybenzoyl, 2-methyl-3-methoxybenzoyl, 3-methyl-4-methoxybenzoyl, 2-methoxy-3-fluorobenzoyl, 2-methoxy-5-fluorobenzoyl, 2-methoxy-4-methylbenzoyl, 2-methoxy-3-methylbenzoyl, 2-methoxy-4-chlorobenzoyl, 3-methoxy-4-methylbenzoyl, 3-methoxy-4-chlorobenzoyl, 3-benzyloxy-4-methoxybenzoyl, 2-(t-butylsulphamoyl)-5-chlorobenzoyl, 2-trifluoromethyl-4-fluorobenzoyl, 3-trifluoromethyl-4-fluorobenzoyl, 3-trifluoromethyl-4-methoxybenzoyl, 3-trifluoromethyl-4-methylbenzoyl, 3-trifluoromethyl-4-chlorobenzoyl, 2-chloro-4-fluorobenzoyl, 2-chloro-5-fluorobenzoyl, 2-chloro-3-methoxybenzoyl, 2-chloro-5-trifluoromethylbenzoyl, 2-chloro-5-(pyrrol-1-yl)benzoyl, 2-chloro-4-morpholinobenzoyl, 3-chloro-4-fluorobenzoyl, 3-chloro-4-trifluorormethoxybenzoyl, 3-mesyl-4-chlorobenzoyl, 2,3,4-trifluorobenzoyl, 2,4,5-trifluorobenzoyl, 3,4,5-trifluorobenzoyl, 2,3,4-trimethoxybenzoyl, 2,4,6-trimethoxybenzoyl, 2,4-dimethoxy-3-methylbenzoyl, 2-chloro-4,5-dimethoxybenzoyl, 2,3,4,5-tetrafluorobenzoyl, cyclopropylcarbonyl, 1-phenylcyclopropylcarbonyl, 1-(4-methoxyphenyl)cyclopropylcarbonyl, cyclopentylcarbonyl, 1-phenylcyclopentlycarbonyl, cyclohexylcarbonyl, 4-(4-chlorophenoxy)cyclohexylcarbonyl, 4,4-difluorocyclohexylcarbonyl, 3-methylinden-2-ylcarbonyl, 1,2,3,4-tetrahydronaphth-5-ylcarbonyl, (3r)-adamantan-1-ylcarbonyl, thien-2-ylcarbonyl, thien-3-ylcarbonyl, 2-chloro-3-methoxylthien-4-ylcarbonyl, 3-methylthien-2-ylcarbonyl, 5-methylthien-2-ylcarbonyl, 3-chlorothien-2-ylcarbonyl, 5-chlorothien-2-ylcarbonyl, 5-bromothien-2-ylcarbonyl, 3-bromothien-2-ylcarbonyl, 5-methylthien-2-ylcarbonyl, 5-(pyrid-2-yl)thien-2-ylcarbonyl, 5-acetylthien-2-ylcarbonyl, 5-methylthiothien-2-ylcarbonyl, fur-2-ylcarbonyl, fur-3-ylcarbonyl, 5-bromofur-2-ylcarbonyl, 5-trifluoromethylfur-2-ylcarbonyl, 3-methylfur-2-ylcarbonyl, 5-ethoxyfur-2-ylcarbonyl, 2-methyl-5-t-butylfur-3-ylcarbonyl, 2,5-dimethylfur-3-ylcarbonyl, 2,3-dimethylfur-5-ylcarbonyl, 2-methylfur-3-ylcarbonyl, 5-methylfur-2-ylcarbonyl, 5-(4-chlorophenyl)fur-2-ylcarbonyl, 5-(dimethylaminomethyl)fur-2-ylcarbonyl, 5-(morpholinomethyl)fur-2-ylcarbonyl, 5-phenylfur-2-ylcarbonyl, 2-trifluoromethyl-5-phenylfur-3-ylcarbonyl, 2-methyl-5-(N,N-dimethylsulphamoyl)fur-3-ylcarbonyl, thiazol-4-ylcarbonyl, 2-methylthiazol-4-ylcarbonyl, 2-phenylthiazol-4-ylcarbonyl, 2-(4-chlorophenyl)thiazol-4-ylcarbonyl, thiazol-5-ylcarbonyl, 2-phenyl-4-methylthiazol-5-ylcarbonyl, 2-chlorothiazol-5-ylcarbonyl, 2,4dimethylthiazol-5-ylcarbonyl, 2-(pyrid-2-yl)-4-methylthiazol-5-ylcarbonyl, 2-(4-trifluoromethylphenyl)-4-methylthiazol-5-ylcarbonyl, pyrazin-2-ylcarbonyl, 2-methylaminopyrazin-6-ylcarbonyl, 2-(pyrrolidin-1-yl)pyrazin-6-ylcarbonyl, pyrrol-2-ylcarbonyl, 1-methylpyrrol-2-ylcarbonyl, 4-bromopyrrol-2-ylcarbonyl, 1,2-dimethylpyrrol-5-ylcarbonyl, 1,5-dimethylpyrrol-3-ylcarbonyl, 4-nitropyrrol-2-ylcarbonyl, indol-2-ylcarbonyl, 1-acetylindol-2-ylcarbonyl, 5-fluoroindol-2-ylcarbonyl, 5-trifluoromethoxyindol-2-ylcarbonyl, 5,7-difluoroindol-2-ylcarbonyl, indol-5-ylcarbonyl, indol-6-ylcarbonyl, indol-7-ylcarbonyl, 1-methylindol-3-ylcarbonyl, 1-methylindol-7-ylcarbonyl, quinoline-2-ylcarbonyl, quinoline-3-ylcarbonyl, quinoline-4-ylcarbonyl, quinoline-6-ylcarbonyl, 2-methylquinolin-6-ylcarbonyl, pyrid-2-ylcarbonyl, 3-methylpyrid-2-ylcarbonyl, 6-methylpyrid-2-ylcarbonyl, 3-propoxypyrid-2-ylcarbonyl, 3-(4-chlorobenzoyl)pyrid-2-ylcarbonyl, 3-chloro-5-trifluoromethylpyrid-2-ylcarbonyl, pyrid-3-ylcarbonyl, 6-trifluoromethylpyrid-3-ylcarbonyl, 4-trifluoromethylpyrid-3-ylcarbonyl, 2-(3-trifluoromethylanilino)pyrid-3-ylcarbonyl, isoquinolin-1-ylcarbonyl, benzofuran-2-ylcarbonyl, 2-methylbenzofuran-6-ylcarbonyl, isoxazol-5-ylcarbonyl, 3-methylisoxazol-5-ylcarbonyl, 3,5-dimethylisoxazol-4-ylcarbonyl, 1,2,3-thiadiazol-4-ylcarbonyl, 1,2,5-thiadiazol-3-ylcarbonyl, pyrazol-3-ylcarbonyl, 1-methylpyrazol-3-ylcarbonyl, 5-methylpyrazol-3-ylcarbonyl, 1,5-dimethylpyrazol-3-ylcarbonyl, 1-ethyl-3-methylpyrazol-5-ylcarbonyl, 1-methyl-5-chloropyrazol-3-ylcarbonyl, 1-methyl-3-t-butylpyrazol-5-ylcarbonyl, morpholinocarbonyl, piperidin-1-ylcarbonyl, 4-(4-fluorobenzoyl)piperidin-1-ylcarbonyl, 1-(t-butoxycarbonyl)-4-phenylpiperidin-4-ylcarbonyl, 2,1-benzisoxazol-3-ylcarbonyl, 4,5,6,7-tetrahydro-2H-indazol-3-ylcarbonyl, 6-methylimidazo[2,1-b][1,3]thiazol-5-ylcarbonyl, 1-(t-butoxycarbonyl)-piperdin-3-ylcarbonyl, 1-(t-butoxycarbonyl)-piperdin-4-ylcarbonyl, tetrahydrofur-2-ylcarbonyl, tetrahydrofur-2-ylcarbonyl, tetrahydrofur-3-ylcarbonyl, 2,3-dihydro-1,4-benzodioxin-2-ylcarbonyl, 4-(t-butoxycarbonyl)-morpholin-2-ylcarbonyl, tetrahydropyran-4-ylcarbonyl, 2,3-dihydrobenzofuran-2-ylcarbonyl, 2,3-dihydrobenzofuran-5-ylcarbonyl, 2,3-dihydrobenzofuran-7-ylcarbonyl, 1,3-benzodioxol-4-ylcarbonyl, 1,3-benzodioxol-5-ylcarbonyl, 2,2-difluoro-1,3-benzodioxol-4-ylcarbonyl, 2,2-difluoro-1,3-benzodioxol-5-ylcarbonyl, benzothien-2-ylcarbonyl, chroman-2-ylcarbonyl, 2,2-dimethylchroman-6-ylcarbonyl, 1,2,3,4-tetrahydroquinolin-6-ylcarbonyl, 1,3-benzothiazol-6-ylcarbonyl, 3,4-dihydro-2H-benzodioxepin-7-ylcarbonyl, pyrrolidin-1-ylcarbonyl, 2-phenyl-5-trifluoromethyloxazol-4-ylcarbonyl, 2-methyl-5-trifluoromethyloxazol-4-ylcarbonyl, 4,5,6,7-tetrahydro-1 1-indol-2-ylcarbonyl, quinoxaline-2-ylcarbonyl, 2-methyl-4,5,6,7-tetrahydro-1-benzofur-3-ylcarbonyl, 2-(thien-2-yl)acetyl, 2-(3,5-dimethylisoxazol-4-yl)acetyl, 2-(4-fluorophenyl)acetyl, 2-(4-trifluoromethylphenyl)acetyl, 2-(2-nitrophenyl)acetyl, 2-(4-bromobenzoylmethylthio)acetyl, 2-(2,4-dichloro-6-methoxyphenoxy)acetyl, 2-(pyrimidin-2-ylthio)acetyl, 2-(isoindolin-2-yl)acetyl, 2-(phenoxy)acetyl, 2-(4-fluorophenoxy)acetyl, 2-(4-isopropylphenoxy)acetyl, 2-(3-chlorophenoxy)acetyl, 2-(3-methoxyphenoxy)acetyl, 2-(4-t-butylphenoxy)acetyl, 2-(t-butoxyphenoxy)acetyl, 2-(4-cyanophenoxy)acetyl, 2-(3-trifluoromethylphenoxy)acetyl, 2-(4-methylthiophenoxy)acetyl, 2-(3,5-dichlorophenoxy)acetyl, 2-(2-trifluoromethylphenyl)acetyl, 2-(3-trifluoromethyl-4-fluorophenyl)acetyl, 2-(3-trifluoromethyl-5-fluorophenyl)acetyl, 2-(3,5-ditrifluoromethylphenyl)acetyl, 4-(2,4-dimethylphenyl)butyryl, 4-indol-3-ylbutyryl, 4-(5-bromothien-2-ylcarbonyl)butyryl, 2-(4-chlorophenoxy)-2-(methyl)butyryl, 3-(2-chlorophenyl)propioloyl, 3-(5-bromo-1,3-benzodioxol-6-yl)propionyl, 3-(3-methylindol-1-yl)propionyl, 3-(4-trifluoromethylphenyl)propionyl, 2-(4-chlorophenoxy)propionyl, 2-(4-chlorophenyl)-2-(methyl)propionyl, 2-(4-chlorophenoxy)-2-(methyl)propionyl, 2-(phenoxy)-2-(methyl)propionyl, 2-(3-trifluoromethylphenoxy)-2-(methyl)propionyl, 4-acetylaminophenylsulphonyl, 2-bromophenylsulphonyl, 3-bromophenylsulphonyl, 4-bromophenylsulphonyl, 4-chlorophenylsulphonyl, 2-cyanophenylsulphonyl, 4-ethylphenylsulphonyl, 2-fluorophenylsulphonyl, 3-fluorophenylsulphonyl, 4-fluorophenylsulphonyl, 2-chlorophenylsulphonyl, 3-chlorophenylsulphonyl, 3-methoxyphenylsulphonyl, 4-methoxyphenylsulphonyl, 2-methylphenylsulphonyl, 3-methylphenylsulphonyl, 4-methylphenylsulphonyl, 2-trifluoromethylphenylsulphonyl, 3-trifluoromethylphenylsulphonyl, 4-trifluoromethylphenylsulphonyl, 2,5-dimethylphenylsulphonyl, 2,4-difluorophenylsulphonyl, 2,6-difluorophenylsulphonyl, 2-chloro-4-fluorophenylsulphonyl, 2-methyl-5-fluorophenylsulphonyl, 2-methoxy-5-methylphenylsulphonyl, 2-methyl-6-chlorophenylsulphonyl, 2-nitro-4-methoxyphenylsulphonyl, 3chloro-4-aminophenylsulphonyl, 2-chloro-4-cyanophenylsulphonyl, 2,4,5-trifluorophenylsulphonyl, thien-2-ylsulphonyl, thien-3-ylsulphonyl, 5-chlorothien-2-ylsulphonyl, 2,5-dichlorothien-3-ylsulphonyl, 1,3-dimethyl-5-chloropyrazol-4-ylsulphonyl, 3,5-dimethylisoxazol-4-ylsulphonyl, benzylsulphonyl, 4-fluorobenzylsulphonyl, anilinocarbonyl, N-methylanilinocarbonyl, 2-fluoroanilinocarbonyl, 4-fluoroanilinocarbonyl, 4-fluoroanilinothiocarbonyl, 3-chloroanilinocarbonyl, 3-methylanilinocarbonyl, 2-ethylanilinocarbonyl, 2-trifluoromethylanilinocarbonyl, 2,3-difluoroanilinocarbonyl, 2,5-difluoroanilinocarbonyl, 2,6-difluoroanilinocarbonyl, 3,4-difluoroanilinocarbonyl, 2,6-dimethylaniliocarbonyl, 4-(pyrid-2-yl)anilinocarbonyl, N-methyl-4-fluoroanilinocarbonyl, benzylaminocarbonyl, 4-methoxybenzylaminocarbonyl, 4-methylbenzylaminocarbonyl, 2-fluorobenzylaminocarbonyl, 3-fluorobenzylaminocarbonyl, phenoxycarbonyl, benzyloxycarbonyl, 4-fluorophenoxycarbonyl, 4-methoxyphenoxycarbonyl, [(1R)-1-phenylethyl]aminocarbonyl or iminophenylmethyl.
R¹² is 4-methyl.
R¹² is 4-ethyl.
R¹²is 4-propyl.
R¹² is 3-methyl.
m is 0.
m is 1.
q is 0.
q is 1.

According to a further feature of the invention there is provided the use of a compound of formula (I) wherein:
Ring A is phenyl;
R¹ is selected from halo or C₁₋₄alkyl;
n is 1;
X is —C(O)—, —S(O)₂— or —CH₂—;
Y is phenyl, thienyl, methyl, furyl, cyclopropyl or cyclohexyl; wherein Y may be optionally substituted on carbon by one or more R⁷; and
R² is a substituent on carbon and is selected from halo or C₁₋₄alkyl;
   or a pharmaceutically acceptable salt thereof;
q is 0;
   in the manufacture of a medicament for use in the inhibition of 11βHSD1.

According to a further feature of the-invention there is provided the use of a compound of formula (I) wherein:
Ring A is selected from phenyl, 1,3-benzodioxolyl, thienyl, cyclopentyl, pyridyl or furyl;
R¹ is a substituent on carbon and is selected from halo, C₁₋₄alkyl, C₁₋₄alkoxy, carbocyclyl and carbocyclylC₀₋₄alkylene-Z-; wherein R¹ may be optionally substituted on carbon by one or more groups selected from R³; wherein R³ is halo; and Z is —S(O)ₐ—; wherein a is 2;
n is 0-2;-wherein the values of R¹ may be the same or different;
X is a direct bond, —C(O)—, —S(O)₂—, —C(O)NR¹¹—, —C(S)NR¹¹—, —C(O)O— or —CH₂—; wherein R¹¹ is selected from hydrogen and methyl;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵; wherein
R² is a substituent on carbon and is selected from halo, nitro, cyano, amino, trifluoromethyl, trifluoromethoxy, C₁₋₄alkyl, C₁₋₄alkoxy, C₁₋₄alkanoyl, N—(C₁₋₄alkyl)amino, N,N—(C₁₋₄alkyl)₂amino; C₁₋₄alkanoylamino, C₁₋₄alkylS(O)ₐ wherein a is 0 or 2, o, carbocyclyl and carbocyclylC₀₋₄alkylene-Z-; wherein R⁶ may be optionally substituted on carbon by one or more R⁸;
R⁵ is selected from C₁₋₄alkyl and C₁₋₄alkoxycarbonyl;
R⁸ is selected from halo; and
Z is —S(O)ₐ—, —O—, —C(O)— or —OC(O)NR¹⁰—; wherein a is 0 or 2; wherein R¹⁰ is selected from hydrogen;
R¹² is methyl or ethyl;
m is 0 or 1; and
q is 0 or 1;
   or a pharmaceutically acceptable salt thereof;
   in the manufacture of a medicament for use in the inhibition of 11βHSD1.

According to a further feature of the invention there is provided the use of a compound of formula (I) wherein:
Ring A is phenyl, 1,3-benzodioxolyl, thienyl, cyclopentyl, pyridyl, furyl, thiazolyl, 1,3-benzothiazolyl, benzofuryl or benzothienyl;
R¹ is a substituent on carbon and is selected from halo, cyano, C₁₋₄alkyl, C₁₋₄alkoxy, N,N—(C₁₋₄alkyl)₂amino, C₁₋₄alkylS(O)ₐ wherein a is 0 to 2, carbocyclyl and carbocyclylC₀₋₄alkylene-Z-; wherein R¹ may be optionally substituted on carbon by one or more groups selected from R³; wherein
R³ is selected from halo, hydroxy, C₁₋₄alkoxy, heterocyclyl and carbocyclylC₀₋₄alkylene-Z-; and
Z is —S(O)ₐ— or —O—; wherein a is 0 to 2;
X is a direct bond, —C(O)—, —S(O)₂—, —C(O)NR¹¹—, —C(S)NR¹¹—, —C(O)O—, —C(═NR¹¹) or —CH₂—; wherein R¹¹ is selected from hydrogen, C₁₋₄alkyl, carbocyclyl and heterocyclyl;
Y is hydrogen, C₁₋₆alkyl, C₂₋₆-alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by a group selected from R⁵; wherein
R² is a substituent on carbon and is selected from halo, nitro, cyano, amino, trifluoromethyl, trifluoromethoxy, C₁₋₄alkyl, C₁₋₄alkoxy, C₁₋₄alkanoyl, N—(C₁₋₄alkyl)amino, N,N—(C₁₋₄alkyl)₂amino, C₁₋₄alkanoylamino, C₁₋₄alkylS(O)ₐ wherein a is 0 to 2, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more groups selected from R⁶;
R⁶ is selected from halo, nitro, cyano, trifluoromethyl, C₁₋₄alkyl, C₂₋₄alkenyl, C₁₋₄alkoxy, N,N—(C₁₋₄alkyl)₂amino, C₁₋₄alkylS(O)ₐ wherein a is 0 to 2, C₁₋₄alkoxycarbonylamino, carbocyclyl, heterocyclyl and carbocyclylC₀₋₄alkylene-Z-; wherein R⁶ may be optionally substituted on carbon by one or more R⁸;
R⁵ is selected from C₁₋₄alkyl, C₁₋₄alkanoyl and C₁₋₄alkoxycarbonyl;
Z is —S(O)ₐ—, —O—, —NR¹⁰—, —C(O)— or —OC(O)NR¹⁰—; wherein a is 0 to 2; wherein R¹⁰ is selected from hydrogen; and
R⁸ is selected from halo;
R¹² is hydroxy, methyl, ethyl or propyl;
m is 0 or 1; and
q is 0 or 1;
   or a pharmaceutically acceptable salt thereof;
   in the manufacture of a medicament for use in the inhibition of 11βHSD1.

In another aspect of the invention, suitable compounds of the invention are any one of the Examples or a pharmaceutically acceptable salt thereof.

In another aspect of the invention, suitable compounds of the invention are any one of the Reference Examples or a pharmaceutically acceptable salt thereof.

In another aspect of the invention, preferred compounds of the invention are Examples 57, 76, 101, 103, 161, 206, 210, 213, 215, 233 and 398 or a pharmaceutically acceptable salt thereof.

In a further aspect of the invention there is provided a compound selected from Group A:
1-[2-hydroxy-2-(2,3-dihydro-1,4-benzodioxin-2-yl)ethyl]-4-(4-fluorobenzoyl)piperidine;
1-(7-methyl-2,3-dihydro-1,4-benzodioxin-2-ylmethyl)-4-(benzoyl)piperidine;
1-(6-fluoro-2,3-dihydro-1,4-benzodioxin-2-ylmethyl)-4-(benzoyl)piperidine;
1-(7-fluoro-2,3-dihydro-1,4-benzodioxin-2-ylmethyl)-4-(benzoyl)piperidine;
1-[2-(6-methoxynaphth-2-yl)propionyl]-4-(4-fluorobenzoyl)piperidine;
1-(4-bromoindol-2-ylcarbonyl)-4-(benzoyl)piperidine; and
1-(3-phenyl-5-methylisoxazol-4-ylcarbonyl)-4-(4-fluorobenzoyl)piperidine;
   or a pharmaceutically acceptable salt thereof.

In a further aspect of the invention there is provided the use of a compound selected from Group B:
1-[2-((1H,3H)-2,4-dioxoquinazolin-3-yl)ethyl]-4-(4-fluorobenzoyl)piperidine;
1-[3-(napath-1-yloxy)propyl]-4-(4-fluorobenzoyl)piperidine;
1-[2-(2-methyl-4-oxo-4H-pyrido[1,2-a]pyrimidin-3-yl)ethyl]-4-4(4-fluorobenzoyl)piperidine;
4-(4-fluorobenzoyl)piperidine;
1-(t-butoxycarbonyl)-4-(benzoyl)piperidine;
1-(acetyl)-4-(4-fluorobenzoyl)piperidine;
1-(t-butoxycarbonyl)-4-(4-fluorobenzoyl)piperidine;
1-(2,4-trifluoromethyl-6-methoxybenzoyl)-4-(4-chlorobenzoyl)piperidine;
1-(3,4-dichlorophenylsulphonyl)-4-(4-methylbenzoyl)piperidine;
1-(2-nitro-4-trifluoromethylphenyl)-4-(benzoyl)piperidine;
1-(anilinocarbonyl)-4-(benzoyl)piperidine;
1-[3-(2,6-dichlorophenyl)-5-methylisoxazolylcarbonyl]-4-(benzoyl)piperidine;
1-(4-chlorobenzoyl)-4-(benzoyl)piperidine;
1-[(5-trifluoromethylpyrid-2-ylthio)acetyl]-4-(benzoyl)piperidine;
1-[(4-chlorophenylthio)acetyl]-4-(benzoyl)piperidine;
1-(fur-2-ylcarbonyl)-4-(benzoyl)piperidine;
1-(4-methyl-1,2,3-thiadiazol-5-ylcarbonyl)-4-(benzoyl)piperidine;
1-(thien-2-ylcarbonyl)-4-(benzoyl)piperidine;
1-(3-trifluoromethylbenzoyl)-4-(benzoyl)piperidine;
1-(propylaminothiocarbonyl)-4-(4-methylbenzoyl)piperidine;
1-(5-nitrofur-2-ylcarbonyl)-4-(2,3,4,5,6-pentamethylbenzoyl)piperidine;
1-(3,5-ditrifluoromethylphenylsulphonyl)-4-(4-methylbenzoyl)piperidine;
1-(3,5-dimethylisoxazol-4-ylsulphonyl)-4-(4-methylbenzoyl)piperidine;
1-(2,6-difluorobenzoyl)-4-(benzoyl)piperidine;
1,4-bis-(4-methylbenzoyl)piperidine;
1-(3,5-ditrifluoromethylphenylsulphonyl)-4-(2,4-difluorobenzoyl)piperidine;
1-(2,4-difluorophenylsulphonyl)-4-(2,4-difluorobenzoyl)piperidine;
1-(4-methylbenzoyl)-4-(2,4,6-trimethylbenzoyl)piperidine;
1-(4-chlorophenylsulphonyl)-4-(benzoyl)piperidine;
1-[2-((1H,3H)-2-thiocarbonyl-4-oxoquinazolin-3-yl)ethyl]-4-(4-fluorobenzoyl)piperidine;
1-(trifluoroacetyl)-4-(benzoyl)piperidine;
1-(3,5-dimethylisoxazol-4-ylsulphonyl)-4-(benzoyl)piperidine;
1-(4-t-butylbenzoyl)-4-(benzoyl)piperidine;
1-(2,4-dimethylthiazol-5-ylsulphonyl)-4-(benzoyl)piperidine;
1-[(4-chlorophenylsulphonyl)acetyl]-4-(benzoyl)piperidine;
1-(4-chloroanilinocarbonyl)-4-(benzoyl)piperidine;
1-[3-methyl-4-(4chlorophenylsulphonyl)thien-2-ylcarbonyl]-4-(4-fluorobenzoyl)piperidine;
1-(thien-2-ylcarbonyl)-4-(2,4-difluorobenzoyl)piperidine;
1-(1-(4-isobutylphenyl)ethyl]-4-(benzoyl)piperidine;
1-{1-[4-(4-trifluoromethylphenoxy)phenoxy]ethyl}-4-(benzoyl)piperidine;
1-(3,5-ditrifluoromethylanilinothiocarbonyl)-4-(4-methylbenzoyl)piperidine;
1-(2-methyl-4-bromoanilinothiocarbonyl)-4-(4-methylbenzoyl)piperidine;
1-(4-fluoroanilinothiocarbonyl)-4-(4-methylbenzoyl)piperidine;
1-(thien-2-ylcarbonyl)-4-(2,4,6-trimethylbenzoyl)piperidine;
1-(cyclobutylcarbonyl)-4-(benzoyl)piperidine;
1-(2,4-dichloroanilinothiocarbonyl)-4-(4-methylbenzoyl)piperidine;
   or a pharmaceutically acceptable salt thereof;
   in the manufacture of a medicament for use in the inhibition of 11βHSD1.

In a further aspect of the invention there is provided a compound selected from Group C:
1-[2-(6-fluoro-2,3-dihydro-1,4-benzodioxin-2-yl)-2-hydroxyethyl]-4-benzoylpiperidine;
1-[2-(5-fluoro-2,3-dihydro-1,4-benzodioxin-2-yl)-2-hydroxyethyl]-4-(4-fluorobenzoyl)piperidine;
1-[3-(4-fluorophenoxy)-2-hydroxypropyl]-4-benzoylpiperidine;
1-[2-(S)-(2-(S)-5,6-difluoro-2,3-dihydro-1,4-benzodioxin-2-yl)-2-hydroxyethyl]-4-benzoylpiperidine;
1-(5-fluoro-2,3-dihydro-1,4-benzodioxin-2-ylmethyl-4-benzoylpiperidine;
1-[3-(9,10-dihydro-9,10-methanoanthracen-9-ylmethylamino)propyl]-4-(2-methoxybenzoyl)piperidine;
1-[3-(2-chloro-9,10-dihydro-9,10-methanoanthracen-9-ylmethylamino)propyl]-4-benzoylpiperidine;
1-(5-methyl-4-cyano-4-phenylhexyl)-4-(4-chlorobenzoyl)piperidine;
1-(2,4-difluorophenylsulphonyl)-4-(2,3,4,5,6-pentamethylbenzoyl)piperidine;
1-[N-(1-methyl-3-phenylpyrazol-5-yl)carbamoylmethyl]-4-(4-chlorobenzoyl)piperidine;
1-[N-(3-methyl-4-bromoisoxazol-5-ylcarbamoyl)methyl]-4-benzoylpiperidine;
1-(4,6-dimethylindol-2-ylcarbonyl)-4-(4-fluorobenzoyl)piperidine;
1-[5-(thien-2-yl)thien-2-ylcarbonyl]-4-(4-fluorobenzoyl)piperidine;
1-(t-butoxycarbonyl)-4-hydroxy-4-(2-fluorobenzoyl)piperidine;
   or a pharmaceutically acceptable salt thereof.

In a further aspect of the invention there is provided the use of a compound selected from Group D:
1-[2-(1,3-dioxo-2,4-dihydroquinazolin-2-yl)ethyl]-4-(4-fluorobenzoy)piperidine;
1-(2,3-dihydro-1,4-benzodioxin-2-ylmethyl)-4-benzoylpiperidine;
1-(2-chloro-9,10-dihydro-9,10-methanoanthracen-9-ylmethyl)-4-(pyrid-3-yl)piperidine;
1-(t-butoxycarbonyl)-4-(pyrid-3-yl)piperidine;
1-(3-nitropyrid-2-yl)-4-benzoylpiperidine;
1-(5-nitropyrid-2-yl)-4-benzoylpiperidine;
1-(5-nitropyrid-2-yl)-4-(4-fluorobenzoyl)piperidine;
1-(5-nitropyrid-2-yl)-4-(4-methylbenzoyl)piperidine;
1-(5-nitropyrid-2-yl)-4-(2,4-difluorobenzoyl)piperidine;
1-(2-nitro-4-acetylphenyl)-4-benzoylpiperidine;
1-benzylcarbonyl-4-benzoylpiperidine;
   or a pharmaceutically acceptable salt thereof;
   in the manufacture of a medicament for use in the inhibition of 11βHSD1.

Another aspect of the present invention provides a process for preparing a compound of formula (I) or a pharmaceutically acceptable salt thereof which process (wherein variable groups are, unless otherwise specified, as defined in formula (I)) comprises of:
Process 1) for compounds of formula (I) wherein X is —C(O)—; reacting an amine of formula (II): 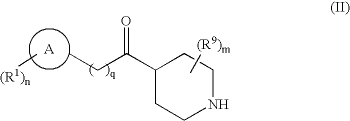
with an acid of formula (III): 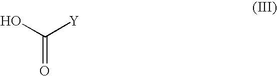
or an activated derivative thereof;
Process 2) for compounds of formula (I) wherein X is —S(O)₂—; reacting an amine of formula (II) with a sulphonyl halide of formula (IV): 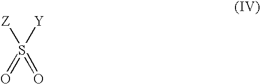
wherein Z is fluoro or chloro;
Process 3) for compounds of formula (I) wherein X is —CH₂—; reacting an amine of formula (II) with a compound of formula (V): 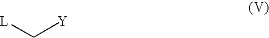
wherein L is a displaceable group; or
Process 4) for compounds of formula (I) wherein X is —CH₂—; reducing a compound of formula (I) wherein X is —C(O)—;
Process 5) for compounds of formula (I) wherein X is a direct bond; reacting an amine of formula (II) with a compound of formula (VI):
   L-Y (VI)
Process 6) for compounds of formula (I) wherein X is —C(O)NR¹¹— and R¹¹ is hydrogen; reacting an amine of formula (II) with an isocyanate of formula (VII):
   O═C═N—Y (VII)
Process 7) for compounds of formula (I) wherein X is —C(S)NR¹¹— and R¹¹ is hydrogen; reacting an amine of formula (II) with an isothiocyanate of formula (VIII):
   S═C═N—Y (VIII)
Process 8) for compounds of formula (I) wherein X is —C(O)O—; reacting an amine of formula (II) with a compound of formula (IX):
   L-C(O)—O—Y (IX)
wherein L is a displaceable group;
Process 9) for compounds of formula (I) wherein q is 0; reacting a Weinreb amide of the formula (X): 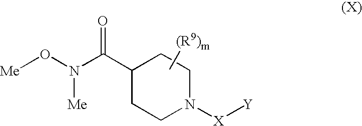
with a compound of formula (XI): 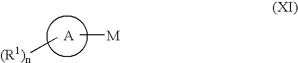
wherein M is an organometallic reagent;
Process 10) decarboxylating a compound of formula (XII): 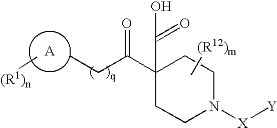
Process 11) reacting a compound of formula (XII)): 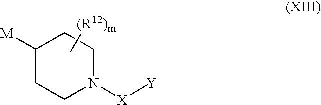
wherein M is an organometallic reagent, with a compound of formula (XIV): 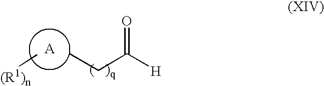
and thereafter if necessary or desirable:
i) converting a compound of the formula (I) into another compound of the formula (I);
ii) removing any protecting groups;
iii) forming a pharmaceutically acceptable salt thereof.

L is a displaceable group, suitable values for L include halo, particularly chloro or bromo, or mesyloxy.

M is an organometallic reagent, preferably a Grignard reagent, more preferably magnesium bromide.

The reactions described above may be performed under standard conditions known to the person skilled in the art. The intermediates described above are commercially available, are known in the art or may be prepared by known procedures.

It will be appreciated that certain of the various ring substituents in the compounds of the present invention may be introduced by standard aromatic substitution reactions or generated by conventional functional group modifications either prior to or immediately following the processes mentioned above, and as such are included in the process aspect of the invention. Such reactions and modifications include, for example, introduction of a substituent by means of an aromatic substitution reaction, reduction of substituents, alkylation of substituents and oxidation of substituents. The reagents and reaction conditions for such procedures are well known in the chemical art. Particular examples of aromatic substitution reactions include the introduction of a nitro group using concentrated nitric acid, the introduction of an acyl group using, for example, an acyl halide and Lewis acid (such as aluminium trichloride) under Friedel Crafts conditions; the introduction of an alkyl group using an alkyl halide and Lewis acid (such as aluminium trichloride) under Friedel Crafts conditions; and the introduction of a halogeno group. Particular examples of modifications include the reduction of a nitro group to an amino group by for example, catalytic hydrogenation with a nickel catalyst or treatment with iron in the presence of hydrochloric acid with heating; oxidation of alkylthio to alkylsulphinyl or alkylsulphonyl.

It will also be appreciated that in some of the reactions mentioned herein it may be necessary/desirable to protect any sensitive groups in the compounds. The instances where protection is necessary or desirable and suitable methods for protection are known to those skilled in the art. Conventional protecting groups may be used in accordance with standard practice (for illustration see T. W. Green, Protective Groups in Organic Synthesis, John Wiley and Sons, 1991). Thus, if reactants include groups such as amino, carboxy or hydroxy it may be desirable to protect the group in some of the reactions mentioned herein.

A suitable protecting group for an amino or alkylamino group is, for example, an acyl group, for example an alkanoyl group such as acetyl, an alkoxycarbonyl group, for example a methoxycarbonyl, ethoxycarbonyl or t-butoxycarbonyl group, an arylmethoxycarbonyl group, for example benzyloxycarbonyl, or an aroyl group, for example benzoyl. The deprotection conditions for the above protecting groups necessarily vary with the choice of protecting group. Thus, for example, an acyl group such as an alkanoyl or alkoxycarbonyl group or an aroyl group may be removed for example, by hydrolysis with a suitable base such as an alkali metal hydroxide, for example lithium or sodium hydroxide. Alternatively an acyl group such as a t-butoxycarbonyl group may be removed, for example, by treatment with a suitable acid as hydrochloric, sulphuric or phosphoric acid or trifluoroacetic acid and an arylmethoxycarbonyl group such as a benzyloxycarbonyl group may be removed, for example, by hydrogenation over a catalyst such as palladium-on-carbon, or by treatment with a Lewis acid for example boron tris(trifluoroacetate). A suitable alternative protecting group for a primary amino group is, for example, a phthaloyl group which may be removed by treatment with an alkylamine, for example dimethylaminopropylamine, or with hydrazine.

A suitable protecting group for a hydroxy group is, for example, an acyl group, for example an alkanoyl group such as acetyl, an aroyl group, for example benzoyl, or an arylmethyl group, for example benzyl. The deprotection conditions for the above protecting groups will necessarily vary with the choice of protecting group. Thus, for example, an acyl group such as an alkanoyl or an aroyl group may be removed, for example, by hydrolysis with a suitable base such as an alkali metal hydroxide, for example lithium or sodium, hydroxide. Alternatively an arylmethyl group such as a benzyl group may be removed, for example, by hydrogenation over a catalyst such as palladium-on-carbon.

A suitable protecting group for a carboxy group is, for example, an esterifying group, for example a methyl or an ethyl group which may be removed, for example, by hydrolysis with a base such as sodium hydroxide, or for example a t-butyl group which may be removed, for example, by treatment with an acid, for example an organic acid such as trifluoroacetic acid, or for example a benzyl group which may be removed, for example, by hydrogenation over a catalyst such as palladium-on-carbon.

The protecting groups may be removed at any convenient stage in the synthesis using conventional techniques well known in the chemical art.

As stated hereinbefore the compounds defined in the present invention possess 11βHSD1 inhibitory activity. These properties may be assessed using the following assay.

Assay

HeLa cells (human cervical carcinoma derived cells) were stably transfected with a construct containing four copies of the glucocorticoid response element (GRE) linked to a beta-galactosidase reporter gene (3 kb lac Z gene derived from pSV-B-galactosidase). These cells were then further stably transfected with a construct containing full-length human 11βHSD1 enzyme (in pCMVHyg) to create GRE4-βGal/11βHSD1 cells. The principal of the assay is as follows. Cortisone is freely taken up by the cells and is converted to cortisol by 11βHSD1 oxo-reductase activity and cortisol (but not cortisone) binds to and activates the glucocorticoid receptor. Activated glucocorticoid receptor then binds to the GRE and initiates transcription and translation of β-galactosidase. Enzyme activity can then be assayed with high sensitivity by colourimetric assay. Inhibitors of 11βHSD1 will reduce the conversion of cortisone to cortisol and hence decrease the production of β-galactosidase.

Cells were routinely cultured in DMEM (Invitrogen, Paisley, Renfrewshire, UK) containing 10% foetal calf serum (LabTech), 1% glutamine (Invitrogen), 1% penicillin & streptomycin (Invitrogen), 0.5 mg/ml G418 (Invitrogen) & 0.5mg/ml hygromycin (Boehringer). Assay media was phenol red free-DMEM containing 1% glutarine, 1% penicillin & streptomycin.

Compounds (1 mM) to be tested were dissolved in dimethyl sulphoxide (DMSO) and serially diluted into assay media containing 10% DMSO. Diluted compounds were then plated into transparent flat-bottomed 384 well plates (Matrix, Hudson N.H., USA).

The assay was carried out in 384 well microtitre plate (Matrix) in a total volume of 50 μl assay media consisting of cortisone (Sigma, Poole, Dorset, UK, 1 μM), HeLa GRE4-βGal/11βHSD1 cells (10,000 cells) plus test compounds (3000 to 0.01 nM). The plates were then incubated in 5% O₂, 95% CO₂ at 37° C. overnight.

The following day plates were assayed by measurement of β-galactosidase production.

A cocktail (25 μl) consisting of 10× Z-buffer (600 mM Na₂HPO₄, 400 mM NaH₂PO₄.2H₂O, 100 mM KCl, 10 mM MgSO₄.7H₂O, 500 mM β-mercaptoethanol, pH 7.0) SDS (0.2%), chlorophenol red-β-D-galactopyranoside (5 mM, Roche Diagnostics) was added per well and plates incubated at 37° C. for 3-4 hours. β-Galactosidase activity was indicated by a yellow to red colour change (absorbance at 570 nm) measured using a Tecan Spectrafluor Ultra.

The calculation of median inhibitory concentration (IC₅₀) values for the inhibitors was performed using Origin 6.0 (Microcal Software, Northampton Mass. USA). Dose response curves for each inhibitor were plotted as OD units at each inhibitor concentration with relation to a maximum signal (cortisone, no compound) and IC₅₀ values calculated. Compounds of the present invention typically show an IC₅₀<10 μM. For example the following results were obtained:

| Example | IC₅₀ |
|---|---|
| 380 | 50 nM |
| 13 | 254 nM |
| 223 | 97 nM |

According to a further aspect of the invention there is provided a pharmaceutical composition which comprises a compound of formula (Ia), (Ib), (Ic), (Id), (Ie), (If), (Ig), Group A or Group C or a pharmaceutically acceptable salt thereof or of the Examples, or a pharmaceutically acceptable salt thereof, as defined hereinbefore in association with a pharmaceutically-acceptable diluent or carrier.

The composition may be in a form suitable for oral administration, for example as a tablet or capsule, for parenteral injection (including intravenous, subcutaneous, intramuscular, intravascular or infusion) as a sterile solution, suspension or emulsion, for topical administration as an ointment or cream or for rectal administration as a suppository.

In general the above compositions may be prepared in a conventional manner using conventional excipients.

The compound of formula (I), or a pharmaceutically acceptable salt thereof, will normally be administered to a warm-blooded animal at a unit dose within the range 0.1-50 mg/kg that normally provides a therapeutically-effective dose. A unit dose form such as a tablet or capsule will usually contain, for example 1-1000 mg of active ingredient. However the daily dose will necessarily be varied depending upon the host treated, the particular route of administration, and the severity of the illness being treated. Accordingly the optimum dosage may be determined by the practitioner who is treating any particular patient.

We have found that the compounds defined in the present invention, or a pharmaceutically acceptable salt thereof, are effective 11βHSD1 inhibitors, and accordingly have value in the treatment of disease states associated with metabolic syndrome.

It is to be understood that where the term “metabolic syndrome” is used herein, this relates to metabolic syndrome as defined in 1) and or 2) or any other recognised definition of this syndrome. Synonyms for “metabolic syndrome” used in the art include Reaven's Syndrome, Insulin Resistance Syndrome and Syndrome X. It is to be understood that where the term “metabolic syndrome” is used herein it also refers to Reaven's Syndrome, Insulin Resistance Syndrome and Syndrome X.

According to a further aspect of the present invention there is provided a compound of formula (Ia), (Ib), (Ic), (Id), (Ie), (If), (Ig), Group A or Group C or a pharmaceutically acceptable salt thereof or of the Examples, or a pharmaceutically acceptable salt thereof, as defined hereinbefore for use in a method of prophylactic or therapeutic treatment of a warm-blooded animal, such as man.

Thus according to this aspect of the invention there is provided a compound of formula (Ia), (Ib), (Ic), (Id), (Ie), (If), (Ig), Group A or Group C or a pharmaceutically acceptable salt thereof or of the Examples, or a pharmaceutically acceptable salt thereof, as defined hereinbefore for use as a medicament.

According to another feature of the invention there is provided the use of a compound of the formula of formula (Ia), (Ib), (Ic), (Id), (Ie), (If), (Ig), Group A or Group C or a pharmaceutically acceptable salt thereof or of the Examples, or a pharmaceutically acceptable salt thereof, as defined hereinbefore in the manufacture of a medicament for use in the production of an 11βHSD1 inhibitory effect in a warm-blooded animal, such as man.

According to another feature of the invention there is provided the use of a compound selected from the Reference Examples, or a pharmaceutically acceptable salt thereof, as defined hereinbefore in the manufacture of a medicament for use in the production of an 11βHSD1 inhibitory effect in a warm-blooded animal, such as man.

Where production of or producing an 11βHSD1 inhibitory effect is referred to suitably this refers to the treatment of metabolic syndrome. Alternatively, where production of an 11βHSD1 inhibitory effect is referred to this refers to the treatment of diabetes, obesity, hyperlipidaemia, hyperglycaemia, hyperinsulinemia or hypertension, particularly diabetes and obesity. Alternatively, where production of an 11βHSD1 inhibitory effect is referred to this refers to the treatment of glaucoma, osteoporosis, tuberculosis, dementia, cognitive disorders or depression.

According to a further feature of this aspect of the invention there is provided a method for producing an 11βHSD1 inhibitory effect in a warm-blooded animal, such as man, in need of such treatment which comprises administering to said animal an effective amount of a compound of formula (I), or a pharmaceutically acceptable salt thereof.

According to a further feature of this aspect of the invention there is provided a method for producing an 11βHSD1 inhibitory effect in a warm-blooded animal, such as man, in need of such treatment which comprises administering to said animal an effective amount of a compound of Group B or Group C or a compound of formula (Ih), or a pharmaceutically acceptable salt thereof.

According to a further feature of this aspect of the invention there is provided a method for producing an 11βHSD1 inhibitory effect in a warm-blooded animal, such as man, in need of such treatment which comprises administering to said animal an effective amount of a compound of formula (Ia), (Ib), (Ic), (Id), (Ie), (If), (Ig), Group A or Group C or a pharmaceutically acceptable salt thereof or of the Examples, or a pharmaceutically acceptable salt thereof.

According to a further feature of this aspect of the invention there is provided a method for producing an 11βHSD1 inhibitory effect in a warm-blooded animal, such as man, in need of such treatment which comprises administering to said animal an effective amount of a compound selected from the Reference Examples, or a pharmaceutically acceptable salt thereof.

In addition to their use in therapeutic medicine, the compounds of formula (I), or a pharmaceutically acceptable salt thereof, are also useful as pharmacological tools in the development and standardisation of in vitro and in vivo test systems for the evaluation of the effects of inhibitors of 11βHSD1 in laboratory animals such as cats, dogs, rabbits, monkeys, rats and mice, as part of the search for new therapeutic agents.

The inhibition of 11βHSD1 described herein may be applied as a sole therapy or may involve, in addition to the subject of the present invention, one or more other substances and/or treatments. Such conjoint treatment may be achieved by way of the simultaneous, sequential or separate administration of the individual components of the treatment. Simultaneous treatment may be in a single tablet or in separate tablets. For example agents than might be co-administered with 11βHSD1 inhibitors, particularly those of the present invention, may include the following main categories of treatment:
1) Insulin and insulin analogues;
2) Insulin secretagogues including sulphonylureas (for example glibenclamide, glipizide) and prandial glucose regulators (for example repaglinide, nateglinide);
3) Insulin sensitising agents including PPARγ agonists (for example pioglitazone and rosiglitazone);
4) Agents that suppress hepatic glucose output (for example metformin);
5) Agents designed to reduce the absorption of glucose from the intestine (for example acarbose);
6) Agents designed to treat the complications of prolonged hyperglycaemia; e.g. aldose reductase inhibitors
7) Other anti-diabetic agents including phosotyrosine phosphatase inhibitors, glucose 6-phosphatase inhibitors, glucagon receptor antagonists, glucokinase activators, glycogen phosphorylase inhibitors, fructose 1,6bisphosphastase inhibitors, glutamine:fructose-6-phosphate amidotransferase inhibitors
8) Anti-obesity agents (for example sibutramine and orlistat);
9) Anti-dyslipidaernia agents such as, HMG-CoA reductase inhibitors (statins, eg pravastatin); PPARα agonists (fibrates, eg gemfibrozil); bile acid sequestrants (cholestyrarnine); cholesterol absorption inhibitors (plant stanols, synthetic inhibitors); ileal bile acid absorption inhibitors (IBATi), cholesterol ester transfer protein inhibitors and nicotinic acid and analogues (niacin and slow release formulations);
10) Antihypertensive agents such as, β blockers (eg atenolol, inderal); ACE inhibitors (eg lisinopril); calcium antagonists (eg. nifedipine); angiotensin receptor antagonists (eg candesartan), α antagonists and diuretic agents (eg. furosemide, benzthiazide);
11) Haemostasis modulators such as, antithrombotics, activators of fibrinolysis and antiplatelet agents; thrombin antagonists; factor Xa inhibitors; factor VIIa inhibitors); antiplatelet agents (eg. aspirin, clopidogrel); anticoagulants (heparin and Low molecular weight analogues, hirudin) and warfarin; and
12) Anti-inflammatory agents, such as non-steroidal anti-infammatory drugs (eg. aspirin) and steroidal anti-inflammatory agents (eg. cortisone).

In the above other pharmaceutical composition, process, method, use and medicament manufacture features, the alternative and preferred embodiments of the compounds of the invention described herein also apply.

### EXAMPLES

The invention will now be illustrated in the following non limiting Examples, in which standard techniques known to the skilled chemist and techniques analogous to those described in these Examples may be used where appropriate, and in which, unless otherwise stated:
(i) evaporations were carried out by rotary evaporation in vacuo and work up procedures were carried out after removal of residual solids such as drying agents by filtration;
(ii) all reactions were carried out under an inert atmosphere at ambient temperature, typically in the range 18-25° C., with solvents of HPLC grade under anhydrous conditions, unless otherwise stated;
(iii) column chromatography (by the flash procedure) was performed on Silica gel 40-63 μm (Merck);
(iv) yields are given for illustration only and are not necessarily the maximum attainable;
(v) the structures of the end products of the formula (I) were generally confirmed by nuclear (generally proton) magnetic resonance (NMR) and mass spectral techniques; magnetic resonance chemical shift values were measured in deuterated CDCl₃ (unless otherwise stated) on the delta scale (ppm downfield from tetramethylsilane); proton data is quoted unless otherwise stated; spectra were recorded on a Varian Mercury-300 MHz, Varian Unity plus-400 MHz, Varian Unity plus-600 MHz or on Varian Inova-500 MHz spectrometer unless otherwise stated data was recorded at 400 MHz; and peak multiplicities are shown as follows: s, singlet; d, doublet; dd, double doublet; t, triplet; tt, triple triplet; q, quartet; tq, triple quartet; m, multiplet; br, broad; ABq, AB quartet; ABd, AB doublet, ABdd, AB doublet of doublets; dABq, doublet of AB quartets; LCMS were recorded on a Waters ZMD, LC column xTerra MS C₈(Waters), detection with a HP 1100 MS-detector diode array equipped; mass spectra (MS) (loop) were recorded on VG Platform II (Fisons Instruments) with a HP-1100 MS-detector diode array equipped; unless otherwise stated the mass ion quoted is (M⁺);
(vi) unless further details are specified in the text, analytical high performance liquid chromatography (HPLC) was performed on Prep LC 2000 (Waters), Cromasil C₈, 7 μm, (Akzo Nobel); MeCN and de-ionised water 10 mM ammonium acetate as mobile phases, with suitable composition;
(vii) intermediates were not generally fully characterised and purity was assessed by thin layer chromatography (TLC), HPLC, infra-red (IR), MS or NM analysis;
(viii) where solutions were dried sodium sulphate was the drying agent;
(ix) where an “ISOLUTE-Si” column is referred to, this means a column containing 1 or 2 g of silica, the silica being contained in a 6 ml disposable syringe and supported by a porous disc of 54 Å pore size, obtained from International Sorbent Technology under the name “ISOLUTE”; “ISOLUTE” is a registered trade mark,
(x) the following abbreviations may be used hereinbefore or hereinafter:
DCM dichloromethane;
MeCN acetonitrile;
THF tetrahydrofuran;
HATU O-(7-azabenzotriazol-1-yl)-n,n,n′,n′-tetramethyluronium hexafluoro-phosphate;
PS-DIEA Polymer Supported-Diisopropylethylanine (From Argonaut Technologies);
DIEA Diisopropylethylamine;
PS-Trisamine Tris-(2-aminoethyl)amine polystyrene;
LHMDS Lithium bis(trimethylsilyl)amide;
TFA trifluoroacetic acid; and
EtOAc ethyl acetate.
xi) where an Isolute SCX-2 column is referred to, this means an “ion exchange” extraction cartridge for adsorption of basic compounds, i.e. a polypropylene tube containing a benzenesulphonic acid based strong cation exchange sorbent, used according to the manufacturers instructions obtained from International Sorbent Technologies Limited, Dyffryn Business Park, Hengeod, Mid Glamorgan, UK, CF82 7RJ;
xii) where an Isolute-NH2 column is referred to, this means an “ion exchange” extraction cartridge for adsorption of acidic compounds, i.e. a polypropylene tube containing a amino silane covalently bonded to a silica particle used according to the manufacturers instructions obtained from International Sorbent Technologies Limited, Dyffryn Business Park, Hengeod, Mid Glamorgan, UK, CF82 7RJ;
xiii) where Mettler Toledeo Myriad ALLEX liquid-liquid extractor is referred to this means an automated liquid liquid extraction workstation capable of separating aqueous and organic phases;
xiv) where as Isco CombiFlash Optix-10 parallel flash chromatography system is referred to this means an automated chromatography workstation capable of carrying out up to 10 purifications in parallel via flash chromatography using pre packed silica cartridges;
xv) where a “Biotage Quad3+ flash chromatography system” is referred to this means an automated chromatography workstation capable of carrying out up to 12 purifications in parallel via flash chromatography using pre packed silica cartridges, eg Si 12+M available from Biotage Inc, A Dyax Corp. Company;
xvi) where a “phase separation cartridge” is referred to this is an Isolute Phase Separator (70 ml) available from International Sorbent Technology; and
xvii) where a “reverse phase bond elute” is referred to this is a reverse phase bode elute cartridge supplied in various sizes from Varrian.

### Example 1

### 1-(4-Fluorobenzoyl)-4-(4-chlorobenzoyl)piperidine

To a stirred solution of (4-chlorophenyl)(4-piperidyl)methanone hydrochloride (187 mg, 0.72 mmol) and triethylamine (240 μl, 1.71 mmol) in DCM (3 ml) was added 4-fluorobenzoyl chloride (109 mg, 0.69 mmol). The reaction was left to stir at room temperature for one hour then transferred to a sep funnel and diluted to approximately 10 ml with DCM. This solution was washed with 2M HCl (5 ml), water (5 ml) and brine (5 ml) then dried, filtered and evaporated to yield product as a solid (70 mg, 29%). NMR (DMSO-d₆, 100° C.): 1.60 (m, 2H), 1.85 (m, 2H), 3.15 (t, 2H), 3.65 (m, 1H), 4.00 (m, 2H), 7.20 (t, 2H), 7.45 (m, 2H), 7.55 (d, 2H), 7.95 (d, 2H); m/z: 346.

### Examples 2-16 and Reference Examples 1-2

The procedure described in Example 1 was repeated using the appropriate reagent to replace the “4-fluorobenzoyl chloride” and the “(4chlorophenyl)(4-piperidyl)methanone hydrochloride” to obtain the compounds described below. In some cases a base wash was also carried out (NaHCO₃) prior to washing with brine.

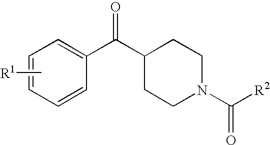

| Ex | R¹ | R² | NMR | M/z |
|---|---|---|---|---|
| 2 | 4-Cl | Cyclohexyl | 1.25(br m, 4H), 1.40-2.00(br m, | 334 |
| | | | 10H), 2.50(m, 1H), 2.80(br t, 1H), | |
| | | | 3.20(br t, 1H), 3.45(m, 1H), | |
| | | | 4.00(br m, 1H), 4.60(br m, 1H), | |
| | | | 7.45(d, 2H), 7.90(d, 2H) | |
| 3 | 4-Cl | 4-Methyl- | 0.85(br m, 1H), 1.25(s, 1H), | 342 |
| | | phenyl | 1.80(m, 4H), 2.35(s, 3H), | |
| | | | 3.10(br m, 2H), 3.50(m, 1H), | |
| | | | 7.20(d, 2H), 7.30(d, 2H), | |
| | | | 7.45(d, 2H), 7.90(d, 2H) | |
| 4 | 4-Cl | fur-2-yl | 1.80-2.00(br m, 4H), 3.20(br m, | 318 |
| | | | 2H), 3.50(m, 1H), 4.56(d, 2H), | |
| | | | 6.45(m, 1H), 7.00(d, 1H), | |
| | | | 7.45(d, 3H), 7.90(d, 2H) | |
| 5 | 4-Cl | Cyclopropyl | 0.85(m, 2H), 1.00(m, 2H), | 292 |
| | | | 1.65-2.00(br m, 5H), 2.90(br m, | |
| | | | 1H), 3.30(br m, 1H), 3.50(m, 1H), | |
| | | | 4.30(br s, 1H), 4.55(br s, 1H), | |
| | | | 7.45(d, 2H), 7.90(d, 2H) | |
| 6 | 4-F | Furan | 1.90(br m, 4H), 3.20(br m, 2H), | 302 |
| | | | 3.50(m, 1H), 4.50(d, 2H), | |
| | | | 6.50(m, 1H), 6.95(d, 1H), 7.15(t, | |
| | | | 2H), 7.50(s, 1H), 8.00(m, 2H) | |
| 7 | 4-F | Cyclohexyl | 1.30(br m, 3H), 1.40-2.00(br m, | 318 |
| | | | 11H+H2O), 2.50(m, 1H), 2.80(m, | |
| | | | 1H), 3.20(m, 1H), 3.45(m, 1H), | |
| | | | 4.00(m, 1H), 4.60(m, 1H), 7.15(t, | |
| | | | 2H), 7.95(m, 2H) | |
| 8 | 4-F | 4-Fluoro- | 1.85(br s, 4H), 3.10(br m, 2H), | 330 |
| | | phenyl | 3.50(m, 1H), 7.10(m, 4H), | |
| | | | 7.45(m, 2H), 8.00(m, 2H) | |
| 9 | 4-F | Cyclopropyl | 0.75(m, 2H), 1.00(m, 2H), | 276 |
| | | | 1.75-2.00(br m, 5H), 2.85(br m, | |
| | | | 1H), 3.30(br m, 1H), 3.50(m, 1H), | |
| | | | 4.30(br m, 1H), 4.55(br m, 1H), | |
| | | | 7.10(t, 2H), 7.95(m, 2H) | |
| RE1 | 4-Me | Thien-2-yl | DMSO-d₆: 1.50(m, 2H), 1.85(m, | 314 |
| | | | 2H), 2.35(s, 3H), 3.20(m, 2H), | |
| | | | 3.75(m, 1H), 4.30(br d, 2H), | |
| | | | 7.10(t, 1H), 7.33(d, 2H), 7.38(d, | |
| | | | 1H), 7.75(d, 1H), 7.90(d, 2H) | |
| 10 | 4-F | Thien-2-yl | 1.55(m, 2H), 1.85(m, 2H), | 318 |
| | | | 3.20(m, 2H), 3.80(m, 1H), | |
| | | | 4.30(br d, 2H), 7.10(m, 1H), | |
| | | | 7.35(m, 3H), 7.70(m, 1H), | |
| | | | 8.10(m, 2H) | |
| 11 | 4-Cl | Thien-2-yl | 1.50(m, 2H), 1.85(br d, 2H), | 334 |
| | | | 3.20(m, 2H), 3.75(m, 1H), | |
| | | | 4.30(br d, 2H), 7.10(m, 1H), | |
| | | | 7.35(d, 1H), 7.60(d, | |
| | | | 2H), 7.75(d, 1H), 8.00(d, 2H) | |
| RE2 | 4-Cl | Methyl | | 266 |
| 12 | 4-OMe | Fur-2-yl | 1.85(m, 4H), 3.10(br s, 2H), | 314 |
| | | | 3.45(m, 1H), 3.80(s, 3H), | |
| | | | 4.45(br d, 2H), 6.40(m, 1H), | |
| | | | 6.90(m, 3H), | |
| | | | 7.40(s, 1H), 7.90(d, 2H) | |
| 13 | 4-OMe | 4-Fluoro- | | 342 |
| | | phenyl | | |
| 14 | 4-OMe | Cyclopropyl | 0.75(m, 2H), 1.00(m, 2H), | 288 |
| | | | 1.75(m, 2H), 1.90(m, 3H), | |
| | | | 2.90(br s, 1H), 3.30(br s, 1H), | |
| | | | 3.50(m, 1H), 3.85(s, H), 4.30(br s, | |
| | | | 1H), 4.55(br s, 1H), 6.95(d, 2H), | |
| | | | 7.95(d, 2H) | |
| 15¹ | 4-F | 4-Fluoro- | (DMSO-d₆): 1.35(m, 2H), | 344 |
| | | benzyl | 1.75(m, 2H), 2.75(t, 1H), 3.15(t, | |
| | | | 1H), 3.65(m, 1H), 3.70(s, 2H), | |
| | | | 4.00(d, 1H), 4.40(d, 1H), 7.10(t, | |
| | | | 2H), 7.25(m, 2H), 7.35(t, 2H), | |
| | | | 8.05(m, 2H) | |
| 16 | 4-Me | 4-Fluoro- | (DMSO-d₆): 1.50(m, 2H), | 326 |
| | | phenyl | 1.80(br s, 2H), 2.35(s, 3H), | |
| | | | 3.10(br s, 2H), 3.70(m, 1H), | |
| | | | 7.25(t, 2H), 7.35(d, 2H), 7.45(m, | |
| | | | 2H), 7.90(d, 2H) | |

¹Purified by column chromatography (10 g Silica, 40% EtOAc/isohexane)

### Example 17

### 1-(5-Chlorothien-2-ylcarbonyl)4-(4-fluorobenzoyl)piperidine

To a stirred solution of 5-chlorothiophene-2-carboxylic acid (35.5 mgs, 0.2 mmol) in DCM (8 ml) was added 1-ethyl-3-(3-dimethylaminopropyl) carbodiimide hydrochloride (57.5 mgs, 0.3 mmol) and N,N diisopropylethylamine (69.7 mgs, 0.5 mmol) and the mixture was stirred for 15 mins. 4-(4-Fluorobenzoyl)piperidine hydrochloride (58 mgs, 0.24 mmol) was added and the reaction was stirred for 16 hours at room temperature. The solution was washed with 2M HCl (5 ml), saturated sodium carbonate (5 ml), water (5 ml), using a Mettler Toledeo Myriad ALLEX liquid-liquid extractor, then dried, filtered and evaporated to yield the product as a solid (33.6 mgs, 43%). M/z 351.

### Examples 18-122

The following compounds were prepared by the procedure of Example 17. “*” indicates the carbon atom that is attached to the carbonyl of formula (A).

| | |
|---|---|
| | (A) |
| 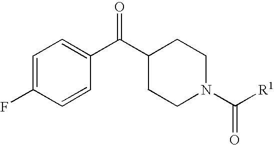 | |

| Ex | R¹ | M/z |
|---|---|---|
| 18 | 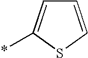 | 331 |
| 19 | 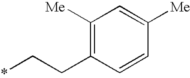 | 381 |
| 20 | 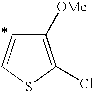 | 381 |
| 21 | 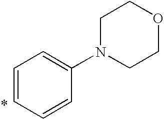 | 396 |
| 22 | 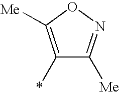 | 344 |
| 23 | 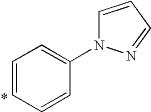 | 377 |
| 24 | 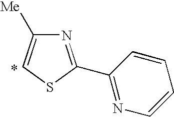 | 409 |
| 25 | 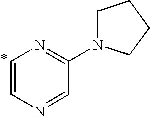 | 382 |
| 26 | 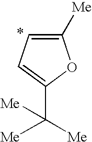 | 371 |
| 27 | 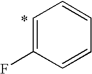 | 329 |
| 28 | 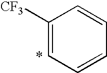 | 379 |
| 29 | 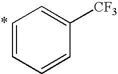 | 379 |
| 30 | 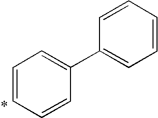 | 387 |
| 31 | 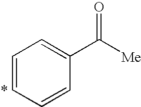 | 353 |
| 32 | 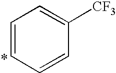 | 379 |
| 33 | 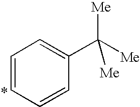 | 367 |
| 34 | 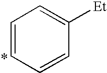 | 339 |
| 35 | 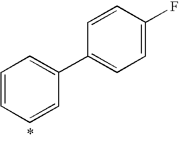 | 405 |
| 36 | 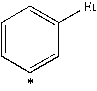 | 339 |
| 37 | 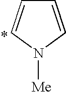 | 314 |
| 38 | 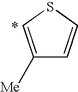 | 331 |
| 39 | 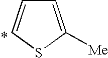 | 331 |
| 40 | 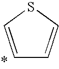 | 317 |
| 41 | 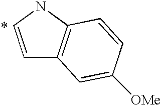 | 380 |
| 42 | 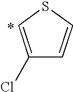 | 351 |
| 43 | 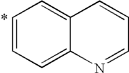 | 362 |
| 44 | 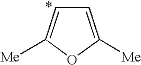 | 329 |
| 45 | 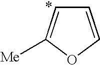 | 315 |
| 46 | 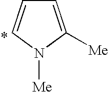 | 328 |
| 47 | 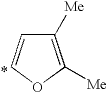 | 329 |
| 48 | 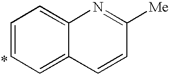 | 376 |
| 49 | 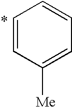 | 325 |
| 50 | 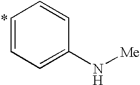 | 340 |
| 51 | 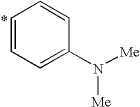 | 354 |
| 52 | 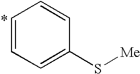 | 357 |
| 53 | 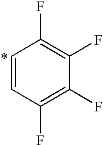 | 383 |
| 54 | 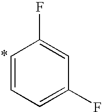 | 347 |
| 55 | 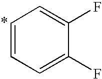 | 347 |
| 56 | 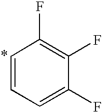 | 365 |
| 57 | 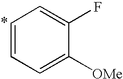 | 359 |
| 58 | 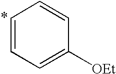 | 355 |
| 59 | 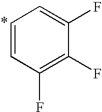 | 365 |
| 60 | 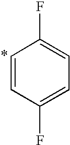 | 347 |
| 61 | 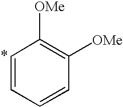 | 371 |
| 62 | 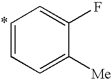 | 343 |
| 63 | 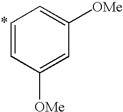 | 371 |
| 64 | 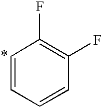 | 347 |
| 65 | 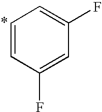 | 347 |
| 66 | 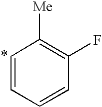 | 343 |
| 67 | 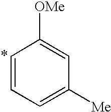 | 355 |
| 68 | 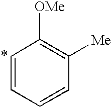 | 355 |
| 69 | 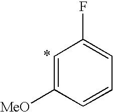 | 359 |
| 70 | 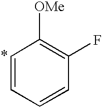 | 359 |
| 71 | 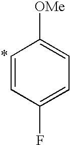 | 359 |
| 72 | 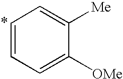 | 355 |
| 73 | 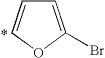 | 380 |
| 74 | 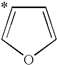 | 301 |
| 75 | 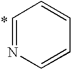 | 312 |
| 76 | 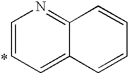 | 362 |
| 77 | 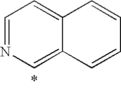 | 362 |
| 78 | 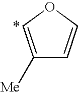 | 315 |
| 79 | 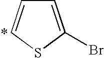 | 396 |
| 80 | 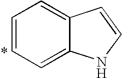 | 350 |
| 81 | 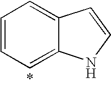 | 350 |
| 82 | 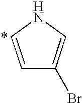 | 379 |
| 83 | 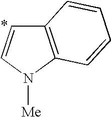 | 364 |
| 84 | 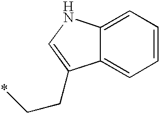 | 392 |
| 85 | 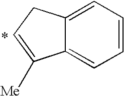 | 363 |
| 86 | 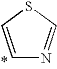 | 318 |
| 87 | 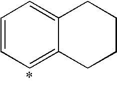 | 365 |
| 88 | 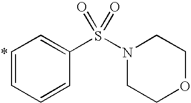 | 460 |
| 89 | 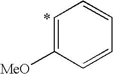 | 341 |
| 90 | 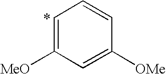 | 371 |
| 91 | 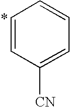 | 336 |
| 92 | 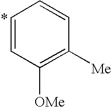 | 355 |
| 93 | 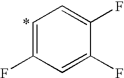 | 365 |
| 94 | 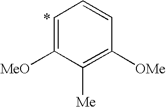 | 385 |
| 95 | 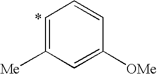 | 355 |
| 96 | 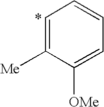 | 355 |
| 97 | 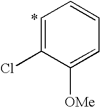 | 376 |
| 98 | 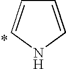 | 300 |
| 99 | 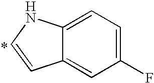 | 368 |
| 100 | 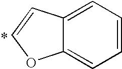 | 351 |
| 101 | 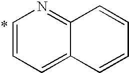 | 362 |
| 102 | 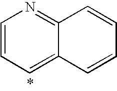 | 362 |
| 103 | 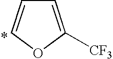 | 369 |
| 104 | 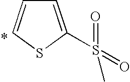 | 395 |
| 105 | 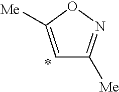 | 330 |
| 106 | 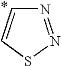 | 319 |
| 107 | 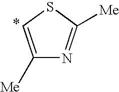 | 346 |
| 108 | 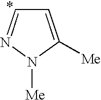 | 329 |
| 109 | 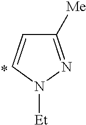 | 343 |
| 110 | 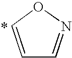 | 302 |
| 111 | 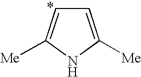 | 328 |
| 112 | 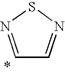 | 319 |
| 113 | 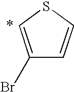 | 396 |
| 114 | 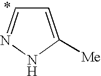 | 315 |
| 115 | 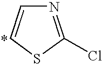 | 353 |
| 116 | 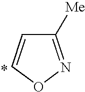 | 316 |
| 117 | 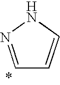 | 301 |
| 118 | 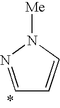 | 315 |
| 119 | 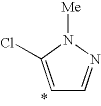 | 350 |
| 120 | 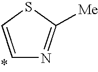 | 332 |
| 121 | 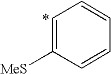 | 357 |
| 122 | 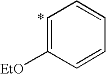 | 355 |

### Example 123

### 1-(2-Cyanobenzoyl)-4-(4chlorobenzoyl)piperidine

In a test tube was placed 2-cyanobenzoic acid (49 mg, 0.33 mmol), 4-(4-chlorobenzoyl)piperidine hydrochloride (86 mg, 0.33 mmol), N-methylmorpholine (36 μl, 0.33 mmol) and anhydrous THF (4 ml). The resulting suspension was stirred at room temperature for 15 minutes before the addition of 4-(4,6-dimethoxy-1,3,5-triazin-2-yl)-4-methylmorpholinium chloride hydrate (106 mg, 0.36 mmol). The reaction was left to stir overnight at room temperature then worked up. 1M HCl (2 ml) was added and the reaction was capped and briefly shaken then allowed to settle. The organic layer was transferred to a 4 dram vial then evaporated to yield crude product. This material was purified by prep LCMS (1-40% over 9.5 mins, MeCN/water, with a constant 5 ml/min 4% formic acid/MeCN) to yield a solid (19 mg, 16%). m/z 353.

### Examples 124-129

The procedure described in Example 123 was repeated using the appropriate reagent to replace the “2-cyanobenzoic acid” to obtain the compounds described below.

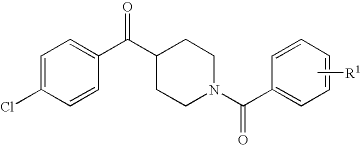

| Ex | R¹ | M/z |
|---|---|---|
| 124¹ | 3-MeO | 358 |
| 125 | 4-MeO | 358 |
| 126 | 3-CN | 353 |
| 127 | 2-MeO | 358 |
| 128 | 4-CN | 353 |
| 129 | 2,4,6-tri MeO | 418 |

¹NMR: 1.60(m, 2H), 1.90(m, 2H), 3.20(m, 2H), 3.70 9m, 1H), 3.80(s, 3H), 4.10(br s, 2H), 6.95(m, 2H), 7.00(d, 1H), 7.35(t, 1H), 7.60(d, 2H), 8.00(d, 2H)

The following General Procedures were used to make Examples 130-345 and Reference Examples 3-5.

General Procedure XX

To the acid (A) in a 2-dram glass vial was added sequentially PS-DIEA (B) and a solution of HATU (C) in DMF (D). The mixture was agitated and allowed to stand for 5-10 minutes prior to the addition of a solution of 4-(4-fluorobenzoyl)piperidine hydrochloride (E) and DIEA (F) in DMF (G). The mixture was shaken, (sonicated if required to effect dissolution) and left to stand, without agitation for 16 h. The reaction mixture was poured onto an Isolute SCX-2 column (1 g, 0.4 mmol/g) aligned over an Isolute-NH2 column (1 g, 0.6 mmol/g) transferring with DCM (0.5 ml). The columns were then eluted under atmospheric pressure with DCM (2.5 column volumes). The eluents were then evaporated in vacuo, taken up in MeCN (1 ml), an LC-MS analysis sample taken (10 μl) and evaporated again in vacuo to yield the final compound.

General Procedure YY

To the acid (A) in a 2-dram glass vial was added sequentially: PS-DIEA (B), a solution of 4-(4-fluorobenzoyl)piperidine hydrochloride (E) and DIEA (F) in DMF (G) and a solution of HATU (C) in DMF (D). The mixture was shaken, (sonicated if required to effect dissolution) and left to stand, without agitation for 16 hrs. The reaction mixture was filtered through a double fritted 6 ml reservoir, the residue was washed with DCM (0.5 ml) and the filtrated was concentrated in vacuo. The samples were purified by preparative HPLC. Preparative Reverse Phase HPLC was performed using an Xterra 19×50 mm C18 column with a water (A)/MeCN (B) gradient at 25 ml/min as typified in the following table. The eluent was modified during chromatography with a flow of a 5% solution of ammonia in MeCN (C).

| Time (mins) | A % | B % | C % |
|---|---|---|---|
| 0 | 94 | 1 | 5 |
| 1 | 94 | 1 | 5 |
| 7.5 | 0 or 45 | 95 or 50 | 5 |
| 7.51 | 0 | 100 | 0 |
| 8.5 | 0 | 100 | 0 |
| 8.51 | 94 | 1 | 5 |
| 9.5 | 94 | 1 | 5 |

General Procedure ZZ

Procedure XX was observed except that the compounds were further dissolved in EtOAc, loaded onto an Isolute-Si 1 g column and eluted with EtOAc (3 column volumes). A 15 μl analysis sample (for LC-MS) was taken from the filtrate and the remaining evaporated in vacuo to provide the desired compounds.

General Procedure AA

Procedure YY was observed except that purification was performed using the Isco CombiFlash Optix-10 parallel flash chromatography system. The evaporated samples were dissolved in EtOAc (1 ml) and loaded onto a 2 g Isolute-Si column. These were attached to the Optics-10 system over a 12 g silica column and run in one of the below methods:
i) Gradient of isohexane/EtOAc, Flow rate 30 ml/rain
   0-3 minutes 50%-100% EtOAc
   3-6 minutes 100% EtOAc
ii) Gradient of isohexane/EtOAc, Flow rate 30 ml/min
   0-5 minutes 100% EtOAc

Specific Variations of the above general Procedures are given in the following table

| Gen- | | | | | | | |
|---|---|---|---|---|---|---|---|
| eral | | | | | | | |
| Pro- | | | | | | | |
| ce- | A | B (mg) | C | D | E | F | G |
| dure | (mmols) | 3.56 mmol/g | (mmol) | (ml) | (mmol) | (mmol) | (ml) |
| XXa | 0.225 | 220 | 0.25 | 2 | 0.25 | 0.5 | 0.66 |
| XXb | 0.225 | 220 | 0.25 | 1.5 | 0.25 | 0.25 | 1 |
| XXc | 0.225 | 220 | 0.25 | 1 | 0.25 | 0.388 | 1 |
| XXd | 0.225 | 220 | 0.25 | 2 | 0.25 | 0.25 | 0.6 |
| YYa | 0.225 | 220 | 0.25 | 1.5 | 0.25 | 0.25 | 1 |
| ZZa | 0.225 | 220 | 0.25 | 1 | 0.25 | 0.388 | 1 |
| XXe | 0.3 | 220 | 0.3 | 1.5 | 0.3 | 0.33 | 1 |
| YYb | 0.3 | 220 | 0.3 | 1.5 | 0.3 | 0.33 | 1 |
| BBg | 0.45 | 220 | 0.45 | 1.5 | 0.45 | 0.45 | 1 |
| YYc | 0.45 | 440 | 0.45 | 1 | 0.5 | 0.657 | 1 |
| XXf | 0.225 | 220 | 0.225 | 1 | 0.225 | 0.338 | 1 |
| XXh | 0.3 | 260 | 0.3 | 1 | 0.3 | 0.45 | 1 |
| ZZh | 0.3 | 260 | 0.3 | 1 | 0.3 | 0.45 | 1 |
| YYf | 0.225 | 220 | 0.225 | 1 | 0.225 | 0.338 | 1 |
| BBf | 0.225 | 220 | 0.225 | 1 | 0.225 | 0.338 | 1 |
| YYh | 0.3 | 260 | 0.3 | 1 | 0.3 | 0.45 | 1 |

General Procedure BB

Procedure YY was observed except that purification was performed using a Biotage Quad3+ flash chromatography system. The evaporated samples were dissolved in DCM (1 ml) and loaded onto Biotage Si 12+M columns, which were placed in the Biotage system and chromatographed using either isohexane (25%)/EtOAc (75%) or isohexane (50%)/EtOAc (50%) depending on the polarity of the compound.

### Examples 130-345 and Reference Examples 3-5

The following compounds were prepared by the General Procedures detailed above. “*” indicates the carbon atom that is attached to the carbonyl of formula (A).

| | |
|---|---|
| | (A) |
| 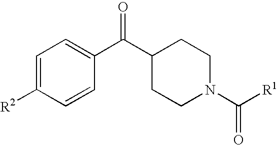 | |

| | G. | | | |
|---|---|---|---|---|
| Ex | Proc | R¹ | R² | M/z |
| 130 | XXb | 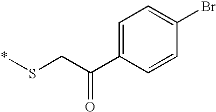 | F | 480.3 |
| 131 | XXb | 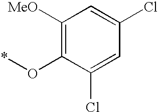 | F | 440.3 |
| 132 | XXa | 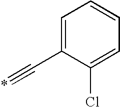 | F | 370.4 |
| 133 | XXa | 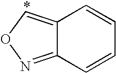 | F | 353.4 |
| 134 | XXa | 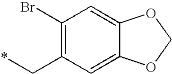 | F | 464.3 |
| 135 | YYa | 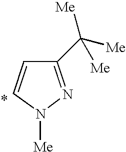 | F | 372.7 |
| 136 | XXb | 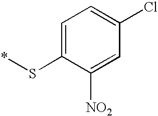 | F | 437.3 |
| 137 | XXb | 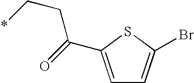 | F | 468.3 |
| 138 | YYa | 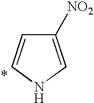 | F | 346.7 |
| 139 | YYa | 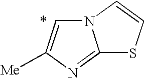 | F | 372.7 |
| 140 | YYa | 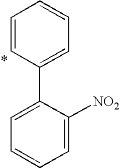 | F | 432.5 |
| 141 | YYa | 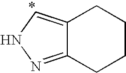 | F | 355 |
| 142 | YYa | 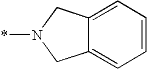 | F | 367.7 |
| 143 | XXa | 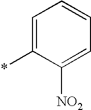 | F | 371.4 |
| 144 | XXa | 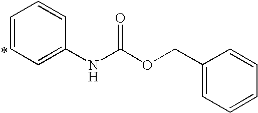 | F | 461.4 |
| 145 | YYa | 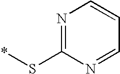 | F | 359 |
| 146 | YYa | 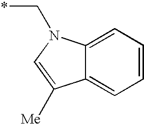 | F | 393.7 |
| 147 | XXa | 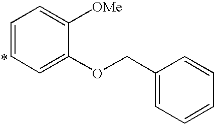 | F | 448.4 |
| RE3¹ | XXd | 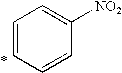 | F | 357.36 |
| 148 | XXc | 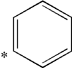 | F | 312.45 |
| 149 | XXc | 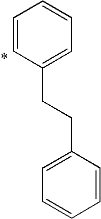 | F | 416.48 |
| 150 | XXc | 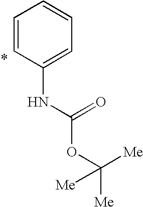 | F | 427.46 |
| 151 | XXc | 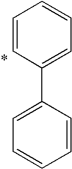 | F | 388.47 |
| 152 | XXc | 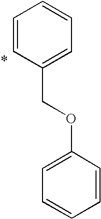 | F | 418.45 |
| 153 | XXc | 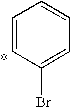 | F | 390.35 |
| 154 | XXc | 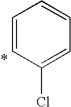 | F | 346.42 |
| 155 | XXc | 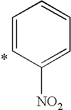 | F | 347.45 |
| 156 | XXc | 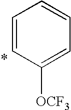 | F | 396.42 |
| 157 | XXc | 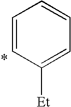 | F | 340.5 |
| 158 | ZZa | 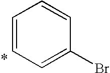 | F | 390.2 |
| 159 | ZZa | 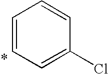 | F | 346.3 |
| 160 | ZZa | 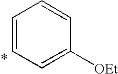 | F | 356.4 |
| 161 | ZZa | 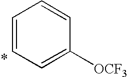 | F | 396.3 |
| 162 | ZZa | 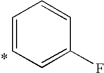 | F | 330.4 |
| 163 | ZZa | 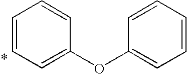 | F | 404.3 |
| 164 | ZZa | 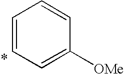 | F | 342.4 |
| 165 | ZZa | 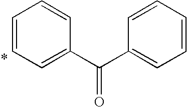 | F | 416.3 |
| 166 | ZZa | 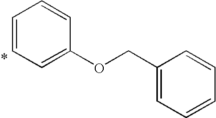 | F | 418.3 |
| 167 | ZZa | 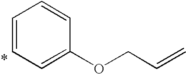 | F | 368.4 |
| 168 | ZZa | 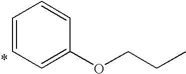 | F | 370.4 |
| 169 | ZZa | 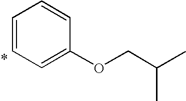 | F | 384.4 |
| 170 | ZZa | 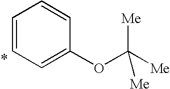 | F | 384.4 |
| 171 | XXc | 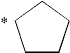 | F | 304.52 |
| 172 | XXc | 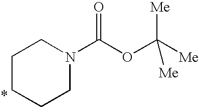 | F | 419.55 |
| 173 | XXc | i-Pr | F | 278.51 |
| 174 | XXc | Hept-3-yl | F | 334.4 |
| 175 | XXc | t-Butyl | F | 292.4 |
| 176 | XXc | 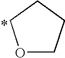 | F | 306.51 |
| 177 | XXc | 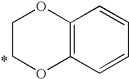 | F | 370.52 |
| 178 | XXc | Pent-3-yl | F | 306.55 |
| 179 | XXc | 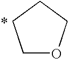 | F | 306.52 |
| 180 | XXc | 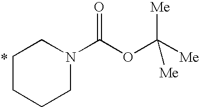 | F | 419.57 |
| 181 | XXc | 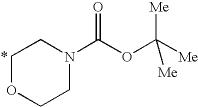 | F | 421.54 |
| 182 | XXc | 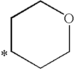 | F | 320.54 |
| 183 | XXc | 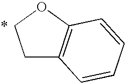 | F | 354.55 |
| 184 | XXc | 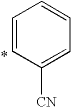 | F | 337.45 |
| 185 | XXc | 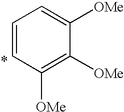 | F | 402.54 |
| 186 | ZZa | 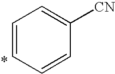 | F | 337.3 |
| 187 | ZZa | 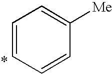 | F | 326.3 |
| 188 | ZZa | 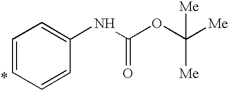 | F | 427.3 |
| 189 | ZZa | 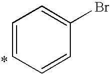 | F | 390.2 |
| 190 | ZZa | 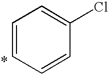 | F | 346.3 |
| 191 | ZZa | 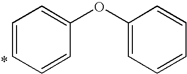 | F | 404.3 |
| 192 | ZZa | 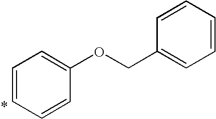 | F | 418.3 |
| 193 | ZZa | 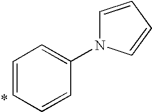 | F | 377.3 |
| 194 | ZZa | 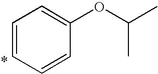 | F | 370.4 |
| 195 | ZZa | 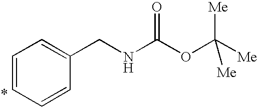 | F | 441.3 |
| 196 | ZZa | 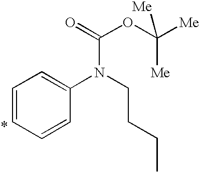 | F | 427.3 |
| 197 | ZZa | 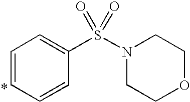 | F | 461.3 |
| 198 | ZZa | 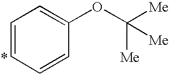 | F | 384.4 |
| 199 | XXb | *CH₂—S—C(S)—NMe₂ | F | 369.4 |
| 200 | XXb | 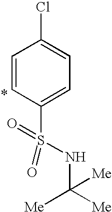 | F | 479.4 |
| 201 | YYa | 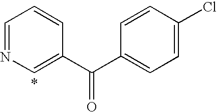 | F | 451.5 |
| 202 | YYa | 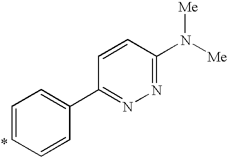 | F | 433.6 |
| 203 | XXe | 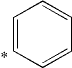 | Cl | 328.5 |
| 204 | XXe | 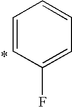 | Cl | 346.4 |
| 205 | XXe | 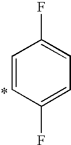 | Cl | 364.4 |
| 206 | XXe | 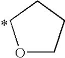 | Cl | 322.5 |
| 207 | XXe | Pent-3-yl | Cl | 322.5 |
| 208 | XXe | 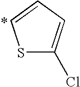 | Cl | 368.4 |
| 209 | XXe | 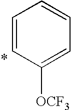 | Cl | 412.4 |
| 210 | XXe | 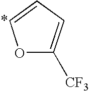 | Cl | 386.4 |
| 211 | XXe | 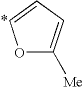 | Cl | 332.4 |
| 212 | YYb | 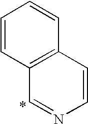 | Cl | 379.5 |
| 213 | YYb | 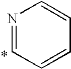 | Cl | 329.4 |
| 214 | YYb | 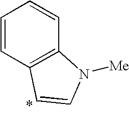 | Cl | 381.5 |
| 215 | YYb | 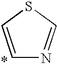 | Cl | 335.4 |
| 216 | YYb | 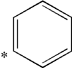 | MeO | 324.5 |
| 217 | XXe | 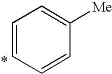 | MeO | 338.5 |
| 218 | XXe | 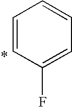 | MeO | 342.5 |
| 219 | XXe | 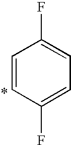 | MeO | 360.5 |
| 220 | XXe | 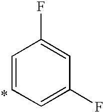 | MeO | 360.5 |
| 221 | XXe | 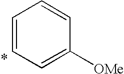 | MeO | 354.5 |
| 222 | XXe | Pent-3-yl | MeO | 318.5 |
| 223 | XXe | 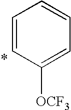 | MeO | 408.5 |
| 224 | XXe | 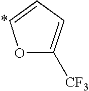 | MeO | 382.4 |
| 225 | XXe | 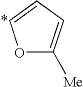 | MeO | 328.5 |
| 226 | XXe | 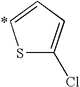 | MeO | 364.4 |
| 227 | XXe | 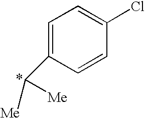 | F | 388.4 |
| 228 | XXe | 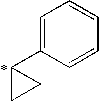 | F | 352.5 |
| 229 | XXe | 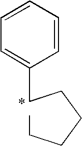 | F | 380.5 |
| 230 | XXe | 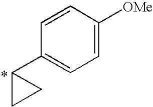 | F | 382.5 |
| 231 | XXe | 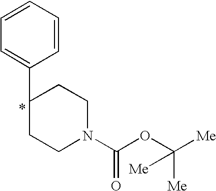 | F | 439.5 (M −t-butyl) |
| 232 | XXe | 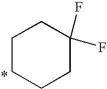 | F | 354.5 |
| 233 | XXe | *CH₂—CF₃ | F | 318.4 |
| 234 | XXe | 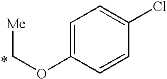 | F | 390.4 |
| 235 | ZZe | 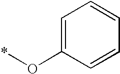 | F | 342.5 |
| 236 | XXe | 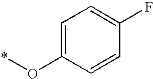 | F | 360.5 |
| 237 | XXe | 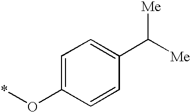 | F | 384.5 |
| 238 | ZZe | 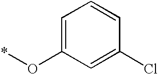 | F | 376.4 |
| 239 | XXe | 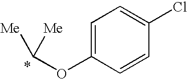 | F | 404.4 |
| 240 | XXe | 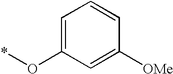 | F | 372.5 |
| 241 | ZZe | 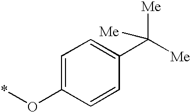 | F | 398.5 |
| 242 | ZZe | 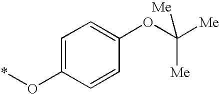 | F | 414.5 |
| 243 | XXe | 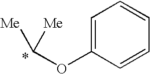 | F | 370.5 |
| 244 | ZZe | 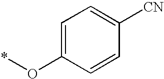 | F | 367.5 |
| 245 | ZZe | 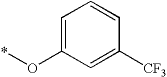 | F | 410.4 |
| 246 | ZZe | 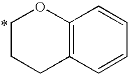 | F | 368.5 |
| 247 | ZZe | 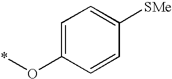 | F | 388.5 |
| 248 | XXe | 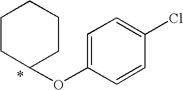 | F | 444.4 |
| 249 | XXe | 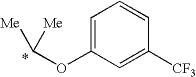 | F | 438.4 |
| 250 | ZZe | 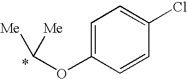 | F | 418.4 |
| 251 | XXe | 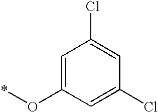 | F | 410.5 |
| 252 | XXe | 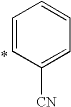 | MeO | 349.5 |
| 253 | YYb | 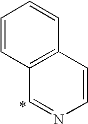 | MeO | 375.5 |
| 254 | YYb | 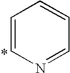 | MeO | 325.5 |
| 255 | YYb | 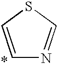 | MeO | 331.5 |
| 256 | BBg | 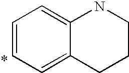 | F | 367.5 |
| 257 | BBg | 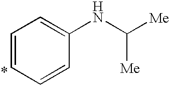 | F | 369.5 |
| 258 | XXe | 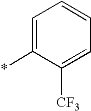 | F | 394.4 |
| 259 | XXe | 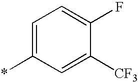 | F | 412.5 |
| 260 | XXe | 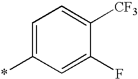 | F | 398.4 |
| 261 | XXe | 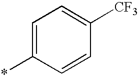 | F | 394.5 |
| 262 | XXe | 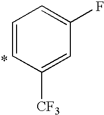 | F | 398.5 |
| 263 | XXe | 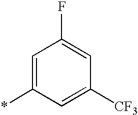 | F | 412.5 |
| 264 | XXe | *(CH₂)₂CF₃ | F | 332.5 |
| 265 | XXe | 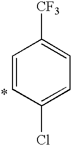 | F | 414.4 |
| 266 | XXe | 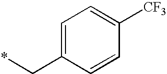 | F | 408.5 |
| 267 | XXe | 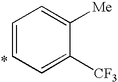 | F | 394.5 |
| 268 | XXe | *CH(Me)—CH₂—CF₃ | F | 346.5 |
| 269 | XXe | 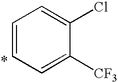 | F | 414.4 |
| 270 | XXe | 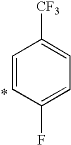 | F | 398.4 |
| 271 | YYb | 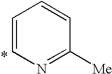 | F | 327.5 |
| 272 | YYb | 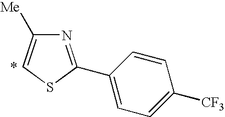 | F | 477.6 |
| 273 | YYb | 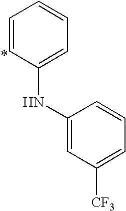 | F | 471.6 |
| 274 | YYb | 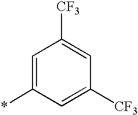 | F | 462.6 |
| 275 | YYb | 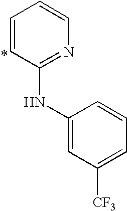 | F | 472.6 |
| 276 | YYb | 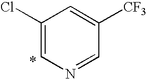 | F | 415.4 |
| 277 | YYb | 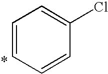 | Cl | 362.4 |
| 278 | XXe | 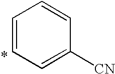 | MeO | 349.5 |
| 279 | YYb | 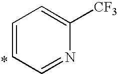 | F | 381.5 |
| 280 | YYb | 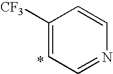 | F | 381.5 |
| 281 | XXe | 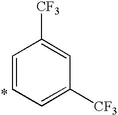 | F | 448.4 |
| 282 | YYb | 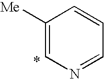 | F | 327.5 |
| 283 | YYb | 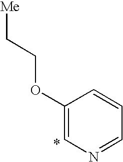 | F | 371.6 |
| 284 | ZZa | 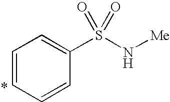 | F | 405.3 |
| 285 | ZZa | 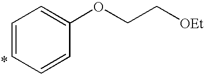 | F | 400.4 |
| RE4 | YYc | 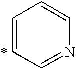 | F | 313.5 |
| 286 | YYc | 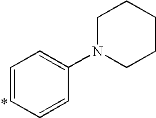 | F | 395.5 |
| 287 | XXf | 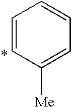 | F | 326.5 |
| 288 | XXf | 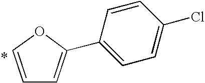 | F | 412.4 |
| 289 | XXf | 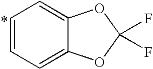 | F | 392.4 |
| 290 | XXf | 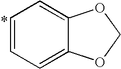 | F | 356.5 |
| 291 | XXf | 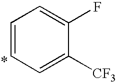 | F | 398.4 |
| 292 | XXf | 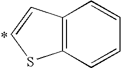 | F | 368.4 |
| 293 | XXf | 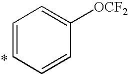 | F | 378.5 |
| 294 | XXf | 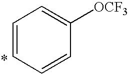 | F | 396.4 |
| 295 | XXf | 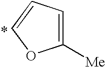 | F | 316.5 |
| 296 | XXf | 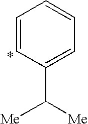 | F | 354.5 |
| RE5 | XXh | 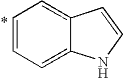 | F | 351.5 |
| 297 | XXh | 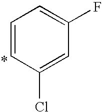 | F | 364.4 |
| 298 | XXh | 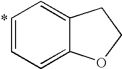 | F | 354.5 |
| 299 | XXh | 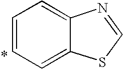 | F | 369.4 |
| 300 | XXh | 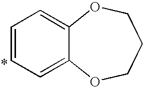 | F | 384.5 |
| 301 | XXh | 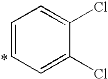 | F | 380.4 |
| 302 | XXh | 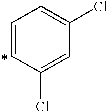 | F | 380.4 |
| 303 | XXh | 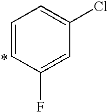 | F | 364.4 |
| 304 | ZZh | 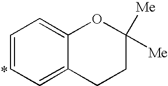 | F | 396.5 |
| 305 | XXh | 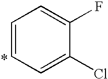 | F | 364.4 |
| 306 | XXh | 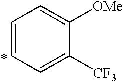 | F | 410.5 |
| 307 | XXh | 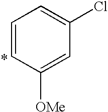 | F | 376.5 |
| 308 | XXh | 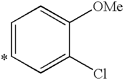 | F | 376.5 |
| 309 | XXh | 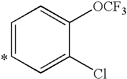 | F | 430.4 |
| 310 | XXh | 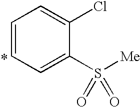 | F | 424.4 |
| 311 | XXh | 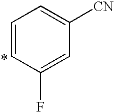 | F | 355.5 |
| 312 | XXh | 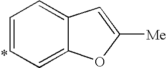 | F | 366.5 |
| 313 | YYf | 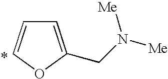 | F | 359.1 |
| 314 | YYf | 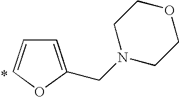 | F | 401.5 |
| 315 | BBf | 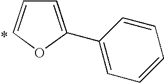 | F | 378.4 |
| 316 | YYg | 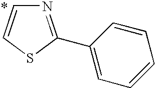 | F | 395.7 |
| 317 | YYg | 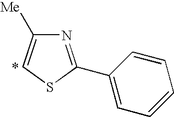 | F | 409.8 |
| 318 | YYg | 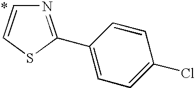 | F | 429.7 |
| 319 | YYg | 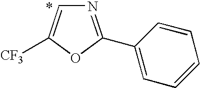 | F | 447.8 |
| 320 | YYg | 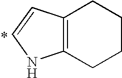 | F | 355.8 |
| 321 | YYg | 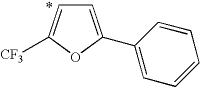 | F | 446.7 |
| 322 | YYg | 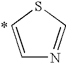 | F | 319.7 |
| 323 | XXh | 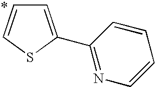 | F | 395.5 |
| 324 | XXh | 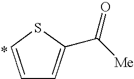 | F | 360.5 |
| 325 | XXh | 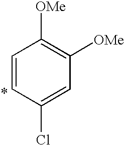 | F | 406.5 |
| 326 | XXh | 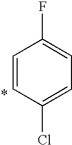 | F | 364.5 |
| 327 | XXh | 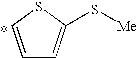 | F | 364.5 |
| 328 | XXh | 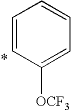 | F | 378.5 |
| 329 | XXh | 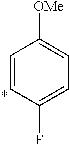 | F | 360.5 |
| 330 | XXh | 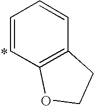 | F | 354.6 |
| 331 | XXh | 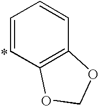 | F | 356.5 |
| 332 | XXh | 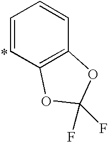 | F | 392.5 |
| 333 | XXh | 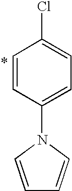 | F | 411.5 |
| 334 | XXh | 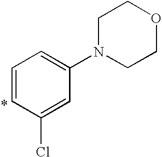 | F | 431.5 |
| 335 | YYg | *CH₂—N(Me)—C(O)—O-t-Bu | F | 279.7 (M −Boc) |
| 336 | YYg | 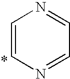 | F | 314.7 |
| 337 | YYg | 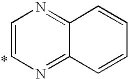 | F | 364.7 |
| 338 | YYg | 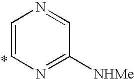 | F | 343.8 |
| 339 | XXh | 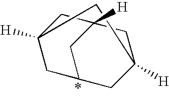 | F | 370.6 |
| 340 | XXh | 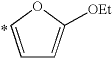 | F | 346.5 |
| 341 | YYg | 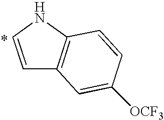 | F | 435.7 |
| 342 | YYg | 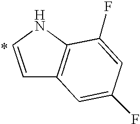 | F | 387.7 |
| 343 | YYg | 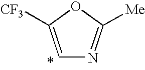 | F | 385.7 |
| 344 | YYg | 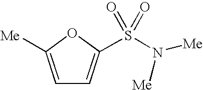 | F | 423.7 |
| 345 | YYg | 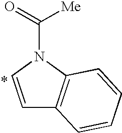 | F | 393.7 |

¹NMR(300 MHz) 1.8-2.2(4H), 3.0-3.4(2H), 3.4-4.0(2H), 4.5-4.8(1H), 7.2(2H), 7.6(2H), 8.0(2H), 8.4(2H).

### Examples 346-351

The following general procedure was used to make Examples 346-351.

To the Acid, R3-C(O)—OH, (1.83 mmol) in a 4-dram glass vial was added sequentially PS-DIEA (880 mg) and a solution of HATU (1.83 mmol) in DMF (6 ml). The mixture was agitated and allowed to stand for 5-10 minutes prior to the addition of a solution of benzoyl piperidine, (R1-Ph C(O)-piperidine), (1.83 mmol) and DEA (2.01 mmol) in DMF (6 ml). The mixture was shaken, (sonicated if required to effect dissolution) and left to stand, without agitation for 16 hours. The reaction mixture was poured onto an Isolute SCX-2 column (10 g) transferred with DCM (2 ml) and eluted with DCM (2.5 column volumes), the filtrate was then passed through and Isolute-NH2 column (20g) and eluted with DCM. The eluents were then evaporated in vacuo taken up in EtOAc and evaporated again in vacuo to give the piperidine amide. The amides (0.29 mmol) were dissolved in THF (2.5 ml) and LHMDS (0.46 ml of a 1.6 M solution in THF) added, alkylating agent (R²—Br) (1.18 mmol) was then added. The reactions were stirred at-room temperature, under argon for 19 hours and then quenched with water. The reactions mixtures were concentrated in vacuo, diluted with DCM and passed through a phase separation cartridge. The crude materials were purified using a Biotage Quad3+ flash chromatography system eluting with 25% EtOAc/isohexane to afford the final compounds.

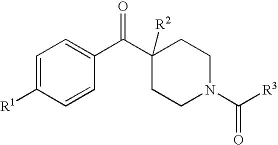

| Ex | R¹ | R² | R³ | NMR | M/z |
|---|---|---|---|---|---|
| 346 | F | Me | 4-Cl-phenyl | 7.81(2H, dd), 7.38(2H, d), | 360.4 |
| | | | | 7.30(2H, d), 7.12(2H, dd), | |
| | | | | 4.10(1H, bs), 3.23-3.11(2H, | |
| | | | | m), 2.34(2H, bs), 2.82-1.34(2H, | |
| | | | | m), 1.49(3H, s) | |
| 347 | F | Me | cyclopentyl | 7.80(2H, dd), 7.28(2H, dd), | 318.5 |
| | | | | 3.60(1H, bs), 3.30(3H, s), | |
| | | | | 3.25(1H, m), 3.12(1H, m), | |
| | | | | 2.93(1H, m), 2.10(2H, bs), | |
| | | | | 1.8-1.45(10H, m), 1.40(3H, s) | |
| 348 | F | Et | cyclopentyl | 7.80(2H, dd), 7.10(2H, dd), | 332.6 |
| | | | | 4.15(1H, bd), 3.71(1H, bd), | |
| | | | | 3.18(1H, td), 2.70-2.2.90(2H, m), | |
| | | | | 2.38(1H, bd), 2.25(1H, bd), | |
| | | | | 1.99(1H, m), 1.90-1.60(9H m), | |
| | | | | 1.60-1.49(3H, m), 0.89(3H, t) | |
| 349 | Cl | Me | cyclopentyl | 7.69(2H, d), 7.38(2H, d), 3.92(1H, | 334.5 |
| | | | | bs), 3.70-3.59(2H, m), 3.29(1H, | |
| | | | | bs), 3.05(1H, bs), 2.89(1H, m), | |
| | | | | 2.23(2H, bs), 1.90-1.67(6H, m), | |
| | | | | 1.67-1.49(4H, m), 1.45(3H, s) | |
| 350 | Cl | Pr | cyclopentyl | 7.68(2H, d), 7.38(2H, d), | 362.6 |
| | | | | 4.17(1H, bs), 3.70(1H, bs), | |
| | | | | 3.15(1H, bs), 2.91-2.72(3H, m), | |
| | | | | 2.40(1H, bd), 2.27(1H, bd), | |
| | | | | 1.92-1.61(9H, m), | |
| | | | | 1.60-1.40(5H, m) | |
| 351 | Cl | Et | cyclopentyl | 7.69(2H, d), 7.40(2H, d), | 348.5 |
| | | | | 4.15(1H, bd), 3.71(1H, bd), | |
| | | | | 3.14(1H, dd), 2.90-2.71(2H, m), | |
| | | | | 2.42(1H, bd), 2.31(1H, bd), | |
| | | | | 2.00(1H, m), 1.90-1.67(7H, m), | |
| | | | | 1.58(2H, m), 1.45(1H, dd), | |
| | | | | 0.85(3H, t) | |

### Examples 352-353

The following general procedure-was used to make Examples 352-353.

The relevant Boc protected amides (10 mg) were taken up in 1,4-dioxane (1 ml) and 4M HCl was added (1 ml). The reactions were allowed to stand at room temperature for 24 hours. The reaction mixes were then concentrated in vacuo to afford the corresponding hydrochloride salts.

| Ex | Compound | M/z | SM |
|---|---|---|---|
| 352 | 1-[4-(N-butylamino)benzoyl]-4- | 383.5 | Ex 196 |
| | (4-fluorobenzoyl)piperidine | | |
| 353 | 1-(2-aminobenzoyl)-4-(4-fluorobenzoyl)piperidine | 327.5 | Ex 150 |

### Examples 354-356 and Reference Example 6

The following general procedure was used to make Examples 354-356 and Reference Example 6.

To a solution of the acid (0.3 mmol) in DMF (1 ml) was added sequentially PS-DIEA (190 mg @ 3.56 mmol/g) and a solution of HATU (0.3 mmol) in DMF (1 ml). The mixture was allowed to stand for 5-10 minutes prior to the addition of a solution of amine (0.3 mmol) and DIEA (0.3 mmol) in DMF (1 ml). The mixture was shaken for 2 hours, then allowed to stand for 16 hours. The reaction mixture was filtered to remove PS-DIEA. The reaction mixture was poured onto an Isolute SCX-2 column (1 g, 0.4 mmol/g) aligned over an Isolute-NH₂ (1 g, 0.6 mmol/g) transferring with DCM (0.5 ml). The columns were then eluted under atmospheric pressure with DCM (2.5 column volumes). An LCMS sample was taken, then the eluents were evaporated in vacuo to yield the final compound.

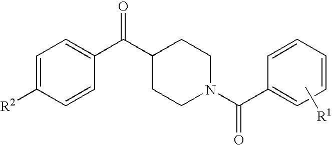

| Ex | R¹ | R² | M/z |
|---|---|---|---|
| RE6 | H | H | 294 |
| 354 | 4-i-PrO | Cl | 368 |
| 355 | 2-CN | H | 317 |
| 356 | 2-CF₃O | H | 378 |

### Example 357

### 1-(4-Methoxybenzoyl)-4-(4-fluorobenzoyl)piperidine

To paramethoxy benzoic acid (34 mg, 0.225 mmol) in a 2-dram glass vial was added a suspension of 4-(4-fluorobenzoyl)piperidine hydrochloride (0.25 mmol (60 mg), HATU (0.25 mmol, 95 mg) and DIEA (0.75 mmol, 130 μl) in THF (2m1), transferring with a further 1 ml of THF. The mixture was stirred for 19 h, filtered over Isolute SCX-2 (2×2 g) washing through with THF (1 column volume). The filtrate in turn was filtered over Isolute-NH2 (1 g) washing with THF (1 column volume). The filtrates were evaporated in vacuo to result a colourless oil. Dissolution and evaporation from methanol yielded a white solid. Yield 64.6 mg, 76.8%. NMR (300 MHz) 1.8-2.0 (4H), 3.0-3.2 (2H), 3.4-3.6 (1H), 3.9 (3H), 4-4.6 (2H), 6.9 (2H), 7.2 (2H), 7.4 (2H), 8.0 (2H); m/z 342.47.

### Example 358

### 4-(4-Trifluoromethoxybenzoyl)piperidine hydrochloride

To a suspension of Rieke Magnesium (101 mg, 4.15 mmols) in anhydrous THF (8 ml) was added a solution of 1-bromo4-(trifluoromethoxy)benzene in anhydrous THF (4 ml). The reaction was left to stand for 5 minutes then stirred for a further 5 minutes. To the resulting solution was added a solution of 1-(t-butoxycarbonyl)-4-(N-methyl-N-methoxycarbamoyl)piperidine (J. Med. Chem. 2000, 43, 21, 3895-3905; 282 mg, 1.04 mmols) in anhydrous THF (4 ml). The resulting reaction was stirred at room temperature for 30 minutes then quenched with sat NH₄Cl solution (20 ml). The reaction mixture was partitioned between water (20 ml) and EtOAc (20 ml), the layers were separated and the aqueous layer was reextracted with EtOAc (10 ml). The combined organics were washed with brine (10 ml) and dried (MgSO₄), filtered and evaporated to yield a solid. This solid was dissolved in DCM (10 ml) and treated with TFA (1.5 ml), the resulting reaction was stirred at room temperature for 1 hour then diluted to ˜20 ml and washed with 1M NaOH (20 ml) and brine (10 ml). The DCM was evaporated under reduced pressure to yield an orange oil. This oil was loaded onto an Isolute SCX-2 column which was then flushed through with MeOH, when all impurities had eluted the product was eluted off with 1% NH₃/MeOH solution. The product was dissolved in EtOH (20 ml) and treated with 1.1 eq of 1M HCl in ether. The solvent was then evaporated to yield the title compound (80 mg, 25%). M/z 274.

### Example 359

### 1-(Cyclohexylcarbonyl)-4-(4-trifluoromethoxybenzoyl)piperidine

To a stirred solution of 4-(4-trifluoromethoxybenzoyl)piperidine hydrochloride Example 358; 100 mg, 0.32 mmols) and triethylamine (82 mg, 0.81 mmols) in DCM (5 ml) was added cyclohexanecarbonyl chloride (43 mg, 0.29 mmols). The reaction was stirred at room temperature for 3 hours before washing with 1M HCl (2×3 ml), sat NaHCO₃ (3 ml) and brine. The resulting solution was then evaporated to yield the product (28 mg, 25%). M/z 384.

### Examples 360-362

The procedure described in Example 359 was repeated using the appropriate reagent to replace the “cyclohexanecarbonyl chloride” to obtain the compounds described below. The products were additionally purified by column chromatography (10 g Silica, 20 to 60% EtOAc/isohexane).

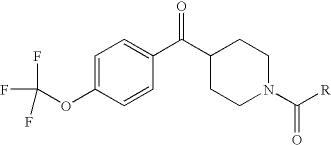

| Ex | R | NMR | M/z |
|---|---|---|---|
| 360 | Ph | NMR(DMSO-d₆): 1.60(m, 2H), 1.85(m, 2H), | 378 |
| | | 3.15(m, 2H), 3.70(m, 1H), 4.00(m, 2H), | |
| | | 7.35(m, 2H), 7.45(m, 5H), 8.10(d, 2H) | |
| 361 | 4-CN Ph | NMR(DMSO-d₆): 1.60(m, 2H), 1.85(m, 2H), | 403 |
| | | 3.15(m, 2H), 3.70(m, 1H), 4.00(m, 2H), | |
| | | 7.45(d, 2H), 7.55(d, 2H), 7.85(d, 2H), | |
| | | 8.10(d, 2H) | |
| 362 | 4-Cl Ph | NMR(DMSO-d₆): 1.60(m, 2H), 1.85(m, 2H), | 412 |
| | | 3.15(m, 2H), 3.70(m, 1H), 4.00(m, 2H), | |
| | | 7.40(d, 2H), 7.45(m, 4H), 8.10(d, 2H) | |

### Example 363

### 1-(2-Fluoro-5-methylbenzoyl)-4-(4-fluorobenzoyl)piperidine

The title compound was prepared by the procedure of Example 17. M/z 344.

### Example 364

### 1-(4-Fluorobenzoyl)-4-(3-chlorobenzoyl)piperidine

To a stirred solution of 1-(4-fluorobenzoyl)-4-(N-methyl-N-methoxycarbamoyl) piperidine (Method 2; 327 mg, 1.11 mmol) in anhydrous THF (8 ml) at 0° C. was added a 0.5M solution of 3-chlorophenyl magnesium bromide in THF (6.66 ml, 3.33 mmol). The reaction was stirred at 0° C. for ten minutes then allowed to warm to room temperature and stirred for a further 30 minutes. The reaction was quenched with sat NH₄Cl (˜20 ml) and extracted with EtOAc (2×15ml). The combined organic layers were washed with brine then dried (MgSO₄), filtered and evaporated to yield an oil. This oil was purified by column chromatography (10 g Silica, 20% EtOAc/isohexane to 40% EtOAc/isohexane) to yield a solid (55 mg, 15%). NMR (DMSO-d₆): 1.60 (m, 2H), 1.85 (m, 2H), 3.20 (t, 2H), 3.70 (m, 1H), 4.00 (m, 2H), 7.20 (t, 2H), 7.40 (m, 2H), 7.50 (t, 1H), 7.65 (m, 1H), 7.90 (m, 2H); m/z 346.

### Examples 365-376

The procedure described in Example 364 was repeated using the appropriate reagent to replace the “3-chlorophenyl magnesium bromide” to obtain the compounds described below.

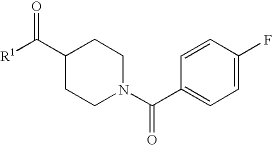

| Ex | R¹ | NMR | M/z |
|---|---|---|---|
| 365 | Benzyl | NMR(DMSO-d₆): | 326 |
| | | 1.45(m, 2H), 1.85(br s, 2H), | |
| | | 2.80(m, 1H), 2.95(br s, 2H), | |
| | | 3.85(s, 2H), 7.15(d, 2H), | |
| | | 7.30(m, 5H), 7.45(m, 2H) | |
| 366 | 4-Propyl-phenyl | NMR(DMSO-d₆): | 354 |
| | | 0.90(t, 3H), 1.60(m, 4H), | |
| | | 1.85(m, 2H), 2.65(t, 2H), 3.20(t, 2H), | |
| | | 3.70(m, 1H), 4.00(m, 2H), 7.20(t, | |
| | | 3H), 7.40(d, 2H), 7.45(m, 2H), | |
| | | 7.90(d, 2H) | |
| 367 | 2-Chloro-thien-5-yl | NMR(DMSO-d₆): 1.65(m, 2H), | 352 |
| | | 1.85(m, 2H), 2.20(t, 2H), 3.55(m, | |
| | | 1H), 4.05(m, 2H), 7.20(m, 3H), | |
| | | 7.45(m, 2H), 7.90(d, 1H) | |
| 368 | 2-Methyl-pyrid-6-yl | | 327 |
| 369 | 3-Methyl-phenyl | 1.60(m, 2H), 1.85(br d, 2H), 2.40(s, | 326 |
| | | 3H), 3.20(t, 2H), 3.70(m, 1H), | |
| | | 4.00(br d, 2H), 7.20(t, 2H), | |
| | | 7.45(m, 4H), 7.80(m, 2H) | |
| 370 | 4-t-Butyl-Phenyl | 1.30(s, 9H), 1.60(m, 2H), | 368 |
| | | 1.80(m, 2H), 3.20(m, 2H), 3.70(m, | |
| | | 1H), 4.00(m, 2H), 7.20(t, 2H), | |
| | | 7.45(m, 2H), 7.55(d, 2H), 7.90(d, | |
| | | 2H) | |
| 371 | 3-Methoxy-phenyl | 1.65(m, 2H), 1.90(m, 2H), | 342 |
| | | 3.20(m, 2H), 3.70(m, 1H), 3.85(s, | |
| | | 3H), 4.05(m, 2H), 7.25(m, 3H), | |
| | | 7.45(m, 4H), 7.60(d, 1H) | |
| 372 | 4-Phenyl-phenyl | 1.60(m, 2H), 1.90(m, 2H), | 388 |
| | | 3.20(t, 2H), 3.75(m, 1H), 4.05(br d, | |
| | | 2H), 7.20(t, 2H), 7.45(m, 5H), | |
| | | 7.70(d, 2H), 7.80(d, 2H), 8.05(d, 2H) | |
| 373 | Cyclopentyl | | 304 |
| 374 | 1,3-Benzo- | | 356 |
| | dioxol-5-yl | | |
| 375³ | 2-Methylphenyl | | 326 |
| 376 | 4-MeS phenyl | (DMSO-d₆): 1.60(m, 2H), | 358 |
| | | 1.80(m, 2H), 2.55(s, 3H), 3.20(m, | |
| | | 2H), 3.65(m, 1H), 4.00(br d, 2H), | |
| | | 7.25(t, 2H), 7.40(d, 2H), 7.45(d, | |
| | | 2H), 7.90(d, 2H) | |

¹Further purified by prep LCMS (1-40% over 9.5 mins, MeCN/water, with a constant 5 ml/min 4% formic acid/MeCN)
²Further purified by prep LCMS (9-95% over 9.5 mins, MeCN/water, with a constant 5 ml/min 4% formic acid/MeCN)
³Further purified by prep LCMS, conditions in the following table where A is water; B is MeCN; and C is 36% ammonia/MeCN. Collection was at 254 nm.

| Time (mins) | A % | B % | C % |
|---|---|---|---|
| 0 | 94 | 1 | 5 |
| 1 | 94 | 1 | 5 |
| 7.5 | 0 | 95 | 5 |
| 7.51 | 0 | 100 | 0 |
| 8.5 | 0 | 100 | 0 |
| 8.51 | 94 | 1 | 5 |
| 9.5 | 94 | 1 | 5 |

### Example 377

### 1-(4-Fluorobenzoyl)-4-(3-methoxymethylbenzoyl)piperidine

To a suspension of Rieke Mg (36 mg) in THF (1.4 ml) at room temperature, under Argon, was added a solution of (3-bromophenyl) methyl methyl ether (JACS, 1989, 111(16), 6311-20; 301 mg, 1.5 mmol). The reaction was left to stand for 10 minutes then stirred slowly for a further 5 minutes. To the resulting yellow solution was added a solution of 1-(4-fluorobenzoyl)-4-(N-methyl-N-methoxycarbamoyl) piperidine (Method 2; 150 mg, 0.51 mmol) in THF (1 ml). The reaction was stirred at room temperature for 3.5 hours then quenched with sat NH₄Cl (˜10 ml) and extracted with EtOAc (2×5 ml). The combined organics were washed with brine (5 ml) then dried (MgSO₄), filtered and evaporated to yield an oil. This oil was purified by column chromatography (20 g Silica, 20 to 60% EA/isohexane) to yield the product as a white solid (40 mg, 30%). NMR (DMSO-d₆): 1.60 (m, 2H), 1.80 (m, 2H), 3.20 (t, 2H), 3.35 (s, 3H), 3.70 (m, 1H), 4.00 (m, 2H), 4.50 (s, 2H), 7.20 (t, 2H), 7.50 (br m, 3H), 7.55 (d, 1H), 7.90 (s, 2H); m/z 356.

### Examples 378-392

The procedure described in Example 377 was repeated using the appropriate reagent to replace the “(3-Bromophenyl) methyl methyl ether” to obtain the compounds described below.

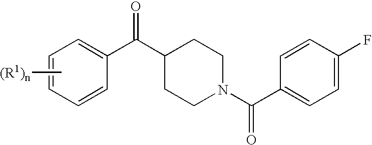

| Ex | (R¹)ₙ | NMR | M/z |
|---|---|---|---|
| 378 | 4-CF₃ | NMR (DMSO-d₆): 1.60 (m, 2H), 1.90 (m, 2H), 3.20 (m, 2H), 3.75 | 380 |
| | | (m, 1H), 4.00 (br d, 2H), 7.20 (t, 2H), 7.45 (m, 2H), 7.85 (d, 2H), | |
| | | 8.15 (d, 2H) | |
| 379 | 3-Me, | NMR (DMSO-d₆): 1.50 (m, 2H), 1.80 (m, 2H), 2.40 (s, 3H), 3.10 (br | 360 |
| | 4-Cl | s, 2H), 3.75 (m, 1H), 7.25 (t, 2H), 7.45 (m, 2H). 7.55 (d, 1H), 7.85 | |
| | | (m, 1H), 7.95 (s, 1H) | |
| 380 | 4-CF₃O | NMR (DMSO-d₆): 1.60 (m, 2H), 1.85 (m, 2H), 3.20 (m, 2H), 3.70 | 396 |
| | | (m, 1H), 4.05 (br d, 2H), 7.20 (t, 2H), 7.50 (m, 4H), 8.10 (d, 2H) | |
| 381 | 3-Cl, 4-F | NMR (DMSO-d₆): 1.55 (m, 2H), 1.85 (m, 2H), 3.20 (m, 2H), 3.70 | 364 |
| | | (m, 1H), 4.00 (m, 2H), 7.25 (m, 2H), 7.45 (m, 2H), 7.50 (m, 1H), | |
| | | 8.00 (m, 1H), 8.10 (m, 1H) | |
| 382 | 3,5-diCl | NMR (DMSO-d₆): 1.55 (m, 2H), 1.85 (m, 2H), 3.15 (t, 2H), 3.75 | 380 |
| | | (m, 1H), 4.00 (m, 2H), 7.25 (t, 2H), 7.45 (m, 2H), 7.80 (s, 1H), 7.90 | |
| | | (s, 2H) | |
| 383 | 4-i-PrO | NMR (DMSO-d₆): 1.25 (d, 6H), 1.50 (m, 2H), 1.80 (br s, 2H), 3.65 | 370 |
| | | (m, 1H), 4.75 (m, 1H), 7.00 (d, 2H), 7.25 (t, 2H), 7.45 (m, 2H), 7.95 | |
| | | (d, 2H) | |
| 384 | 3-MeO, | NMR (DMSO-d₆): 1.60 (m, 2H), 1.85 (m, 2H), 3.20 (t, 2H), 3.70 | 376 |
| | 4-Cl | (m, 1H), 3.95 (s, 3H), 4.00 (m, 2H), 7.25 (t, 2H), 7.45 (m, 2H), 7.55 | |
| | | (m, 3H) | |
| 385 | 3,4-diCl | NMR (DMSO-d₆): 1.50 (m,2H), 1.80 (brs, 2H), 3.10 (br s, 2H), | 380 |
| | | 3.75 (m, 1H), 7.25 (t, 2H), 7.45 (m, 2H), 7.80 (d, 1H), 7.95 (d, 1H), | |
| | | 8.20 (s, 1H) | |
| 386 | 3-Me, | NMR (DMSO-d₆): 1.50 (m, 2H), 1.80 (m, 2H), 2.20 (s, 3H), 3.75 | 356 |
| | 4-MeO | (m, 1H), 3.85 (s, 3H), 7.00 (d, 1H), 7.25 (t, 2H), 7.45 (m, 2H), 7.80 | |
| | | (s, 1H), 7.90 (m, 1H) | |
| 387 | 3-MeS | NMR (DMSO-d₆): 1.50 (m, 2H), 1.80 (br s, 2H), 2.50 (s, 3H), 3.10 | 358 |
| | | (br s, 2H), 3.75 (m, 1H), 7.25 (t, 2H), 7.45 (br m, 4H), 7.75 (m, 2H) | |
| 388 | 2,4-di F | | 348 |
| 389¹ | 4-Cl, 3- | NMR (DMSO-d₆): 1.60 (m, 2H), 1.80 (m, 2H), 3.10 (m, 2H), 3.65 | 466 |
| | (PhCH₂ | (m, 1H), 4.00 (br d, 2H), 4.65 (s, 2H), 4.70 (s, 2H), 7.20 (t, 2H), | |
| | OCH₂-) | 7.35 (br m, 4H), 7.45 (m, 2H), 7.60 (d, 1H), 7.90 (d, 1H), 8.10 (s, 1H) | |
| 390² | 4-i-PrS | NMR (DMSO-d₆): 1.30 (d, 6H), 1.60 (m, 2H), 1.85 (m, 2H), 3.15 | 386 |
| | | (m, 2H), 3.70 (m, 2H), 4.00 (br d, 2H), 7.20 (t, 2H), 7.45 (m, 4H), | |
| | | 7.90 (d, 2H) | |
| 391 | 3-EtO | NMR (DMSO-d₆): 1.30 (t, 3H), 1.50 (m, 2H), 1.80 (br s, 2H), 3.75 | 356 |
| | | (m, 1H), 4.10 (q, 2H), 7.25 (m, 3H), 7.45 (m, 4H), 7.55 (d, 1H) | |
| 392³ | 4-Cl-3- | NMR (DMSO-d₆): 1.60 (m, 2H), 1.85 (m, 2H), 3.20 (m, 2H), 3.40 | 390 |
| | (MeOC | (s, 3H), 3.70 (m, 1H), 4.00 (m, 2H), 4.60 (s, 2H), 7.20 (t, 2H), 7.45 | |
| | H₂-) | (m, 2H), 7.55 (d, 1H), 7.90 (d, 1H), 8.00 (s, 1H) | |

¹Startmg material: Method 10
²Startmg material: J. Med. Chem., (1998), 41(26), 5198-5218
³Startmg material: Method 11

### Example 393

### 1-(4-Fluorobenzoyl)-4-(3-trifluoromethoxybenzoyl)piperidine

A suspension of Rieke magnesium (100 mg) in THF (4 ml) was placed in a tube. To this suspension was added a solution of 1-bromo-3-(trifluoromethoxy)benzene (1 g, 4.1 mmols) in THF (2 ml). The resultant reaction was stirred at room temperature for 20 minutes before the addition of a solution of 1-(4-fluorobenzoyl)-4-(N-methyl-N-methoxy carbamoyl)piperidine (Method 2; 301 mg, 1 mmol) in THF (3 ml). The reaction was then left to stir for 2.5 hours before quenching with saturated NH₄Cl solution. The reaction was then treated with water (2 ml), capped and shaken the allowed to settle. The organic layer was decanted off and evaporated to yield an oil. This oil was purified by column chromatography (40 g Si, 20 to 100% EA/isohexane) to yield the product as a white solid (86 mg, 21%). NMR (DMSO-d₆): 1.50 (m, 2H), 1.80 (br m, 1H), 3.75 (m, 1H), 7.25 (t, 2H), 7.45 (m, 2H), 7.70 (m, 2H), 7.90 (s, 1H), 8.05 (d, 1H); m/z 396.

### Examples 394-395

The procedure described in Example 393 was repeated using the appropriate reagent to replace the “1-bromo-3-(trifluoromethoxy)benzene” to obtain the compounds described below.

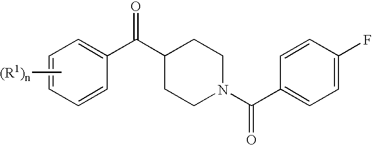

| Ex | (R¹)ₙ | NMR | M/z |
|---|---|---|---|
| 394 | 3-i-PrO | NMR (DMSO-d₆); 1.25 (d, 6H), 1.50 (m, 2H), 1.80 (m, 2H), 3.75 (m, | 370 |
| | | 1H), 4.70 (m, 1H), 7.20 (m, 1H), 7.25 (m, 2H), 7.40 (m, 4H), 7.55 (d,1H) | |
| 395 | 3-BuO | NMR (DMSO-d₆): 0.90 (t, 3H), 1.45 (m, 4H), 1.70 (m, 2H), 1.80 (br s, | 384 |
| | | 2H), 3.70 (m, 1H), 4.00 (m, 2H), 7.20 (m, 1H), 7.25 (t, 2H), 7.45 (m, | |
| | | 4H), 7.60 (m, 1H) | |

¹Starting Material: J. Med. Chem., 40, 23, 1997, 3804-3819

### Examples 396

### 1-(4-Flurobenzoyl)-4-(4-methylsulphonylbenzoyl)piperidine; and

### Example 397

### 1-(4-Fluorobenzoyl)-4-(4-methylsulphinylbenzoyl)piperidine; and

To a stirred solution of 1-(4-fluorobenzoyl)-4-(4-methylthiobenzoyl)piperidine (Example 376; 250 mg, 0.7 mmols) in THF (5 ml) was added 3-chloroperoxybenzoic acid (75%) (242 mg, 1.05 mmols). The resulting reaction was stirred at room temperature for two hours then transferred to a separating funnel. The reaction mixture was washed with 1M NaOH (3 ml), the layers were separated and the aqueous re-extracted with EtOAc (5 ml). The combined organics were washed with brine then dried (MgSO₄), filtered and evaporated to yield a solid. This solid was purified by column chromatography (5 g Si, EtOAc to 10% MeOH/EtOAc) to yield both compounds. Example 396: NMR (DMSO-d₆): 1.65 (m, 2H), 1.90 (m, 2H), 3.20 (t, 2H), 3.25 (s, 3H), 3.75 (m, 1H), 4.00 (br d, 2H), 7.25 (t, 2H), 7.45 (m, 2H); 8.05 (d, 2H), 8.15 (d, 2H); m/z 390. Example 397: NMR (DMSO-d₆): 1.60 (m, 2H), 1.90 (m, 2H), 2.80 (s, 3H), 3.20 (m, 2H), 3.75 (m, 1H), 4.00 (br d, 2H), 7.25 (t, 2H), 7.45 (m, 2H), 7.80 (d, 2H), 8.10 (d, 2H); m/z 374.

### Examples 398-400

The procedure described in Examples 396 and 397 was repeated using the appropriate reagent to replace Example 376 to obtain the compounds described below.

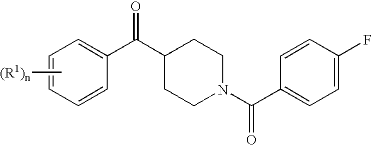

| Ex | (R¹)n | NMR | M/z | SM |
|---|---|---|---|---|
| 398 | 3- | (DMSO-d₆): 1.50 (m, 2H), 1.80 (br s, 2H), 3.80 (m, 1H), 7.25 | 390 | Ex |
| | MeSO₂ | (t, 2H), 7.45 (m, 2H), 7.85 (t, 1H), 8.20 (br d, 1H), 8.35 (br d, | | 387 |
| | | 1H), 8.40 (s, 1H) | | |
| 399 | 3-MeSO | (DMSO-d₆): 1.50 (m, 2H), 1.80 (br s, 2H), 2.80 (s, 3H), 3.80 | 374 | Ex |
| | | (m, 1H), 7.25 (t, 2H), 7.45 (m, 2H), 7.75 (t, 1H), 7.95 (d, 1H), | | 387 |
| | | 8.15 (d, 1H), 8.25 (s, 1H) | | |
| 400 | 4-iPr- | (DMSO-d₆): 1.20 (d, 6H), 1.60 (m, 2H), 1.90 (m, 2H), 3.15 | 418 | Ex |
| | S(O)₂— | (m, 2H), 3.45 (m, 1H), 3.75 (m, 1H), 4.05 (m, 2H), 7.25 (t, | | 390 |
| | | 2H), 7.50 (m, 2H), 8.00 (d, 2H), 8.20 (d, 2H) | | |
| 401 | 4-iPr- | (DMSO-d₆): 1.00 (d, 3H), 1.20 (d, 3H), 1.60 (m, 2H), 1.90 (m, | 402 | Ex |
| | S(O)- | 2H), 3.05 (m, 2H), 3.15 (m, 2H), 3.75 (m, 1H), 4.00 (m, 2H), | 390 | |
| | | 7.20 (t, 2H), 7.45 (m, 2H), 7.75 (d, 2H), 8.10 (d, 2H) | | |

### Example 402

### 1-(4-Methylbenzoyl)-4-(4-dimethylaminobenzoyl)piperidine

A vial charged with 1-(4-methylbenzoyl)-4-(4-fluorobenzoyl)piperidine (Example 187; 80 mg, 0.25 mmols), morpholine (45 mg, 0.52 mmols) and DMF (4 ml) was heated at 190° C. for 45 minutes in a microwave. The process was repeated three times and the resulting crude reaction mixtures were combined for work up and purification.-The volatiles were removed under reduced pressure and the resulting oil was purified by column chromatography (20 g Silica, 20 to 60% EtOAc/isohexane) to yield the product as a solid (118 mg, 29%). NMR (DMSO-d₆): 1.50 (m, 2H), 1.70 (br s, 2H), 2.30 (s, 3H), 3.00 (s, 6H), 3.60 (m, 1H), 6.70 (d, 2H), 7.25 (m, 4H), 7.85 (d, 2H); m/z 351.

### Example 403

### 1-(4-Methylbenzoyl)-4-(4-cyanobenzoyl)piperidine

A vial charged with 1-(4-methylbenzoyl)-4-(4-fluorobenzoyl)piperidine (Example 187; 80 mg, 0.24 mmols), KCN (16 mg, 0.24 mmols) and DMF (4 ml) was heated in a microwave at 180° C. for 55 minutes. This procedure was repeated twice then the three crude reaction mixtures were combined and evaporated under reduced pressure. The resulting orange solid was partitioned between EtOAc (30 ml) and water (30 ml), the organic layer was separated and then washed with brine (15 ml), dried (MgSO₄), filtered and evaporated to yield a gummy solid. Recrystallisation with EtOH yielded 40 mg of the title compound. The EtOH filtrate was then evaporated and the residue was purified by column chromatography (10 g Silica, 20 to 60% EtOAc/isohexane) to yield a further 46 mg of material. NMR (DMSO-d₆): 1.60 (m, 2H), 1.90 (m, 2H), 2.40 (s, 3H), 3.20 (t, 2H), 3.75 (m, 1H), 4.05 (br d, 2H), 7.30 (m, 4H), 7.90 (d, 2H), 8.10 (d, 2H); m/z 333.

### Example 404

### 1,4Bis-(4-fluorobenzoyl)-4-methylpiperidine

To a stirred solution of 1,4-bis-(4-fluorobenzoyl)piperidine (Example 8; 200 mg, 0.61 mmol) in anhyd THF (5 ml) was added a 1M solution of lithium bis(trimethyl)amide in THF (1.53 ml, 1.53 mmol). The reaction was stirred at room temperature for 15 minutes before the addition of MeI (346 mg, 2.44 mmols). The reaction was then left to stir overnight at room temperature. Water (2 ml) was added to the reaction then the volatiles were removed under reduced pressure. The product was partitioned between 1M HCl (15 ml) and DCM (20ml). The organic layer was then separated and washed with sat NaHCO₃ (15 ml) and brine (10 ml) then dried (MgSO₄), filtered and evaporated to yield an oil. This oil was purified by column chromatography (10 g Silica, 10% EtOAc/isohexane to 40% EtOAc/isohexane) to yield a solid (83 mg, 39%). NMR (DMSO-d₆): 1.40 (s, 3H), 1.65 (m, 2H), 2.10 (m, 2H), 3.35 (m, 2H), 3.60 (m, 2H), 7.25 (m, 4H), 7.45 (m, 2H), 7.80 (m, 2H); m/z 344.

### Example 405

### 3,4-Cis-1,4-Bis-(4-fluorobenzoyl)-3-methylpiperidine

To a stirred solution of 3-methyl-4-(4-fluorobenzoyl)piperidine hydrochloride (Method 4; 119 mg, 0.46 mmol) and triethylamine (140 mg, 1.39 mmol) in DCM (4 ml) was added 4-fluorobenzoyl chloride (66 mg, 0.41 mmol). The reaction was stirred at room temperature for 30 minutes then worked up. Reaction transferred to a separating funnel, diluted to 10 ml with DCM then washed with 1M HCl (2×5 ml), sat NaHCO₃ (5 ml) and brine (5 ml). The organic layer was then dried (MgSO₄), filtered and evaporated to yield a solid (101 mg, 71%). NMR (DMSO-d₆): 0.70 (d, 3H), 1.60 (m, 1H), 1.95 (m, 1H), 2.25 (m, 1H), 3.20 (m, 1H), 3.40 (m, 1 H), 3.80 (m, 2H), 3.95 (br m, 1H), 7.25 (t, 2H), 7.30 (t, 2H), 7.45 (m, 2H), 8.05 (m, 2H); m/z 344.

### Examples 406-407

The procedure described in Example 405 was repeated using the appropriate reagent to replace the “4-fluorobenzoyl chloride” to obtain the compounds described below (wherein the stereochemistry depicted in the below formula is relative rather than absolute, i.e. the compounds are the cis isomers).

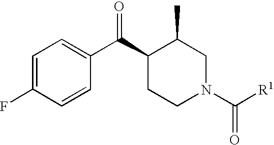

| Ex | R¹ | NMR | M/z |
|---|---|---|---|
| 406 | Cyclopropyl | NMR (DMSO-d₆): 0.70 (m, 7H), 1.60 (m, 1H), 1.90 (m, 2H), | 290 |
| | | 2.20 (m, 1H), 3.10 (br m, 1H), 3.40 (br d, 1H), 3.80 (m, 1H), | |
| | | 4.05 (m, 1H), 4.25 (m, 1H), 7.30 (t, 2H), 8.00 (m, 2H) | |
| 407 | Thien-2-yl | NMR (DMSO-d₆): 0.70 (d, 3H), 1.65 (m, 1H), 1.95 (m, 1H), | 332 |
| | | 2.30 (m, 1H), 3.30 (m, 1H), 3.50 (m, 1H), 3.90 (m, 1H), 4.10 (m, | |
| | | 1H), 4.20 (m, 1H), 7.10 (m, 1H), 7.30 (t, 2H), 7.35 (m, 1H), 7.70 | |
| | | (m, 1H), 8.10 (m, 2H) | |

### Example 408

### 1-(Thien-2-ylsulphonyl)-4-(4-chlorobenzoyl)piperidine

To a stirred solution of (4-chlorophenyl)(4-piperidyl)methanone hydrochloride (100 mg, 0.41 mmol) and triethylamine (104 mg, 1.03 mmol) in DCM (4 ml) was added 2-thiophenesulphonyl chloride (71 mg, 039 mmol). The reaction was stirred at room temperature for 1 hour then diluted to approximately 10 ml with DCM and transferred to a sep funnel. The solution was then washed with 2M HCl (5 ml), water (5 ml) and brine (5 ml), then dried, filtered and evaporated to yield the product as a solid (83 mg, 55%). NMR (DMSO-d₆): 1.55 (m, 2H), 1.90 (d, 2H), 2.55 (m, 2H), 3.50 (m, 1H), 3.65 (d, 2H), 7.30 (s, 1H), 7.50 (d, 2H), 7.60 (br s, 1H), 8.00 (d, 2H), 8.05 (m, 1H); m/z 370.

### Examples 409-426

The procedure described in Example 408 was repeated using the appropriate reagent to replace the “2-thiophenesulphonyl chloride” to obtain the compounds described below. In some cases a base wash was also carried out (NaHCO₃) prior to washing with brine.

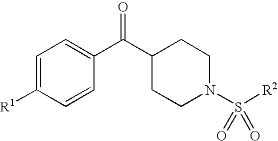

| Ex | R¹ | R² | NMR | M/z |
|---|---|---|---|---|
| 409 | F | 2-CF₃ phenyl | | 416 |
| 410 | F | 2-Br phenyl | | 426 |
| 411 | F | 3-Br phenyl | (DMSO-d₆): 1.55 (m, 2H), 1.85 (br d, 2H), 3.45 (t, | 426 |
| | | | 1H), 3.70 (br d, 2H), 7.30 (t, 2H), 7.60 (t, 1H), 7.80 | |
| | | | (d, 1H), 7.90 (s, 1H), 7.95 (d, 1H), 8.00 (m, 2H) | |
| 412 | F | 3-CF₃ phenyl | | 416 |
| 413 | F | 4-Cl phenyl | | 382 |
| 414 | F | 2-Cl, 4-CN | | 407 |
| | | phenyl | | |
| 415² | F | 3-Cl, 4-NH₂ | (DMSO-d₆): 1.55 (m, 2H), 1.85 (d, 2H), 2.40 (m, 2H), | 397 |
| | | phenyl | 3.45 (m, 1H), 3.60 (d, 2H), 6.30 (s, 2H), 6.90 (d, 1H), | |
| | | | 7.30 (t, 2H), 7.40 (d, 1H), 7.50 (s, 1H), 8.00 (m, 2H) | |
| 416 | F | 4-MeO | | 378 |
| | | phenyl | | |
| 417 | F | 4-F benzyl | 1.45 (m, 2H), 1.80 (d, 2H), 2.90 (t, 2H), 3.55 (m, 3H), | |
| | | | 4.40 (s, 2H), 7.20 (t, 2H), 7.35 (t, 2H), 7.45 (m, 2H). | |
| | | | 8.05 (m, 2H) | |
| 418 | Me | 4-F phenyl | | 362 |
| 419 | F | 4-F phenyl | | 366 |
| 420 | MeO | 4-F phenyl | | 378 |
| 421 | Cl | 4-F phenyl | 1.90 (m, 4H), 2.60 (m, 2H), 3.20 (m, 1H), 3.75 (m, | |
| | | | 2H), 7.25 (m, 2H), 7.40 (d, 2H), 7.80 (m, 4H) | |
| 422 | Cl | Iso propyl | 1.35 (d, 6H), 1.90 (m, 4H), 3.25 (m, 3H), 3.40 (m, | 330 |
| | | | 1H), 3.85 (m, 2H), 7.45 (d, 2H), 7.85 (d, 2H) | |
| 423 | Cl | Benzyl | 1.80 (br m, 4H), 2.85 (m, 2H), 3.25 (m, 1H), 3.60 (m, | |
| | | | 2H), 4.25 (s, 2H), 7.40 (br m, 7H), 7.85 (d, 2H) | |
| 424 | Cl | 4-Me phenyl | 1.90 (m, 4H), 2.45 (s, 3H), 2.55 (m, 2H), 3.10 (m, | 378 |
| | | | 1H), 3.80 (m, 2H), 7.35 (d, 2H), 7.40 (d, 2H), 7.65 (d, | |
| | | | 2H), 7.80 (d, 2H) | |
| 425 | Cl | Me | 2.00 (m, 4H), 2.85 (s, 3H), 3.00 (m, 2H), 3.35 (m, | 302 |
| | | | 1H), 3.80 (m, 2H), 7.45 (d, 2H), 7.85 (d, 2H) | |
| 426 | MeO | 4-Me phenyl | 1.90 (m, 4H), 2.45 (s, 3H), 2.55 (m, 2H), 3.15 (m, | 374 |
| | | | 1H), 3.75 (m, 2H), 3.85 (s, 3H), 6.90 (d, 2H), 7.35 (d, | |
| | | | 2H), 7.65 (d, 2H), 7.85 (d, 2H) | |

¹Product purified by column chromatography (10 g Silica, 40% EtOAc/isohexane) to yield white solid.
²The sulphonylchloride used was 4-acetamido-3-chlorobenzenesulfonyl chloride, the acetyl group was removed during the reaction/work up.

### Example 427

### 1-(3-Chlorophenylsulphonyl)-4-(4-fluorobenzoyl)piperidine

To a stirred solution of 4-(4-fluorbenzoyl)piperidine hydrochloride (51 mg, 0.21 mmol) and triethylamine (52 mg, 0.51. mmol) in DCM (8 ml) was added 3-chlorobenzenesulfonyl chloride (40 mgs, 0.19 mmol) The reaction was stirred at room temperature for 16 hours. The solution was then washed with 2M HCl (5 ml), saturated sodium carbonate (5 ml) and water (5 ml) using a Mettler Toledeo Myriad ALLEX liquid-liquid extractor then dried, filtered and evaporated to yield the product as a solid (58.8 mgs, 62.4%). M/z 382.

### Examples 428-456

The procedure described in Example 427 was repeated using the appropriate reagents to obtain the compounds described below.

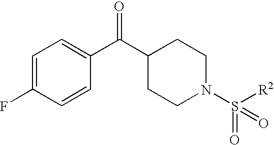

| Ex | R² | M/z |
|---|---|---|
| 428 | 2,5-Dimethylphenyl | 375 |
| 429 | 2-Chloro-6-methylphenyl | 396 |
| 430 | 5-Fluoro-2-methylphenyl | 379 |
| 431 | 2-Methylphenyl | 361 |
| 432 | 2-Chlorophenyl | 382 |
| 433 | 2,5-Dichlorothien-3-yl | 422 |
| 434 | 2-Fluorophenyl | 365 |
| 435 | 2,4,5-Trifluorophenyl | 401 |
| 436 | 3-Fluorophenyl | 365 |
| 437 | 3,5-Dimethylisoxazol-4-yl | 366 |
| 438 | 2-Cyanophenyl | 372 |
| 439 | 2-Nitro-4-methoxyphenyl | 422 |
| 440 | 4-Ethylphenyl | 375 |
| 441 | 2-Chloro-4-flurophenyl | 400 |
| 442 | 2-Methoxy-5-methylphenyl | 391 |
| 443 | 3-Methoxyphenyl | 377 |
| 444 | 2,4-Difluorophenyl | 383 |
| 445 | Thien-3-yl | 353 |
| 446 | 3-Methylphenyl | 361 |
| 447 | 5-Chloro-1,3-dimethylpyrazol-4-yl | 400 |
| 448 | Butyl | 327 |
| 449 | 4-Bromophenyl | 426 |
| 450 | Isopropyl | 313 |
| 451 | 4-Methylphenyl | 361 |
| 452 | 4-Trifluoromethylphenyl | 415 |
| 453 | 4-Acetamidophenyl | 404 |
| 454 | 2-Chlorothien-5-yl | 388 |
| 455 | 2,6-Diflurophenyl | 383 |
| 456 | Ethyl | 299 |

### Example 457

### 1-(4-Fluorophenylsulphonyl)-4-(3-methoxybenzoyl)piperidine

To a stirred solution of 1-(4-fluorophenylsulphonyl)-4-(N-methyl-N-methoxycarbamoyl)piperidine (Method 8; 250 mg, 0.76 mmol) in anhydrous THF (5 ml) at 0° C. was added a 1M solution of 3-methoxyphenylmagnesium bromide in THF (2.66 ml, 2.66 mol). The reaction was stirred at 0° C. for ten minutes then allowed to warm temperature and stirred for a further 30 minutes. The reaction was quenched with sat NH₄Cl solution then extracted with EtOAc (2×15 ml). The organic layers were combined, washed with brine (10 ml), dried (MgSO₄), filtered and evaporated to yield an oil. This oil was purified by column chromatography (10 g Silica, 20% EtOAc/isohexane to 40% EtOAc/isohexane) to yeild a white solid (115 mg, 40%). NMR (DMSO-d₆): 1.60 (m, 2H), 1.90 (m, 2H), 2.70 (m, 2H), 3.50 (m, 1H), 3.70 (m, 2H), 3.85 (s, 3H), 7.20 (m, 1H), 7.50 (m, 5H), 7.85 (m, 2H); m/z 378.

### Examples 458-464

The procedure described in Example 457 was repeated using the appropriate reagent to replace the “3-methoxyphenylmagnesium bromide” to obtain the compounds described below.

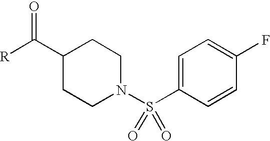

| Ex | R | NMR | M/z |
|---|---|---|---|
| 458 | 3-Me | (DMSO-d₆): 1.60 (m, 2H), 1.90 (m, 2H), 2.40 (s, 3H), 2.70 (t, 2H), | 362 |
| | phenyl | 3.45 (m, 1H), 3.70 (m, 2H), 7.45 (m, 4H), 7.70 (m, 2H), 7.90 (m, | |
| | | 2H) | |
| 459¹ | 2-Me | (DMSO-d₆): 1.60 (m, 2H), 1.85 (m, 2H), 2.30 (s, 3H), 2.65 (m, 2H), | 362 |
| | phenyl | 3.20 (m, 1H), 3.60 (m, 2H), 7.25 (m, 2H), 7.35 (m, 1H), 7.40 (m, | |
| | | 2H), 7.55 (d, 1H), 7.80 (m, 2H) | |
| 460 | 2-MeO | (DMSO-d₆): 1.60 (m, 2H), 1.90 (m, 2H), 2.65 (m, 2H), 3.20 (m, | 378 |
| | phenyl | 1H), 3.65 (m, 2H), 3.80 (s, 3H), 7.00 (t, 1H), 7.15 (d, 1H), 7.45 (m, | |
| | | 4H), 7.80 (m, 2H) | |
| 461 | 3,5-di F | 1.50 (m, 2H), 1.85 (br d, 2H), 2.45 (m, 2H), 3.45 (m, 1H), 3.65 (d, | 384 |
| | phenyl | 2H), 7.50 (m, 3H), 7.65 (m, 2H), 7.85 (m, 2H) | |
| 462³ | 2,4-di F | 1.50 (m, 2H), 1.95 (m, 2H), 2.35 (m, 2H), 2.55 (m, 1H), 3.60 (d, | 398 |
| | Benzyl | 2H), 3.85 (s, 2H), 7.00 (m, 1H), 7.15 (m, 1H), 7.25 (m, 1H), 7.50 (t, | |
| | | 3H), 7.85 m, 2H) | |
| 463² | 2-Me, 4-F | 1.55 (m, 2H), 1.85 (m, 2H), 2.30 (s, 3H), 2.60 (m, 2H), 3.20 (m, | 380 |
| | phenyl | 1H), 3.65 (m, 2H), 7.10 (m, 2H), 7.40 (t, 2H), 7.70 (m, 1H), 7.85 (m, | |
| | | 2H) | |
| 464² | 2,4-di Me | 1.55 (m, 2H), 1.85 (m, 2H), 2.30 (d, 6H), 2.65 (m, 2H), 3.20 (m, | 376 |
| | phenyl | 1H), 3.60 (m, 2H), 7.05 (m, 2H), 7.40 (t, 2H), 7.50 (d, 1H), 7.85 (m, | |
| | | 2H) | |

The material recovered from the initial chromatography was purified by prep LCMS (1-40% over 9.5 mins, MECN/water, with a constant 5 ml/min 4% formic acid /MeCN).
The material recovered from the initial chromatography was purified by prep LCMS (5-95% over 9.5 mins, MeCN/water, with a constant 5 ml/min 4% formic acid / MeCN).
The product was purified by an EtOAc recrystallization.

### Examples 465-466

The procedure described in Example 457was repeated using the appropriate reagent to replace the “3-methoxyphenylmagnesium bromide” and 1-(isopropylsulphonyl)-4-(N-methyl-N-methoxycarbamoyl)piperidine (Method 9) to obtain the compounds described below.

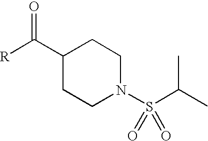

| Ex | R | NMR | M/z |
|---|---|---|---|
| 465 | 3,5-di F | (DMSO-d₆): 1.20 (d, 6H), 1.50 (m, 2H), 1.85 (br d, 2H), 3.05 (t, | 332 |
| | phenyl | 2H), 3.30 (m, 1H), 3.65 (m, 3H), 7.55 (m, 1H), 7.65 (m, 2H) | |
| 466 | 2,4 di F | 1.20 (d, 6H), 1.45 (m, 2H), 1.90 (br d, 2H), 2.70 (m, 1H), 2.95 (t, | 346 |
| | benzyl | 2H), 3.30 (m, 2H), 3.65 (br d, 2H), 3.90 (s, 2H), 7.00 (m, 1H), 7.15 | |
| | | (m, 1H). 7.25 (m, 1H) | |

### Example 467

### 1-(4-Fluorophenylsulphonyl)-4-(3-fluorobenzoyl)piperidine

To a stirred solution of 1-(4-fluorophenylsulphonyl)-4-(N-methyl-N-methoxy carbamoyl)piperidine (Method 8; 36 mg, 0.11 mmol) in anhydrous THF (1 ml) was added a 0.5M solution of 3-flurophenyl magnesium bromide in THF (0.78 ml, 0.39 mmol). The reaction was stirred at room temperature for 3 hours then quenched with sat NH₄Cl solution. Water (1 ml) and EtOAc (3 ml) were added and the reaction was capped and briefly shaken then allowed to settle. The organic layer was transferred to a weighed vial then evaporated to yield crude product. This was purified by prep LCMS to yield a gum (9 mg, 20%). M/z 366.

### Examples 468-474

The procedure described in Example 467 was repeated using the appropriate reagent to replace the “3-flurophenyl magnesium bromide” to obtain the compounds described below.

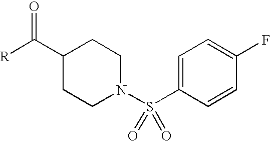

| Ex | R | M/z |
|---|---|---|
| 468 | 4-t-Butylphenyl | 404 |
| 469 | 1,3-Benzodioxol-5-yl | 392 |
| 470 | 6-Methylpyrid-2-yl | |
| 471¹ | 4-propyphenyl | 390 |
| 472 | 5-Chlorothie-2-yl | 388 |
| 473 | Pyrid-2-yl | 349 |
| 474 | Thien-2--yl | 354 |

¹NMR (DMSO-d₆): 0.85 (t, 3H), 1.55 (m, 4H), 1.80 (br d, 2H), 2.60 (t, 2H), 3.40 (m, 1H), 3.65 (m, 2H), 7.30 (d, 2H), 7.50 (t, 2H), 7.85 (m, 4H)

### Example 475

### 1-(4-Fluorophenylsulphonyl)-4-(4-fluorobenzoyl)-4-ethylpiperidine

To a stirred solution of 1-(4fluorophenylsulphonyl)-4-(4-fluorobenzoyl)piperidine (Example 419; 200 mg, 0.55 mmol) in anhydrous THF (5 ml) at 0° C. was added a 1M solution of lithium bis(trimethyl)amide in THF (1.1 ml, 1.1 mmol). The reaction was allowed to stir briefly before the addition of ethyl iodide (171 mg, 1.1 mmol). The reaction was then allowed to warm to room temperature and left to stir overnight. The volatiles were removed under reduced pressure and the resulting gummy solid was partitioned between water and EtOAc. The organic layer was separated then washed with brine, dried (MgSO₄), filtered and evaporated to yield an oil. This oil was purified by column chromatography (20 g Silica, 10% EtOAc/isohexane to 40% EtOAc/isohexane) to yield a white solid (16 mg, 7%). NMR (DMSO-d₆): 0.70 (t, 3H), 1.65.(m, 2H), 1.85 (q, 2H), 2.25 (br d, 2H), 2.40 (m, 2H), 3.35 (m, 2H), 7.25 (t, 2H), 7.50 (t, 2H), 7.70 (m, 2H1), 7.80 (m, 2H); m/z 394.

### Example 476

### 1-(Thien-2-ylmethyl)-4-(4-chlorobenzoyl)piperidine

To a stirred suspension of (4chlorophenyl)(4-piperidyl)methanone hydrochloride (200 mg, 0.82 mmol) in THF (6 ml) was added 2-thiophene carboxaldehyde (101 mg, 0.90 mmol). The reaction was stirred at 35° C. for 5 hours before the addition of sodium triacetoxyborohydride (434 mg, 2.05 mmol). The reaction was left to stir at 35° C. for 48 hours before quenching by the addition of water (10 ml). Volatiles removed under reduced pressure and the resulting solid was partitioned between water and DCM. The DCM layer was separated off and the aqueous was reextracted with DCM. The organic phases were combined and washed with brine, then -dried, filtered and evaporated to yield crude product. This crude product was dissolved in DCM and treated with PS-trisamine (60 mg) and PS-tosylchloride (290 mg) for 12 hours. The polymer bound reagents were filtered off and the solvent was removed to yield the product (98 mg, 38%). NMR: 1.85 (m, 4H), 2.00 (m, 2H), 3.00 (m, 2H), 3.20 (m, 1H), 3.75 (s, 2H), 6.95 (m, 2H), 7.25 (m, 1H), 7.40 (d, 2H), 7.85 (d, 2H).

### Example 477

### 1-(Benzyl)-4-(4-bromobenzoyl)piperidine

To a stirred solution of ethyl-N-benzyl isonipecotate (5.7 g, 24.2 mmol) in methanol (60 ml) was added a 1M solution of NaOH (60 ml, 60 mmol). The resulting mixture was stirred for 4 hours. The solution was neutralised by the addition of 2M HCl solution (30 ml, 60 mmol) then the solvent was removed in vacuo. The residue was triturated with THF (3×100 ml), the triturates were combined and evaporated to give 4.12 g of N-benzylisonipecotic acid which was used without further purification. The N-benzylisonipecotic acid (3.94 g, 18.0 mmol) was suspended in THF (100 ml) under Argon then cooled to −78° C. A 2M solution of lithium diisopropylamide was then added dropwise with stirring (22.5 ml, 45 mmol). The reaction was then allowed to warm to room temperature followed by refluxing under argon for a further hour (oil bath temperature 50° C.). This solution was then allowed to cool back to room temperature. In a separate flask 4-bromobenzoyl chloride (5.93 g, 27 mmol) was dissolved in THF (100 ml) and cooled to −78° C. The dianion solution was added dropwise to the acid chloride solution over 30 minutes. The reaction mixture was stirred at −78° C. for a further 30 minutes then allowed to warm to room temperature over night. The reaction was quenched by the addition of 2M HCl (36 ml, 72 mmol) in 100 g of crushed ice. The product was extracted with 3×200 ml DCM, dried over MgSO₄ and then evaporated to give a brown oil. Flash column chromatography was performed, eluting with 0 to 5% MeOH in DCM. 1.7 g of pure material was obtained as an orange solid. M/z 358.

### Example 478

### 1-(Pyrimidin-2-yl)-4-(4-fluorobenzoyl)piperidine

A solution of 4-(4-flurobenzoyl)piperidine hydrochloride (300 mg, 1.23 mmol), 2-chloropyrimidine (141 mg, 1.23 mmol) and triethylamine (261 mg, 2.58 mmol) in EtOH (10 ml) was stirred at reflux for 5 hours. The reaction was then cooled to room temperature and the solvent was removed under reduced pressure. The crude product was partitioned between EtOAc (20 ml) and water (20 ml). The organic layer was separated, washed with brine (10 ml) then dried (MgSO₄), filtered and evaporated to yield crude product. This material was purified by column chromatography (DCM eluent) to yield the product as an oil which crystallised on standing (123 mg, 35%). No (DMSO-d₆): 1.50 (m, 2H), 1.83 (br d, 2H), 3.10 (m, 2H), 3.75 (m, 1H), 4.65 (br d, 2H), 6.60 (t, 1H), 7.35 (t, 2H), 8.10 (m, 2H), 8.30 (d, 2H); m/z 286.

### Example 479

### 1-(4-Trifluoromethylphenyl)-4-(4-fluorobenzoyl)piperidine

Copper iodide (10 mg, 0.05 mmol), K₃PO₄ (636 mg, 3 mmol) and 4-(4-fluorobenzoyl)piperidine hydrochloride (292 mg, 1.2 mmol) were put into a glass tube. The tube was sealed with a subaseal and evacuated and back filled with Argon. This Argon purge was repeated three times. Isopropanol (1 ml), ethylene glycol (111 μl) and 4-iodobenzotrifluoride (272 mg, 1 mmol) were then added by syringe. The reaction was warmed to 75° C. and left to stir at this temperature over night. The reaction was cooled to room temperature and partitioned between water (10 ml) and ether (15 ml). The layers were separated and the aqueous layer was reextracted with ether. The combined organic layers were washed with brine, dried (MgSO₄), filtered and evaporated to yield an oil. This oil was purified by column chromatography (10 g Silica, eluting with 10% EtOAc/isohexane to 40% EtOAc/isohexane) to yield a solid (54 mg, 15%). NMR (DMSb-d₆): 1.60 (m, 2H), 1.85 (br d, 2H), 3.00 (t, 2H), 3.70 (m, 1H), 3.90 (br d, 2H), 7.05 (d, 2H), 7.35 (t, 2H), 7.45 (d, 2H), 8.10 (m, 2H); m/z 352.

### Examples 480-483

The procedure described in Example 479 was repeated using the appropriate reagent to replace the “4-iodobenzotrifluoride” to obtain the compounds described below. In cases where the “iodo” compound was a solid it was added at the start of the reaction prior to the Argon purge.

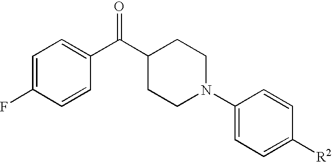

| Ex | R² | NMR | M/z |
|---|---|---|---|
| 480 | MeO | (DMSO-d₆): 1.75 (m, 2H), 1.90 (br d, 2H), 2.85 (m, 2H), 3.55 | 314 |
| | | (m, 3H), 3.70 (s, 3H), 6.80 (d, 2H), 6.90 (d, 2H), 7.30 (t, 2H), | |
| | | 8.05 (m, 2H) | |
| 481 | MeC(O)NH— | (DMSO-d₆): 1.65 (m, 2H), 1.85 (br d, 2H), 2.00 (s, 3H), 2.80 | 341 |
| | | (m, 2H), 3.55 (m, 1H), 1.60 (br d, 2H), 6.85 (d, 2H), 7.40 (m, | |
| | | 4H), 8.10 (m, 2H), 9.65 (s, 1) | |
| 482 | F | (DMSO-d₆): 1.65.(m, 2H), 1.85 (br d, 2H), 2.80 (m, 2H), 3.55 | 302 |
| | | (m, 1H), 3.60 (br d, 2H), 6.95 (m, 2H), 7.00 (t, 2H), 7.35 (t, | |
| | | 2H), 8.10 (m, 2H) | |
| 483 | MeC(O)— | (DMSO-d₆): 1.60 (m, 2H), 1.85 (br d, 2H), 2.40 (s, 3H), 3.10 | 326 |
| | | (m, 2H), 3.70 (m, 1H), 4.00 (br d, 2H), 7.00 (d, 2H), 7.35 (t, | |
| | | 2H), 7.80 (d, 2H), 8.10 (m, 2H) | |

### Example 484

### 1-(Pyrid-4-yl)-4-(4-methoxybenzoyl)piperidine

To a stirred suspension of 1-(pyrid-4-yl)-4-(carboxy)piperidine (10.31 g, 50 mmol) in DCM (200 ml) at 4° C., was added oxalyl chloride (13 ml, 151.3 mmol) and DMF (cat). The mixture was allowed to warm to ambient temperature and stirred for 18 hours. Volatile material was removed by evaporation to give a solid. This solid was added slowly to a stirred mixture of aluminium chloride (40.0 g, 300 mmol) and anisole (40 ml, 368 mmol). The mixture was heated to 85° C. and stirred for 3 hours, then allowed to cool to ambient temperature and stirred for a further 16 hours, The mixture was poured onto an ice/water mix. This was extracted with DCM (400 ml). The extract was washed with water (150 ml), brine (50 ml), water (2×200 ml) and dried over MgSO₄. Volatile material was removed by evaporation to leave a solid, which was purified by flash chromatography, eluting with 5-10% methanol in DCM to give a solid. This was recrystallized from ethanol to give the title compound (0.839 g) a solid. NMR (d₆-DMSO): 1.55 (m, 2H), 1.78 (m, 2H), 3.00 (t, 2H), 3.68 (m, 1H), 3.83 (s, 3H), 3.94 (m, 2H), 6.80 (d, 2H), 7.03 (d, 2H), 7.98 (d, 2H), 8.10 (d, 2H), MS: (ESP⁺) m/z 297.0.

### Example 485

### 1-(6-Chloronaphth-2-ylmethyl)-4-(4-fluorobenzoyl)piperidine

A solution containing 2-chloro-6-chloromethylnaphthalene (European Journal of Medicinal Chemistry (1984), 19(3), 205-14; 0.11 g; 0.5 mmol) in DMF (3 ml) was added to 4-(4-fluorobenzoyl)piperidine hydrochloride (weighed at 0.5 mmol) in DMF (3 ml). Solid potassium carbonate was added and the mixture stirred at 100° C. for 3 hours. After cooling, the mixture-was evaporated to approx. 1 ml and water (7 ml) was added. The solid products were collected by filtration and washed with water (1 ml). Yield 90%. M/z 382.2.

### Example 486

### 1-(4-Fluoroanilinothiocarbonyl)-4-(4-fluorobenzoyl)piperidine

To a stirred solution of 4-(4-fluorobenzoyl)piperidine hydrochloride (300 mg, 1.22 mmol) and triethylamine (134 mg, 1.32 mmol) in DCM (6 ml) was added 4-fluorophenyl isothiocyanate (170 mg, 1.1 mmol). The reaction was left to stir at room temperature for 15 minutes then worked up The reaction was transferred to a separating funnel and diluted to approximately 5 ml with DCM. The DCM was washed with 1M HCl (10 ml), water (10 ml) and brine (5 ml) then dried (MgSO₄), filtered and evaporated to yield a solid (300 mg, 68%). NMR (DMSO-d₆): 1.50 (m, 2H), 1.85 (br d, 2H), 3.30 (t, 2H), 3.70 (m, 1H), 4.75 (br d, 2H), 7.10 (t, 2H), 7.30 (m, 2H), 7.35 (t, 2H), 8.10 (m, 2H), 9.25 (s, 1H); m/z 361.

### Example 487

### 1-(Phenoxycarbonyl)-4-(4-fluorobenzoyl)piperidine

To a stirred suspension of 4-(4-fluorobenzoyl)piperidine hydrochloride (244 mg, 1 mmol) in DCM (10 ml) was added PS-DEA, 3.66 mmol/g, 683 mg. The reaction was stirred for 15 minutes, then phenyl chloroformate (188 mg, 1.2 mmol) was added. The reaction was stirred for 16 hours. PS-Trisamine (3.75 mmol/g, 133 mg) was added, and stirring was continued for a further hour before filtration through a PTFE phase separating membrane. The product was purified by flash column chromatography (10 g Silica), eluting 25% EtOAc in isohexane, and isolated as a white solid (118 mg, 36%). NMR (MSO-d₆): 1.40-1.70 (br s, 2H), 1.86 (d, 2H), 3.00-3.20 (br m,2H), 3.71 (m, 1H), 4.0-4.3 (br d, 2H), 7.10 (d, 2H), 7.20 (t, 1H), 7.36 (t, 4H), 8.10 (m, 2H). M/z 391.47 (M+MeCN+Na)⁺.

### Examples 488-493 and Reference Examples 7 and 8

Using the procedure given for Example 487, the following Examples were synthesised substituting the phenyl chloroformate with the appropriate chloroformate reagent.

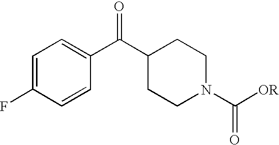

| Ex | R | NMR |
|---|---|---|
| 488 | Me | (DMSO-d₆): 1.40 (qd, 2H), 1.76 (d, 2H), 2.97 (t, 2H), 3.58 (s, 3H), |
| | | 3.59-3.68 (m, 1H), 3.98 (d, 2H), 7.34 (t, 2H), 8.02-8.15 (m, 2H) |
| RE7 | Et | (DMSO-d₆): 1.17 (t, 3H), 1.40 (qd, 2H), 1.76 (d, 2H), 2.96 (t, 2H), |
| | | 3.54-3.70 (m, 1H), 3.91-4.10 (m, 4H), 7.34 (t, 2H), 8.00-8.12 (m, |
| | | 2H) |
| 489 | Allyl | (DMSO-d₆): 1.42 (qd, 2H), 1.78 (d, 2H), 2.99 (t, 2H), 3.57-3.71 (m, |
| | | 1H), 4.01 (d, 2H), 4.51 (d, 2H), 5.21 (dd, 2H), 5.84-6.00 (m, 1H), |
| | | 7.34 (t, 2H), 8.00-8.13 (m, 2H) |
| 490 | MeOCH₂CH₂— | (DMSO-d₆): 1.41 (qd, 2H), 1.77 (d, 2H), 2.97 (t, 2H), 3.25 (s, 3H), |
| | | 3.50 (t, 2H), 3.57-3.71 (m, 1H), 3.99 (d, 2H), 4.10 (t, 2H), 7.34 (t, |
| | | 2H), 8.00-8.13 (m, 2H) |
| RE8 | Benzyl | (DMSO-d₆): 1.43 (qd, 2H), 1.78 (d, 2H), 3.01 (t, 2H), 3.56-3.72 (m, |
| | | 1H), 4.03 (d, 2H), 5.07 (s, 2H), 7.24-7.46 (m, 7H), 8.01-8.15 (m, 2H) |
| 491 | Isopropyl | (DMSO-d₆): 1.17 (d, 6H), 1.39 (qd, 2H), 1.75 (d, 2H), 2.94 (t, 2H), |
| | | 3.55-3.71 (m, 1H), 3.98 (d, 2H), 4.69-4.85 (m, 1H), 7.34 (t, 2H), |
| | | 8.01-8.12 (m, 2H) |
| 492 | 4-Fluorophenyl | (DMSO-d₆): 1.41-1.69 (br s, 2H), 1.85 (d, 2H), 2.95-3.25 (b m, 2H), |
| | | 3.64-3.80 (m, 1H), 3.97-4.29 (br d, 2H), 7.11-7.25 (m, 4H), 7.36 (t, |
| | | 2H), 8.03-8.17 (m, 2H) |
| 493 | 4-Methoxy | (DMSO-d₆): 1.40-1.70 (br s, 2H), 1.84 (d, 2H), 2.90-3.25 (br s, 2H), |
| | phenyl | 3.61-3.79 (m, 4H), 3.93-4.28 (br s, 2H), 6.89 (d, 2H), 7.03 (d, 2H), |
| | | 7.36 (t, 2H), 8.01-8.17 (m, 2H) |

### Example 494

### 1-(4-Fluoroanilinocarbonyl)-4-(4-fluorobenzoyl)piperidine

To a stirred solution of 4-(4-fluorobenzoyl)piperidine hydrochloride (200 mg, 0.82 mmol) and triethylamine (87 mg, 0.86 mmol) in DCM (4 ml) was added 4-fluorophenyl isocyanate (101 mg, 0.74 mmol). The reaction was left to stir at room temperature for 15 minutes then worked up. Reaction transferred to a separating funnel and diluted to approximately 5 ml with DCM. The DCM was washed with 1M HCl (10 ml), water (10 ml) and brine (5 ml) then dried (MgSO₄), filtered and evaporated to yield a solid (153 mg, 54%). NMR (DMSO-d₆): 1.50 (m, 2H), 1.80 (br d, 2H), 2.95 (t, 2H), 3.65 (m, 1H), 4.10 (br d, 2H), 7.05 (t, 2H), 7.35 (t, 2H), 7.45 (m, 2H), 8.10 (m, 2H), 8.50 (s, 1H); m/z 345.

### Examples 495-515 and Reference Examples 9 and 10

The procedure described in Example 494 was repeated using the appropriate reagents to replace the “4-(4-fluorobenzoyl)piperidine hydrochloride,” and “4-fluorophenyl isocyanate” to obtain the compounds described below.

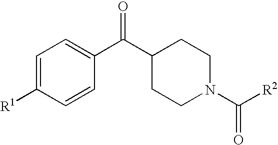

| Ex | R¹ | R² | NMR | M/z |
|---|---|---|---|---|
| 495 | 6-Bromo | Me₂N— | 1.25 (m, 2H), 1.73 (d, 2H), 2.70 (s, 6H), 2.80 | 531 |
| | naphth-2- | | (t, 2H), 3.53 (m, 3H), 7.82 (d, 1H), 7.97 (d, | |
| | yl | | 1H), 8.15 (m, 6H), 8.36 (s, 1H), 8.78 (s, 1H) | |
| | sulphonyl | | | |
| 496 | 6-Bromo | H₂N— | 1.33 (m, 2H), 1.70 (d, 2H), 2.80 (t, 2H), 3.57 | 503 |
| | naphth-2- | | (m, 1H), 3.90 (d, 2H), 5.87 (s, 2H), 7.82 (d, | |
| | yl | | 1H), 7.97 (d, 1H), 8.15 (m, 6H), 8.36 (s, 1H), | |
| | sulphonyl | | 8.78 (s, 1H) | |
| 497 | Cl | Me₂N— | 1.40-1.58 (m, 2H), 1.70-1.80 (br d, 2H), 2.73 | 295.43 |
| | | | (s, 6H), 2.78-2.94 (br t, 2H), 3.50-3.63 (br d, | |
| | | | 3H), 7.55-7.62 (d, 2H), 7.97-8.03 (d, 2H) | |
| 498 | F | (i-Pr)₂N— | | 355.53 |
| 499 | F | Piperidin-1-yl | | 319.50 |
| 500 | Cl | Anilino | 1.40-1.62 (m, 2H), 1.73-1.90 (br d, 2H), 2.90- | 343.42 |
| | | | 3.08 (app t, 2H), 3.58-3.75 (m, 1H), 4.06-4.24 | |
| | | | (br d, 2H), 7.85-7.98 (pp t, 1H), 7.15-7.30 (app | |
| | | | t, 2H), 7.38-7.53 (app d, 2H), 7.56-7.68 (app d, | |
| | | | 2H), 7.96-8.10 (app d, 2H), 8.40-8.55 | |
| RE | F | Me₂N— | 1.40-1.68 (m, 2H), 1.68-1.90 (br d, 2H), 2.58- | 279.46 |
| 9 | | | 3.0 (m, 8H), 3.50-3.75 (m, 3H), 7.28-7.50 (m, | |
| | | | 2H), 8.0-8.22 (m, 2H) | |
| RE | F | 3-Chloroanilino | | 361.42 |
| 10 | | | | |
| 501 | F | Benzylamino | | 341.8 |
| 502 | F | Anilino | | 279.42 |
| 503 | F | 2-Fluoroanilino | 1.41-1.62 (m, 2H), 1.74-1.90 (d, 2H), 2.93-3.10 | 345.45 |
| | | | (t, 2H), 3.59-3.75 (m, 1H), 4.03-4.20 (d, 2H), | |
| | | | 7.0-7.23 (m, 3H), 7.30-7.50 (m, 3H), 8.0-8.15 | |
| | | | (m, 2H), 8.17-8.30 (s, 1H) | |
| 504 | F | 3,4- | | 363.45 |
| | | Difluoroanilino | | |
| 505 | F | Morpholino | 1.40-1.59 (m, 2H), 1.70-1.82 (br d, 2H), 3.84- | 321.47 |
| | | | 2.97 (app br t, 2W), 3.03-3.17 (m, 4H), 3.50- | |
| | | | 3.70 (m, 7H), 7.27-7.40 (app t, 2H), 8.00-8.13 | |
| | | | (m, 2H) | |
| 506 | F | 3-Methylanilino | | 341.47 |
| 507 | F | 2-Ethylanilino | 1.11 (t, 3H), 1.49 (q, 2H), 1.71-1.84 (br d, 2H), | |
| | | | 2.54 (q, 2H), 2.99 (t, 2H), 3.60-3.75 (m, 1H), | |
| | | | 4.02-4.17 (br d, 2H), 7.02-7.23 (br m, 4H), | |
| | | | 7.36 (t, 2H), 7.98 (s, 1H), 8.09 (t, 2H) | |
| 508 | F | 3-Methyl | 1.41 (q, 2H), 1.66-1.82 (br d, 2H), 2.27 (s, 3H), | |
| | | benzylamino | 2.88 (t, 2H), 3.55-3.67 (m, 1H), 3.92-4.09 (br | |
| | | | d, 2H), 4.19 (d, 2H), 6.92-7.09 (m, 4h), 7.16 (t, | |
| | | | 1H), 7.34 (t, 2H), 8.08 (t, 2H) | |
| 509 | F | 2-Fluoro | 1.32-1.53 (m, 2H), 1.68-2.25 (br d, 2H), 2.89 | |
| | | benzylamino | (t, 2H), 3.54-3.68 (m, 1H), 3.94-4.07 (br d, | |
| | | | 2H), 4.27 (d, 2H), 7.01 (t, 1H), 7.06-7.19 (m, | |
| | | | 2H), 7.21-7.44 (m, 3H), 8.02-8.13 (m, 2H) | |
| 510 | F | 3-Fluoro | 1.33-1.53 (m, 2H), 1.68-1.82 (br d, 2H), 2.90 | |
| | | benzylamino | (t, 2H), 3.55-3.69 (m, 1H), 3.95-4.09 (br d, | |
| | | | 2H), 4.23 (d, 2H), 6.92-7.15 (m, 3H), 7.26-7.40 | |
| | | | (m, 3H), 8.02-8.13 (m, 2H) | |
| 511 | F | 2- | 1.40-1.57 (m, 2H), 1.72-1.85 (br d, 2H), 3.00 | 395.47 |
| | | Trifluoromethyl | (t, 2H), 3.61-3.74 (m, 1H), 4.02-4.14 (br d, | |
| | | anilino | 2H), 7.30-7.44 (m, 4H), 7.56-7.69 (m, 2H), | |
| | | | 8.04-8.13 (m, 2H), 8.17 (s, 1H) | |
| 512 | F | 2,6-Dimethyl | 1.40-1.59 (m, 2H), 1.70-1.85 (brd, 2H), 2.13 | 355.53 |
| | | anilino | (s, 6H), 3.00 (t, 2H), 3.62-3.77 (m, 1H), 4.05- | |
| | | | 4.12 (br d, 2H), 7.01 (app s, 3H), 7.35 (t, 2H), | |
| | | | 7.82 (s, 1H), 8.09 (app t, 2H) | |
| 513 | F | 2,5-Difluoro | 1.39-1.59 (m, 2H), 1.72-1.86 (br d, 2H), 3.01 | 361.43 |
| | | anilino | (t, 2H), 3.59-3.74 (m, 1H), 4.03-4.17 (br d, | (M − H)⁻ |
| | | | 2H), 6.80-6.93 (m, 1H), 7.14-7.26 (m, 1H), | |
| | | | 7.29-7.45 (m, 3H), 8.02-8.14 (m, 2H), 8.38 (s, | |
| | | | 1H) | |
| 514 | F | 4-Methoxy | 1.31-1.50 (m, 2H), 1.65-1.78 (br d, 2H), 2.86 | 371.51 |
| | | benzylamino | (t, 2H), 3.51-3.67 (m, 1H), 3.71 (s, 3H), 3.94- | |
| | | | 4.06 (br d, 2H), 4.14 (d, 2H), 6.84 (d, 2H), | |
| | | | 6.90-7.01 (m, 1H), 7.16 (d, 2H), 7.34 (t, 2H), | |
| | | | 8.02-8.12 (m, 2H) | |
| 515 | F | (R)-α-Methyl | 1.29-1.49 (m, 5H), 1.64-1.79 (br d, 2H), 2.84 | |
| | | benzylamino | (t, 2H), 3.51-3.67 (m, 1H), 3.98-4.12 (br d, | |
| | | | 2H), 4.75-4.90 (m, 1H), 6.68-6.76 (br d, 1H), | |
| | | | 7.11-7.22 (m, 1H), 7.21-7.40 (m, 6H), 8.00- | |
| | | | 8.12 (m, 2H) | |

### Example 516

### 1-[4-(Pyrid-2-yl)anilinocarbonyl]-4-(4-fluorobenzoyl)piperidine

To a stirred suspension of 4-(2-pyridyl)aniline (172 mg, 1.01 mmol) and PS-DIEA (2 mmol) in DCM (5 ml) was added trichloroacetyl chloride (134 μl, 1.2 mmol). The solutions were stirred for 72 hours. The reaction was filtered and the filtrate evaporated in vacuo. The residue was dissolved in DMSO (3 ml), and treated with sodium carbonate (424 mg, 4 mmol) and 4-fluorobenzoylpiperidine (approx 1 mmol dissolved in 2 ml DMSO) at 80° C. for 6 hours. The reaction mixture was cooled to room temperature, and evaporated under high vacuum. The resultant gum was triturated with EtOAc (10 ml) and filtration afforded the product as an off-white solid (135 mg, 33%). NMR (DMSO-d₆): 1.41-1.61 (m, 2H), 1.73-1.88 (br d, 2H), 3.01 (t, 2H), 3.59-3.77 (m, 1H), 4.08-4.25 (br d, 2H), 7.18-7.28 (app t, 1H), 7.36 (t, 2H), 7.57 (d, 2H), 7.73-7.90 (m, 2H), 7.96 (d, 2H), 8.03-8.15 (m, 2H), 8.59 (d, 1H), 8.66 (s, 1H); m/z 371.51.

### Example 517

### 1-(N-methyl-4-fluoroanilinocarbonyl)-4-(4-fluorobenzoyl)piperidine

To a stirred solution of triphosgene (297 mg, 1.0 mmol) in DCM, was added the 4-(4-fluorobenzoyl)piperidine hydrochloride (293 mg, 1.2 mmol) and DIEA (383 μl, 2.2 mmol) in one portion. The reaction was left to stir at room temperature for 30 minutes prior to adding the 4-fluoro-N-methylaniline (126 mg, 1.0 mmol). The reaction mixture was stirred at room temperature overnight then worked up. The reaction was transferred to a separating funnel and diluted to approximately 5 ml with DCM. The DCM was washed with 2M HCl (10 ml), water (10 ml) and brine (5 ml) then dried (MgSO₄), filtered and evaporated to yield a solid (65 mg, 18%). NMR (DMSO-d₆): 1.2-1.38 (m, 2H), 1.60 (br d, 2H), 2.75 (t, 2H), 3.03 (s, 3H), 3.58 (m, 1H), 3.70 (br d, 2H), 7.16 (d, 4H), 7.35 (t, 2H), 8.0 (dd, 2H); m/z 359.

### Examples 518-521

The following compounds were prepared by the procedure of Example 517.

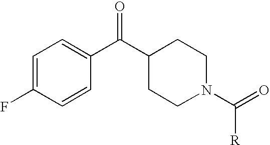

| Ex | R | NMR | M/z |
|---|---|---|---|
| 518 | 4-(4-fluorobenzoyl) | 1.41-1.58 (m, 2H), 1.73 (d, 2H), 2.90 (t, 2H), 3.6 | 441 |
| | piperidin-1-yl | (d, 6H), 7.35 (t, 4H), 8.05 (dd, 4H) | |
| 519 | 2,6-difluoroanilino | 1.41-1.58 (m, 2H), 1.80 (d, 2H), 3.0 (t. 2H), 3.6- | 363; 361 |
| | | 3.72 (m, 1H), 4.10 (d, 2H), 7.08 (d, 2H), 7.21-7.30 | (M − H)⁻ |
| | | (m, 1H), 7.31-7.40 (t, 2H), 8.04 (d, 2H) | |
| 520 | 2,3-difluoroanilino | | 363; 361 |
| | | | (M − H)⁻ |
| 521 | N-methylanilino | (DMSO-d₆): 1.27 (dt, 2H), 1.58 (br d, 2H), 2.75 (t, | 341 |
| | | 2H), 3.07 (s, 3H), 3.48 (t, 1H), 3.70 (br d, 2H), | |
| | | 7.10 (d, 3H), 7.30 (dd, 4H), 8.01 (dd, 2H) | |

### Example 522

### 1-(4-Fluorobenzoyl)-4-(2-fluorobenzoyl)piperidine

Magnesium (55 mg, 2.25 mmol) was placed in a flask and covered with ether (6 ml). The reaction was briefly stirred under Argon before the addition of a crystal of iodine. The reaction was cooled to 0° C. before the slow addition of a solution of 2-fluroiodobezene (500 mg, 2.25 mmol) in ether (2 ml). The reaction was then slowly warmed to 30° C. but did not seem to exotherm. At this point 1-(4-fluorobenzoyl)-4-(N-methyl-N-methoxycarbamoyl) piperidine (Method 2; 1 g. 3.38 mmol) was added and the reaction was left to stir for 3 hours. The reaction was then quenched with sat NH₄Cl (10 ml) and extracted with EtOAc (2×10 ml). The combined organic fractions were washed with brine (10 ml) then dried (MgSO₄), filtered and evaporated to yield an oil. Oil purified by column chromatography (10% EtOAc/isohexane to 50% EtOAc/isohexane) to yield an oil. This oil was not clean so the material was further purified by prep LCMS (1-40% over 9.5 mins, MeCN/water, with a constant 5 ml/min 4% formic acid/MeCN) to yield a solid (1 mg, 0.14%). m/z 330.

### Example 523

### 1-(4-Fluorobenzoyl)-4-(pyrid-2-ylcarbonyl)piperidine

Ethyl magnesium bromide (1M soln. in THF—380 μl, 0.3 mmol) was added to a solution of 2-iodopyridine (70 mg, 0.34 mmol) in THF (4 mls) at room temperature under an inert atmosphere. After stirring for 40 minutes, 1-(4-fluorobenzoyl)-4-(N-methyl-N-methoxycarbamoyl)piperidine (Method 2; 120 mg, 0.41 mmol) was added as a solution in THF (1 ml). After stirring at room temperature overnight, more Grignard reagent (1.36 mmol—generated as before) was added. The reaction mixture was stirred for a further 64 h before being quenched with saturated ammonium chloride solution (10 ml). The mixture was extracted with DCM (2×10 ml) before drying (MgSO₄) and the solvent was removed in vacuo. The residue was purified by column chromatography (50% EtOAc/isohexane—80% EtOAc/isohexane). Yield—31 mgs (29%). NMR: 0.95 (m, 2H), 1.77 (m, 2H), 2.00 (m, 2H), 3.14 (m, 2H), 4.17 (m, 1H), 7.08 (m, 2H), 7.45 (m, 3H), 7.85 (m, 1H), 8.06 (m, 1H), 8.68 (m, 1H); m/z 313.

### Example 524

### 1-(4-Fluorobenzoyl)-4-(fur-2-ylcarbonyl)piperidine

n-Butyl lithium (1.6M in hexanes—1.23 ml, 1.97 mmol) was added dropwise under an inert atmosphere to a solution of furan (120 μl, 1.64 mmol) in THF (8 ml) at 0° C. (ice bath). The reaction mixture was allowed to warm to room temperature and stirred for 20 min before re-cooling to 0° C. Magnesium bromide (363 mg, 1.97 mmol) was added to the reaction mixture followed by 1-(4-fluorobenzoyl)-4-(N-methyl-N-methoxycarbamoyl) piperidine (Method 2; 120 mg, 0.4 mmol) in THF (1 ml). The mixture was allowed to warm to room temperature and stirred overnight. The reaction was quenched with saturated ammonium chloride solution (20 ml) and then extracted with EtOAc (2×20 ml). The organic phase was further washed with water (20 ml) before drying (MgSO₄) and solvent removal in vacuo. The resulting yellow gum was triturated with Et₂O/Isohexane to yield a yellow solid (60 mg, 49%). NMR (DMSO-d₆): 1.52 (m, 2H), 1.77 (m, 2H), 3.07 (m, 2H), 3.43 (In, 1H), 6.72 (m, 1H), 7.25 (m, 2H), 7.45 (m, 2H), 7.55 (m, 1H), 7.98 (m, 1H); m/z 302.

### Example 525

### 1-(Fur-2-ylcarbonyl)-4-(3-methoxybenzoyl)piperidine

To a stirred solution of 4-(3-methoxybenzoyl)piperidine (Method 3; 52 mg, 0.24 mmol) and triethylamine (26 mg, 0.26 mmol) in DCM (3 ml) was added 2-furoyl chloride (28 mg, 0.21 mmol). The reaction was stirred at room temperature for 1 hour then worked up. The reaction was transferred to a separating funnel then diluted to ˜10 ml with DCM. The DCM was then washed with 1M HCl (5 ml), sat NaHCO₃ (5 ml) and brine (5 ml) then dried MgSO₄, filtered and evaporated to yield a solid (18 mg, 24%). NMR (DMSO-d₆): 1.60 (m, 2H), 1.90 (m, 2H), 3.25 (t, 2H), 3.75 (m, 1H), 3.90 (s, 3H), 4.30 (d, 2H), 6.60 (m, 1H), 6.90 (m, 1H), 7.20 (m, 1H), 7.50 (m, 2H), 7.60 (d, 1H), 7.75 (s, 1H); m/z 314.

### Example 526

### 1-(4-Fluorobenzoyl)-4-[4-chloro-3-(hydroxymethyl)benzoyl]piperidine

To a stirred solution of 1-(4-fluorobenzoyl)-4-[4-chloro-3-(benzyloxymethyl)benzoyl]piperidine (Example 386; 50 mg, 0.11 mmols) in DCM at −78° C. under Argon was added a 1M solution of BBr₃ in DCM (0.11 ml, 0.11 mmol). The reaction was stirred at −78° C. for 10 minutes then allowed to warm 0° C. and stirred for a further 20 minutes. The reaction was quenched with water (5 ml) and extracted with DCM (2×5 ml). The combined organics were washed with brine (5 ml) then dried (MgSO₄), filtered and evaporated to yield an oil. This oil was purified by column chromatography (10 g Silica, 20 to 60% EtOAc/isohexane) to yield the product as a solid (21 mg, 51%). NMR (DMSd-d₆): 1.60 (m, 2H), 1.90 (m, 2H), 3.20 (m, 2H), 3.70 (m, 1H), 4.00 (br d, 2H), 4.70 (s, 2H), 5.20 (br s, 2H), 7.20 (t, 3H), 7.45 (m, 2H), 7.55 (d, 1H), 7.85 (m, 1H), 8.15 (m, 1H); m/z 376.

### Example 527

### 1-(t-Butoxycarbonyl)-4-[4-(6-bromonaphth -2-ylthio)benzoyl]piperidine

60% Sodium hydride (717 mg, 18 mmol) was suspended in anhydrous dimethylformamide (50 ml) under nitrogen at 5° C. To this was added portion-wise 6-bromo naphthalene-2-thiol (3.89 g, 16 mmol). The mixture was stirred at 5° C. for 30 minutes. 1-(t-Butoxycarbonyl)-4-(4-fluorobenzoyl)piperidine (Reference Example 12; 5.00 g 16 mmol) was then added to the solution and the reaction heated at 60° C. for 16 hours. The solution was poured into water (75 ml) and washed with EtOAc (2×75 ml). The organic phases were combined then washed with water then brine. The solution was dried over MgSO₄, after filtration and evaporation a solid was isolated. This was recrystallised from EtOAc/isohexane resulting in a cream solid (2.96 g, 35%). NMR (DMSO-d₆) 1.37 (s, 11H), 1.72 (m, 2H), 2.86 (m, 2H), 3.52 (m, 1H), 3.92 (m, 2H), 7.31 (d, 2H), 7.55 (d, 1H), 7.69 (d, 1H), 7.93 (m, 4H), 8.17 (s, 1H), 8.26 (s, 1H); m/z 470.

### Example 528

### 1-(4-Fluorobenzoyl)-4-(thiazol-2-ylcarbonyl)piperidine

n-Butyl lithium (1.6M in hexanes—275 μl, 0.44 mmol) was added dropwise under an inert atmosphere to a solution of thiazole (54.5 mg, 0.4 mmol) in THP (2 ml) at −78° C. The reaction mixture was stirred at −78° C. for 10 min before 1-(4-fluorobenzoyl)-4-(N-methyl-N-methoxycarbamoyl)piperidine (Method 2; 118 mg, 0.4 mmol) in THF (2 ml) was added. The mixture was stirred at −78° C. for 30 min before being allowed to warm to room temperature and stirred overnight. The reaction was quenched with saturated ammonium chloride solution (8 ml) and then extracted with DCM (8 ml). The biphasic mixture was passed through a phase separation cartridge and the solvent was removed in vacuo. The resulting residue was purified by chromatography (EtOAc/Isohexane gradient) to yield the product. (15 mg, 12%). NMR: 1.2-2.2 (m, 6H), 3.10 (m, 2H), 3.90 (m, 1H), 7.12 (m, 2H), 7.43 (m, 2H), 7.71 (d, 1H), 8.03 (d, 1H); m/z 319.

### Examples 529-534

The procedure described in Example 528 was repeated using the appropriate heterocycle to replace thiazole to give the compounds shown below.

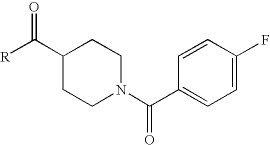

| Ex | R¹ | NMR | M/z |
|---|---|---|---|
| 529 | 4,5-Dimethylthiazol-2-yl | | 347 |
| 530 | Benzothiazol-2-yI | | 369 |
| 531 | 5-Chlorobenzofuran-2-yl | 1.90 (m, 6H), 3.17 (m, 2H), 3.50 (m, 1H), 7.12 | 386 |
| | | (m, 2H), 7.48 (m, 5H), 7.70 (d, 1H) | |
| 532 | Benzofuran-2-yl | | 352 |
| 533 | 5-Chlorobenzothien-2-yl | 1.07 (m, 2H), 1.56 (m, 2H), 1.92 (m, 2H), 3.15 | 402 |
| | | (m, 2H), 3.48 (m, 1H), 7.15 (m, 3H), 7.25 (m, | |
| | | 1H), 7.44 (m, 2H), 7.81 (d, 1H), 7.91 (dd, 1H) | |
| 534 | Benzothien-2-yl | 1.95 (m, 6H), 3.17 (m, 2H), 3.55 (m, 1H), 7.11 | 368 |
| | | (m, 2H), 7.44 (m, 4H), 7.88 (m, 2H), 8.02 (s, 1H) | |

### Example 535

### 1-(4-Fluorobenzoyl)-4-(5-cyanofur-2-ylcarbonyl)

The procedure described in Example 528 was repeated using 2-furonitrile instead of thiazole and lithium diisopropylamide (2M in THF/heptane) instead of n-butyl lithium. The product was isolated as a brown gum. NMR (DMSO-d6): 1.50 (m, 2H), 1.82 (m, 2H), 3.07 (m, 4H), 3.48 (m, 1H), 7.24 (m, 2H), 7.43 (m, 2M), 7.71 (d, 1H), 7.76 (d, 1H); m/z 327.

### Reference Example 11

### 1-Benzyl4-benzoylpiperidine

1,2-Dibromoethane (19 μl, 0.22 mmol) and a crystal of iodine were added to magnesium turnings (97 mg, 4 mmol) under an inert atmosphere. 1-Benzyl-4-bromopiperidine (1 g, 4 mmol) was added slowly as a solution in THF (8 ml). Upon complete addition, the reaction mixture was heated at reflux for 10 minutes before cooling to room temperature. Benzonitrile (360 μl, 3.5 mmol) was added as a solution in THF (4 ml) and the reaction mixture heated at reflux for 3 hours. After cooling, saturated ammonium chloride solution (15 ml) was added, followed by EtOAc (15 ml). The organic phase was further washed with water (15 ml) and then dried over magnesium sulphate. The solvent was removed under reduced pressure and the residue purified by chromatography (eluent: DCM/methanol/NH3—20/0.5/0.05) to yield the product as a brown gum (399 mg, 41%). NMR (DMSO-d6): 1.60 (m, 2H), 1.75 (m, 2H), 2.100 (m, 2H), 2.84 (m, 2H), 3.37 (m, 1H), 3.48 (s, 21), 7.27 (m, 5H), 7.50 (m, 2H), 7.61 (m, 1H), 7.94 (d, 2H); m/z 280.

### Example 536

### 1-Cyclopropylcarbonyl-4-(5-methylthien-2-yl)piperidine

1,2-Dibromoethane (35 μl, 0.4 mmol) and a crystal of iodine were added to magnesium turnings (228 mg, 4 mmol) under an inert atmosphere. 1-Benzyl4-bromopiperidine (2 g, 7.87 mmol) was added slowly as a solution in THF (10 ml). Upon complete addition, the reaction mixture was heated at reflux for 10 minutes before cooling to 0° C. 5-Methyl-2-thiophenecarboxaldehyde (15.74 mmol) was added as a solution in THF (5 ml) and the reaction mixture was warmed to room temperature and stirred for 16 hours. Saturated ammonium chloride solution (20 ml) was added, followed by EtOAc (20 ml). The organic phase was further washed with water (20 ml) and then dried over magnesium sulphate. The solvent was removed under reduced pressure and the residual gum was dissolved in DCM (15 ml) and stirred under argon. α-Chloroethyl chloroformate (826 μl, 8 mmol) was added to the solution and stirred at room temperature for 30 min before concentrating in vacuo. The resulting residue was dissolved in methanol (10 ml) and the solution heated at reflux for 40 min before solvent removal. The product obtained was taken up in DCM (20 ml), triethylamine (2.19 ml, 15.74 mmol) was added and the solution was split into 5 parts. One part of the solution (1.574 mmol) was stirred under an inert atmosphere and cyclopropanecarbonyl chloride (1.574 mmol) was added. The reaction mixture was stirred for 64 hours before quenching with saturated ammonium chloride solution (8 ml) and addition of DCM (8 ml). The biphasic mixture was passed through a phase separation cartridge and the solvent was removed in vacuo. The resulting residue was purified by chromatography (20% EtOAc/isohexane to 100% EtOAc gradient) to yield the product (49 mg, 11%). NMR: 0.76 (m, 2H), 1.00 (m, 2H), 1.62 (m, 2H), 1.78 (m, 2H), 1.93 (m, 2H), 2.57 (s, 3H), 3.30 (in, 2H), 4.30 (m, 1H), 4.58 (m, 1H), 6.82 (d, 1H), 7.58 (d, 1H); m/z 278.

### Example 537-550

The procedure described in Example 536 was repeated using the appropriate reagents to replace ‘5-Methyl-2-thiophenecarboxaldehyde’ and ‘cyclopropanecarbonyl chloride’ to give the compounds shown below.

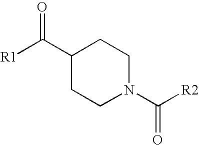

| Ex | R1 | R2 | M/z |
|---|---|---|---|
| 537 | 5-methylthien-2-yl | 4-Trifluoromethoxyphenyl | 398 |
| 538 | 3-Trifluorophenyl | 4-Cyanophenyl | 387 |
| 539 | 3-Trifluorophenyl | 4-Trifluoromethoxyphenyl | 446 |
| 540 | 3-Trifluorophenyl | 4-Fluorophenyl | 380 |
| 541 | 3-Trifluorophenyl | Cyclopropyl | 326 |
| 542¹ | 3-Trifluorophenyl | Pyridin-2-yl | 363 |
| 543² | Thien-3-yl | 4-Trifluoromethoxyphenyl | 384 |
| 544 | Thien-3-yl | 4-Fluorophenyl | 318 |
| 545 | 4-Chlorothien-2-yl | 4-Fluorophenyl | 352 |
| 546 | 4-Chlorothien-2-yl | 4-Difluoromethoxyphenyl | 400 |
| 547 | 4-Chlorothien-2-yl | Quinolin-2-yl | 385 |
| 548 | 4,5-Dimethylfur-2-yl | 4-Fluorophenyl | 330 |
| 549 | 4,5-Dimethylfur-2-yl | Cyclohexyl | 318 |
| 550 | 5-(Thien-2-yl)thien-2-yl | 4-Difluoromethoxyphenyl | 448 |

¹Method used corresponding carboxylic acid and 1-(3-dimethylaminopropyl)-3-ethylcarbodiimide hydrochloride instead of corresponding acid chloride.
²NMR: 1.60-2.00 (m, 6H), 3.12 (m, 2H), 3.37 (m, 1H), 7.28 (m, 2H), 7.38 (m, 1H), 7.49 (m, 2H), 7.59 (m, 1H), 8.09 (m, 1H).

### Reference Example 12

### 1-(t-Butoxycarbonyl)-4-(4-fluorobenzoyl)piperidine

4-(4-Fluorobenzoyl)piperidine p-toluenesulfonate (20.00 g, 53 mmol) was dissolved in (200 ml) and triethylamine (14.68 ml, 106 mmol). To this was added dropwise a solution tert-butyl dicarbonate (12.65 g, 58 mmol) in DCM (100 ml). The mixture was stirred at ambient temperature for 3 hours. The solution was then washed with water (100 ml) then ted NaHCO₃. The solution was then dried over MgSO₄, after filtration and evaporation an oil was isolated. This was chromatographed on silica eluting with 0-20% EtOAc/isohexane. The relevant fractions were combined to afford a white solid (14.22 g, 88%). NMR (DMSO-d₆) 1.38 (s, 11H), 1.72 (m, 2H), 2.89 (m, 2H), 3.60 (m, 1H), 3.95 (m, 2H), 7.32 (t, 2H), 8.05 (m, 2H); m/z 308.

### Example 551

### 1-(Cyclopentylcarbonyl)-4-(4-chorobenzoyl)-4-ethylpiperidine

The title compound was prepared using the same procedure as was used for Examples 130-345 and Reference Examples 3-5 above. The method type was “XXe”. M/z 364.4.

### Example 552

### 1-(4-Fluorobenzoyl)-4-(3-cyanobenzoyl)piperidine

1-(4-Fluorobenzoyl)-4-etboxycarbonyl-4-(3-cyanobenzoyl)piperidine (Method 13) was split into two portions of 0.19 mmol and heated with lithium chloride (0.37 mmol) and water (several drops) in dimethyl acetamide (2 ml) in the microwave at 200° C. for 10-15 minutes. The reaction mixture was concentrated in vacuo, the residue partitioned between water and DCM and passed through a phase separation cartridge, the crude material was purified on a Biotage Quad3+ flash chromatography system eluting with 25% EtOAc/isohexane to furnish the title compound. NMR: 8.21 (1H, s), 8.19 (1H, d), 7.87 (1H, d), 7.65 (1H, dd), 7.43 (2H, dd), 7.12 (2H1 dd), 3.53 (1H, m), 3.19 (2H, bs), 2.0-1.71 (4H, m), 1.30 (1H, m); m/z 332.5.

### Example 553

### 1-(2-Methyl-4,5,6,7-tetrahydrobenzofuran-3-ylcarbonyl)-4-(4-fluorobenzoyl)piperidine

The title compound was prepared using the same procedure as was used for Examples 130-345 and Reference Examples 3-5 above. The method type was “YYb”. M/z 370.

### Example 554

### 1-(Pyrrolidin-1-ylcarbonyl)-4-(4-fluorobenzoyl)piperidine

To a solution of pyrrolidine (81 μl, 1.0 mmol) and DIEA (174 μl, 1.0 mmol) in DCM (5 ml) was added a pre-prepared solution of 4-(4-fluorobenzoylpiperidine)hydrochloride (293 mg, 1.2 mmol) and triphosgene (297 mg, 1.0 mmol) in DCM (5 ml). Following completion of the addition DIEA (2.0 mmol) was added to the reaction mixture and stirred for 16 hours at room temperature. After this time, further triphosgene (1.0 mmol), pyrrolidine (1.0 mmol) and DIEA (1.0 mmol) were added to the reaction mixture to encourage reaction to completion. After stirring at room temperature for a further 24 hours the reaction had reached completion and was worked up. Reaction mixture was transferred to a separating funnel and diluted to approximately 5 ml with DCM. The DCM was washed with 2M HCl (10 ml), water (10 ml) and brine (5 ml) then dried (MgSO₄), filtered and evaporated to yield the crude product as a yellow oil. Purification by prep LCMS yielded the product as a yellow solid (85 mg, 0.28 mmol, 28%). NMR (DMSO-d₆): 1.48 (q, 2H), 1.71 (br s, 6H), 2.84 (t, 2H), 3.23 (t, 5H), 3.55 (dt, 1H), 3.63 (br d, 2H), 7.34 (t, 2H), 8.06 (dd, 2H); m/z 305.

### Example 555

### 1-(t-Butoxycarbonyl)-4-[4-(6-bromonaphth-2-ylsulphonylbenzoyl]piperidine

1-(t-Butoxycarbonyl)-4-[4-(6-bromonaphth-2-ylthio)benzoyl]piperidine (Example 527; 2.93 g, 5.6 mmol) was dissolved in DCM (50 ml), to this was added 3-chloroperoxybenzoic acid (5.79 g, 17 mmol). The reaction was stirred for 18 hours before washing with 2M NaOH (25 ml), drying (MgSO₄) before evaporation to give crude material. The compound was purified by chromatography on silica gel eluting with 0-10% EtOAc in toluene. The title compound was obtained as a white solid (958 mg, 31%). NMR (DMSO-d₆) 1.31 (m, 11H), 1.71 (m, 2H), 2.86 (m, 2H), 3.59 (m, 1H), 3.89 (m, 2H), 7.83 (d, 1H), 7.97 (d, 1H), 8.14 (m, 6H), 8.34 (s, 1H), 8.79 (s, 1H); m/z 559.

### Example 556

### 4-[4-(6-Bromonaphth-2-ylsulphonyl)benzoyl]piperidine hydrochloride

1-(t-Butoxycarbonyl)-4-[4-(6-bromonaphth-2-ylsulphonyl)benzoyl]piperidine (Example 555; 944 mg, 1.7 mmol) was dissolved in EtOAc (25 ml) then treated with 4M HCl in EtOAc then stirred for 3 hours. The slurry was then evaporated then slurried in ether (40 ml) before filtration to give the title compound as a white solid (744 mg, 89%). NMR (DMSO-d₆) 1.80 (m, 4H), 2.97 (m, 2H), 3.26 (m, 2H), 3.74(m, 1H), 7.83 (d, 1H), 7.97 (d, 1H), 8.14 (m, 6H), 8.34 (s, 1H), 8.79 (m, 2H), 9.04 (bs, 1H); m/z 458.

### Example 557

### 1-[2-(t-Butoxycarbonylamino)acetyl]-4-[4-(6-bromonaphth-2-ylsulphonyl)benzoyl]piperidine

4-[4-(6-Bromonaphth-2-ylsulphonyl)benzoyl]piperidine hydrochloride (Example 556; 200 mg, 0.41 mmol) was added to a solution of N-(tert-butoxycarbonyl)glycine (78 mg, 0.45 mmol), 1-hydroxybenzotriazole monohydrate (68 mg, 0.45 mmol), 1-ethyl-3-(3- dimethylaminopropyl)carbodiimide hydrochloride (86 mg, 0.45 mmol) and 4-methylmorpholine (0.093 ml, 0.85 mmol) in N,N-dimethylformamide (20 ml). The mixture was stirred at ambient temperature for 16 hours. The volatiles were removed by evaporation and the residue was dissolved in DCM (20 ml) and water (10 ml), the layers were separated before washing with 2M HCl then saturated NaHCO₃. Evaporation afforded a white solid. The compound was purified by chromatography on silica gel eluting with 0-2% methanol in DCM. The title compound was obtained as a white solid (198 mg, 80%). NMR (DMSO-d₆) 1.40 (m, 11H), 1.77 (m, 2H), 2.74 (m, 2H), 3.11 (m, 1H), 3.71 (m, 4H), 4.27 (m, 1H), 6.66 (m, 1H), 7.83 (d, 1H), 7.97 (d, 1H), 8.14 (m, 6H), 8.34 (s, 1H), 8.79 (s, 1H); m/z 615.

### Example 558

### 1-(2-Aminoacetyl)-4-[4-(6-bromonaphth-2-ylsulphonyl)benzoyl]piperidine hydrochloride

The title compound was prepared from 1-[2-(t-butoxycarbonylamino)acetyl]-4-[4-(6-bromonaphth-2-ylsulphonyl)benzoyl]piperidine (Example 557) by a the procedure of Example 556. NMR (DMSO-d₆) 1.43 (m, 2H), 1.80 (m, 2H), 2.84 (m, 1H), 3.17 (m, 1H), 3.80 (m, 4H), 4.31 (m, 1H), 7.83 (d, 1H), 7.97 (d, 1H), 8.14 (m, 6H), 8.34 (s, 1H), 8.79 (s, 1H); m/z 515.

### Example 559

### 1-(Imino(phenol)methyl)-4-[4-(6-bromonaphth-2-ylsulphonyl)benzoyl]piperidine dihydrochloride

4-[4-(6-Bromonaphth-2-ylsulphonyl)benzoyl]piperidine hydrochloride (Example 556; 150 mg, 0.30 mmol), methyl benzimidate hydrochloride (104 mg, 0.61 mmol) and triethylamine (0.17 ml, 1.2 mmol) were dissolved in methanol/chloroform (20 ml) and stirred for 16 hours. Methyl benzimidate hydrochloride (104 mg, 0.61 mmol) and triethylamine (0.17 ml, 1.2 mmol) were further added followed by stirring for 16 hours. The solvent was evaporated before the compound was purified by chromatography on silica gel eluting with 0-15% ethanol in DCM. The compound was purified further on a reverse phase bond elute. The title compound was obtained as a white solid (90 mg, 47%). NMR, DMSO-₆ 1.80 (m, 4H), 3.33 (m, 4H), 3.84 (m, 1H), 7.61 (m, 5H), 7.83 (d, 1H), 7.97 (d, 1H), 8.14 (m, 6H), 8.34 (s, 1H), 8.79 (s, 1H); m/z 561.

Preparation of Starting Materials

The starting materials for the examples above are either commercially available or are readily prepared by standard methods from known materials. For example, the following reactions are an illustration, but not a limitation, of some of the starting materials used in the above reactions.

Method

### 1-(4-Fluorobenzoyl)-4-(ethoxycarbonyl)piperidine

To a stirred solution of ethylisonipecotate (2.5 g, 0.016 mol) and triethylamine (1.77 g, 0.017 mol) in DCM (100 ml) was added 4-flurobenzoyl chloride (2.39 g, 0.015 mol). The reaction was stirred at room temperature for one hour then worked up. The reaction was transferred to a separating funnel and diluted to ˜150 ml with DCM. The DCM was washed with 1M HCl (100 ml), sat NaHCO₃ (100 ml) and brine (50 ml) then dried (MgSO₄), filtered and evaporated to yield an oil (3.67 g, 83%). NMR (DMSO-d₆): 1.20 (t, 3H), 1.60 (m, 2H), 1.90 (m, 2H), 2.65 (m, 1H), 3.10 (m, 2H), 3.95 (br d, 2H), 4.10 (q, 2H), 7.25 (t, 2H), 7.55 (m, 2H); m/z 280.

Method 2

### 1-(4-Fluorobenzoyl)-4-(N-methyl-N-methoxycarbamoyl)piperidine

To a stirred solution of 1-(4-fluorobenzoyl)-4-(ethoxycarbonyl)piperidine (Method 1; 1 g, 3.58 mmol) in anhydrous THF (30 ml) was added N,O-dimethylhydroxylamine hydrochloride (350 mg, 3.58 mmol). The resulting solution was cooled to −10° C. before the addition of a 2M solution of isopropyl magnesium chloride (3.58 ml, 7.16 mmol). The reaction was stirred at −10° C. for 15 minutes then allowed to warm to room temperature. The reaction was stirred at room temperature for 60 minutes before the addition of further isopropyl magnesium chloride (0.18 ml, 0.36 mmol). The reaction was then stirred for a further 10 minutes before working up. The reaction was quenched with sat NH₄Cl solution (˜20 ml) then extracted with EtOAc (2×20 ml). The combined organic layers were washed with brine then dried (MgSO₄), filtered and evaporated to yield the title compound (880 mg, 84%). NMR (DMSO-d₆): 1.60 (m, 2H), 1.80 (m, 2H), 3.00 (m, 1H), 3.10 (m, 2H), 3.15 (s, 3H), 3.70 (s, 3H), 4.05 (m, 2H), 7.20 (t, 2H), 7.45 (m, 2H); m/z 295.

Method 3

### 4-(3-Methoxybenzoyl)piperidine

To a stirred 1M solution of 3-methoxyphenyl magnesium bromide in THF (12 ml, 0.012 mols) was added a solution of 1-acetylpiperidine-4-carbonitrile (1 g, 6.57 mols) in THF (4 ml). The reaction was then left to stir overnight in the dark. The reaction was quenched with sat NH₄Cl and then warmed to 40° C. and stirred at this temperature for 1 hour. The volatile organics were removed under reduced pressure and the resulting aqueous layer was extracted with ether (2×20 ml). The organic layers were combined, washed with brine then evaporated to-yield an oil. This oil was dissolved in dioxane (7 ml) and treated with 5M HCl (7 ml). The reaction was heated to 100° and stirred at this temperature-overnight. The reaction was the cooled to room temperature and evaporated under reduced pressure. The resulting crude material was dissolved in DCM and washed with 2M NaOH, water and brine. The solvent was evaporated under reduced pressure to yield a yellow oil. This oil was dissolved in a small amount of MeOH and loaded onto an SCX-2 column. The column was eluted with MeOH until no further impurities eluted off. The desired product was then eluted with 1% NH₃/MeOH to yield an oil (52 mg, 4%). m/z 220.

Method 4

### 3-Methyl-4-(4-fluorobenzoyl)piperidine hydrochloride

To a stirred solution of 1-(t-butoxycarbonyl)-3-methyl-4-(N-methyl-N-methoxycarbamoyl)piperidine (Method 5; 85 mg, 0.3 mmol) in anhydrous THP (2 ml) at 0° C. was added a 1M solution of 4-fluorophenyl magnesium bromide in THF (1 ml, 1 mmol). The reaction was stirred at 0° C. for 1 hour then allowed to warm to room temperature and stirred for a further 90 minutes. At this stage further 4-fluorophenyl magnesium bromide (0.5 ml, 0.5 mmol) was added and the reaction was stirred for a further hour. The reaction was quenched with sat NH₄Cl solution (˜5 ml) then extracted with EtOAc (2×5 ml). The combined organic layers were then washed with brine (˜5 ml), dried (MgSO₄), filtered and evaporated to yield an oil. This oil was dissolved in DCM (˜1 ml) and treated with TFA (˜0.1 ml) then left to stir overnight at room temperature. The reaction mixture was then transferred to a separating funnel and diluted to ˜5 ml with DCM. The DCM layer was then washed with 1M NaOH and evaporated to yield an oil. This oil was eluted through an Isolute SCX-2 column using MeOH. When all impurities had eluted off the product was eluted with 1% NH₃/MeOH. This product was dissolved in ether then treated with 1.1 eq of 1M HCl in ether. The resulting suspension was evaporated under reduced pressure lo yield a solid. This solid was left under high vac overnight to yield the product as the hydrochloride salt (22 mg, 30%). NMR (DMSO-d₆): 0.90 (d, 3H), 1.90 (m, 1H), 2.00 (m, 2H), 2.40 (m, 1H), 3.20 (m, 3H), 3.90 (m, 1H), 7.30 (t, 2H), 8.05 (m, 21), 8.60 (br s, 2H); m/z 222.

Method 5

### 1-(t-Butoxycarbonyl)-3-methyl-4-(N-methyl-N-methoxycarbamoyl)piperidine

To a stirred solution of N-Boc-3-methyl-4-piperidine carboxylic acid (100 mg, 0.41 mmol), N,O-dimethyl hydroxylamine hydrochloride (40 mg, 0.41 mmol) and N-methyl morpholine (41 mg, 0.41 mmol) in DCM (5 ml) was added 1-ethyl-3-(3-dimethylaminopropyl)carbodiimide hydrochloride (79 mg, 0.41 mmol). The resulting solution was stirred at room temperature for 48 hours. The reaction mixture was transferred to a separating funnel and washed with 1M HCl (2×5 ml), sat NaHCO₃ (5 ml) and brine (5 ml) then dried (MgSO₄), filtered and evaporated to yield a solid (85 mg, 73%). NMR (DMSO-d₆): 0.85 (d, 3H), 1.45 (s, 9H), 1.47 (m, 1H), 1.80 (m, 1H), 2.10 (m, 1H), 3.05 (m, 3H), 3.10 (s, 3H), 3.20 (m, 1H), 3.65 (m, 1H), 3.70 (s, 3H), 3.80 (m, 1H).

Method 6

### 1-(4-Fluorophenylsulphonyl)-4-(ethoxycarbonyl)piperidine

To a stirred solution of ethylisonipecotate (15 g, 0.095 mol) and triethylamine (10.6 g, 0.105 mol) in DCM (380 ml) at 0° C. was added a solution of 4-fluorobenzenesulfonylchloride (17.6 g, 0.09 mol) in DCM (20 ml). The reaction was stirred at 0° C. for 10 minutes then allowed to warm to room temperature and stirred for a further 2 hours. The reaction mixture was transferred to a separating funnel and washed with 2M HCl (80 ml), water (40 ml), sat NaHCO₃ (40 ml) and brine (40 ml) and then dried (MgSO₄), filtered and evaporated to yield a white solid (25.75 g, 88%). NMR (DMSO-d₆): 1.15 (t, 3H), 1.55 (m, 2H), 1.85 (m, 2M), 2.35 (m, 1H), 2.45 (m, 2H), 3.50 (m, 2H), 4.05 (q, 2H), 7.45 (t, 2H), 7.80 (m, 2H); m/z 316.

Method 7

### 1-(Isopropylsulphonyl)-4-(ethoxycarbonyl)piperidine

The title compound was prepared by the procedure of Method 6. NMR (DMSO₆): 1.20 (m, 9H), 1.50 (m, 2H), 1.85 (m, 2H), 2.55 (m, 1H), 2.85 (m, 2H), 3.30 (m, 1H), 3.60 (m, 2H), 4.10 (q, 2H); m/z 264.

Method 8

### 1-(4-Fluorophenylsulphonyl)-4-(N-methyl-N-methoxycarbamoyl)piperidine

To a stirred solution of 1-(4-fluorophenylsulphonyl)-4-(ethoxycarbonyl)piperidine Method 6; 8 g, 0.025 mol) and N,O-dimethyl hydroxylamine hydrochloride (2.49 g, 0.025 mol) in anhydrous THF (200 ml) at 0° C. was added a 2M solution of isopropyl magnesium chloride in THF (26 ml, 0.053 mol). The reaction was stirred at 0° C. for ten minutes then allowed to warm to room temperature and left to stir for two and a half hours. The reaction was quenched with sat NH₄Cl solution (100 ml) and extracted with EtOAc (2×100 ml). The combined organic phases were washed with brine then dried (MgSO₄), filtered and evaporated to yield an oil. This oil was purified by column chromatography (50 g Silica, 20% EtOAc/isohexane to 60% EtOAc/isohexane) to yield an oil which crystallised on standing (6 g, 73%). NMR (DMSO-d₆): 1.60 (m, 2H), 1.80 (m, 2H), 2.55 (m, 2H), 2.70 (m, 1H), 3.05 (s, 3H), 3.65 (m, 5H), 7.40 (t, 2H), 7.80 (m, 2H); m/z 331.

Method 9

### 1-(Isopropylsulphonyl)-4-(N-methyl-N-methoxycarbamoyl)piperidine

The title compound was prepared by the procedure of Method 8, except the product did not require chromatography. NMR (DMSO-d₆): 1.20 (d, 6H), 1.50. (m, 2H), 1.75 (m, 2H), 2.85 (m, 1H), 2.95 (m, 2H), 3.10 (s, 3H), 3.30 (m, 1H), 3.70 (s, 3H); m/z 279.

Method 10

### 5-Bromo-2-chloro-1-(benzyloxymethyl)phenyl

To a stirred solution of 5-bromo-2-chloro benzyl alcohol (2.5 g, 0.011 mols) in DMF (100 ml) was added NaH (60% suspension) (497 mg, 0.012 mols). The resulting reaction was stirred at room temperature for 30 minutes before the addition of benzyl bromide (1.79 g, 0.01 mols). The reaction was stirred at room temperature for 3 hours then quenched with sat NH₄Cl solution (10 ml). The volatiles were removed under reduced pressure and the resulting slurry was partitioned between EtOAc and water (˜100 ml of each). The layers were separated and the aqueous was re-extracted with EtOAc (˜30 ml). The organic layers were combined, washed with brine (30 ml) then dried (MgSO₄), filtered and evaporated to yield an oil. This oil was purified by column chromatography (20 g Silica, isohexane to 10% EtOAc/isohexane) to yield the product as an oil (1.32 g, 42%). NMR (DMSO-d₆): 4.58 (s, 2H), 4.60 (s, 2H), 7.30 (m, 1H), 7.35 (m, 4H), 7.40 (s, 1H), 7.50 (m, 1H), 7.65 (m, 1H); m/z 310.

Method 11

### 5-Bromo-2-chloro-1-(methoxymethyl)phenyl

To a stirred solution of 5-Bromo-2-Chloro-benzyl alcohol (5.46 g, 0.025 mols) in anhydrous THF (50 ml) was added NaH (60% suspension) (1.18 g, 0.03 mols). The resultant reaction was stirred at room temperature for 20 minutes before the addition of methyl iodide (4.68 g, 0.033 mols). The reaction was left to stir for 3 hours then quenched with 2M HCl (˜20 ml) and extracted with EtOAc (2×15 ml). The combined organic layers were washed with brine (20 ml) then dried (MgSO₄), filtered and evaporated to yield an oil. This oil was purified by column chromatography (50 g Silica, 20% EtOAc/isohexane) to yield a colourless oil (5.46 g, 93%). NMR (DMSO-d₆): 3.35 (s, 3H), 4.45 (s, 2H), 7.40 (d, 1H), 7.50 (m, 1H), 1.60 (m, 1H); m/z: 234.

Method 12

### 1-(4-Fluorobenzoyl)-4-ethoxycarbonylpiperidine

To a solution of ethyl isonipecotate (95 mmol) and triethylamine (114 mmol) in DCM (350 ml) at 5° C. was added 4-fluorobenzoyl chloride (90 mmol). The resultant suspension was allowed to stir at this temperature for 3 hours. The reaction mixture was then washed with 1M HCl, saturated NaHCO₃ and brine, dried over MgSO₄ and the filtrate concentrated in vacuo to afford the title compound. M/z: 280.5.

Method 13

### 1-(4-Fluorobenzoyl )-4-ethoxycarbonyl-4-(3-cyanobenzoyl)piperidine

A solution of 1-(4-fluorobenzoyl)-4-ethoxycarbonylpiperidine (Method 12; 1.2 mmol) in THF (10 ml) was added to LHMDS (3 mmol) at room temperature and under argon, 3-cyanobenzoyl chloride (4.8 mmol) was then added and the reaction allowed to stir at room temperature over night. The reaction mixture was quenched with water, concentrated in vacuo, and the residue partitioned between water and DCM before being passed through a phase separation cartridge. The crude product was purified on a Biotage Quad3+ flash chromatography system, eluting with 25% EtOAclisohexane to give the title compound. M/z: 409.2.

1. A method for inhibiting 11βHSD1. comprising administering to a warm-blooded animal a compound of formula (I): 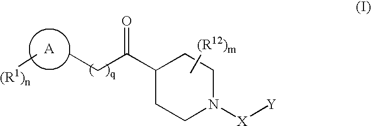
wherein:
Ring A is selected from carbocyclyl or heterocyclyl; wherein if said heterocyclyl contains an —NH— moiety that nitrogen may be optionally substituted by R⁹;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more R³; and wherein if said heterocyclyl contains an —NH— moiety that nitrogen may be optionally substituted by R⁴;
n is 0-5; wherein the values of R¹ may be the same or different;
X is a direct bond, —C(O)—, —S(O)₂—, —C(O)NR¹¹—, —C(S)NR¹¹—, —C(O)O—, —C(═NR¹¹)— or —CH₂—; wherein R¹¹ is selected from hydrogen, C₁₋₄alkyl, carbocyclyl and heterocyclyl;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more R⁶; and wherein if said heterocyclyl contains an —NH— moiety that nitrogen may be optionally substituted by R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸; and wherein if said heterocyclyl contains an —NH— moiety that nitrogen may be optionally substituted by R¹³;
R⁴, R⁵, R⁷ R⁹ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰OC(O)—, —OC(O)NR¹⁰— or —SO₂NR¹⁰—; wherein a is 0 to 2; wherein R¹⁰ is selected from hydrogen and C₁₋₄alkyl;
R¹² is hydroxy, methyl, ethyl or propyl;
m is 0 or 1;
q is 0 or 1;
or a pharmaceutically acceptable salt thereof.

2. The method of claim 1, wherein Ring A is phenyl, 1,3-benzodioxolyl, thienyl, cyclopentyl, pyridyl, furyl, thiazolyl, 1,3-benzothiazolyl, benzofuryl or benzothienyl; or a pharmaceutically acceptable salt thereof.

3. The method of claim 1, wherein
R¹ is a substituent on carbon and is selected from halo, cyano, C₁₋₄alkyl, C₁₋₄alkoxy, N,N—(C₁₋₄alkyl)₂amino, C₁₋₄alkylS(O)ₐ wherein a is 0 to 2, carbocyclyl and carbocyclylC₀₋₄alkylene-Z-; wherein R¹ may be optionally substituted on carbon by one or more R³; wherein R³ is selected from halo, hydroxy, C₁₋₄alkoxy, heterocyclyl and carbocyclylC₀₋₄alkylene-Z-; and
Z is —S(O)ₐ— or —O—; wherein a is 0 to 2;
or a pharmaceutically acceptable salt thereof.

4. The method of claim 1, wherein n is 0-3; and wherein the values of R¹ may be the same or different; or a pharmaceutically acceptable salt thereof.

5. The method of claim 1, wherein X is —C(O)—; or a pharmaceutically acceptable salt thereof.

6. The method of claim 1, wherein
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by R⁵; wherein R² is a substituent on carbon and is selected from halo, nitro, cyano, amino, trifluoromethyl, trifluoromethoxy, C₁₋₄alkyl, C₁₋₄alkoxy, C₁₋₄alkanoyl, N—(C₁₋₄alkyl)amino, N,N—(C₁₋₄alkyl)₂amino, C₁₋₄alkanoylamino, C₁₋₄alkylS(O)ₐ wherein a is 0 to 2, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more R⁶;
R⁶ is selected from halo, nitro, cyano, trifluoromethyl, C₁₋₄alkyl, C₂₋₄alkenyl, C₁₋₄alkoxy, N,N—(C₁₋₄alkyl)₂amino, C₁₋₄alkylS(O)ₐ wherein a is 0 to 2, C₁₋₄alkoxycarbonylamino, carbocyclyl, heterocyclyl and carbocyclylC₀₋₄alkylene-Z-; wherein R⁶ may be optionally substituted on carbon by one or more R⁸;
R⁵ is selected from C₁₋₄alkyl, C₁₋₄alkanoyl and C₁₋₄alkoxycarbonyl;
Z is —S(O)ₐ—, —O—, —NR¹⁰—, —C(O)— or —OC(O)NR¹⁰—; wherein a is 0 to 2; wherein R¹⁰ is selected from hydrogen; and
R⁸ is selected from halo;
or a pharmaceutically acceptable salt thereof.

7. The method of claim 1 wherein R¹² is 4-methyl, 4-ethyl, 4-propyl or 3-methyl; or a pharmaceutically acceptable salt thereof.

8. The method of claim 1, wherein q is 0; or a pharmaceutically acceptable salt thereof.

9. The method of claim 1, wherein:
Ring A is phenyl, 1,3-benzodioxolyl, thienyl, cyclopentyl, pyridyl, furyl, thiazolyl, 1,3-benzothiazolyl, benzofuryl or benzothienyl;
R¹ is a substituent on carbon and is selected from halo, cyano, C₁₋₄alkyl, C₁₋₄alkoxy, N,N—(C₁₋₄alkyl)₂amino, C₁₋₄alkylS(O)ₐ wherein a is 0 to 2, carbocyclyl and carbocyclylC₀₋₄alkylene-Z-; wherein R¹ may be optionally substituted on carbon by one or more R³; wherein
R³ is selected from halo, hydroxy, C₁₋₄alkoxy, heterocyclyl and carbocyclylC₀₋₄alkylene-Z-; and
Z is —S(O)ₐ— or —O—; wherein a is 0 to 2;
X is a direct bond, —C(O)—, —S(O)₂—, —C(O)NR¹¹—, —C(S)NR¹¹—, —C(O)O—, —C(═NR¹¹)— or —CH₂—; wherein R¹¹ is selected from hydrogen, C₁₋₄alkyl, carbocyclyl and heterocyclyl;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by R⁵; wherein R² is a substituent on carbon and is selected from halo, nitro, cyano, amino, trifluoromethyl, trifluoromethoxy, C₁₋₄alkyl, C₁₋₄alkoxy, C₁₋₄alkanoyl, N—(C₁₋₄alkyl)amino, N,N—(C₁₋₄alkyl)₂amino, C₁₋₄alkanoylamino, C₁₋₄alkylS(O)ₐ wherein a is 0 to 2, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more R⁶;
R⁶ is selected from halo, nitro, cyano, trifluoromethyl, C₁₋₄alkyl, C₂₋₄alkenyl, C₁₋₄alkoxy, N,N—(C₁₋₄alkyl)₂amino, C₁₋₄alkylS(O)ₐ wherein a is 0 to 2, C₁₋₄alkoxycarbonylamino, carbocyclyl, heterocyclyl and carbocyclylC₀₋₄alkylene-Z-; wherein R⁶ may be optionally substituted on carbon by one or more R⁸;
R⁵ is selected from C₁₋₄alkyl, C₁₋₄alkanoyl and C₁₋₄alkoxycarbonyl;
Z is —S(O)ₐ—, —O—, —NR¹⁰—, —C(O)— or —OC(O)NR¹⁰—; wherein a is 0 to 2; wherein R¹⁰ is selected from hydrogen; and
R⁸ is selected from halo;
R¹² is hydroxy, methyl, ethyl or propyl;
m is 0 or 1; and
q is 0 or 1;
or a pharmaceutically acceptable salt thereof.

10. A compound selected from:
1-(3-fluoro-4-methoxybenzoyl)-4-(4-fluorobenzoyl)piperidine;
1-(quinoline-3-ylcarbonyl)-4-(4-fluorobenzoyl)piperidine;
1-(quinoline-2-ylcarbonyl)-4-(4-fluorobenzoyl)piperidine;
1-(5-trifluoromethylfur-2-yl)-4-(4-fluorobenzoyl)piperidine;
1-(3-trifluoromethoxybenzoyl)-4-(4-fluorobenzoyl)piperidine;
1-(tetrahydrofur-2-ylcarbonyl)-4-(4-chlorobenzoyl)piperidine;
1-(5-trifluoromethylfur-2-yl)-4-(4-chlorobenzoyl)piperidine;
1-(pyrid-2-ylcarbonyl)-4-(4-chlorobenzoyl)piperidine;
1-(thiazol-4-ylcarbonyl)-4-(4-chlorobenzoyl)piperidine;
1-(3,3,3-trifluoropropionyl)-4-(4-fluorobenzoyl)piperidine;
1-(4-fluorobenzoyl)-4-(3-mesylbenzoyl)piperidine;
or a pharmaceutically acceptable salt thereof.

11. A compound of formula (Ig): 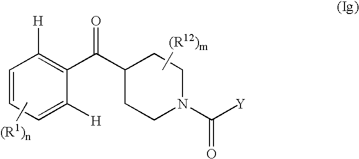
wherein:
R¹ is a substituent on carbon and is selected from halo, cyano, C₁₋₄alkyl, C₁₋₄alkoxy, C₁₋₄alkylS(O)₂, N—(C₁₋₄alkyl)sulphamoyl or N,N—(C₁₋₄alkyl)₂sulphamoyl; wherein R¹ may be optionally substituted on carbon by one or more groups selected from R³;
n is 0-3; wherein the values of R¹ may be the same or different;
Y is phenyl, pyrimidine, furan, thiophene or thiazole; wherein Y may be optionally substituted on carbon by one or more R²;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio or N,N—(C₁₋₄alkyl)₂aminothiocarbonylthio; wherein R² may be optionally substituted on carbon by one or more R⁶;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl or C₁₋₄alkylsulphonylamino; wherein R³ and R⁶ may be independently optionally substituted on carbon by one or more R⁸;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
R¹² is hydroxy, methyl, ethyl or propyl;
m is 0 or 1;
or a pharmaceutically acceptable salt thereof;
with the proviso that said compound is not 1,4-dibenzoylpiperidine;
4-hydroxy-1,4-dibenzoylpiperidine; 1-(3,4,5-trimethoxybenzoyl)-1-benzoylpiperidine;
1,4-di-(4-methylbenzoyl)piperidine; 1-(4-chlorobenzoyl)-4-benzoylpiperidine;
1-(3-nitrobenzoyl)-4-benzoylpiperidine;
1-(2-methoxy-4,6-ditrifluoromethylbenzoyl)-4-(4-chlorobenzoyl)piperidine;
1-(2,6-difluorobenzoyl)-4-benzoylpiperidine;
1-(3-trifluoromethylbenzoyl)-4-(benzoyl)piperidine;
1-(4-aminobenzoyl)-4-(4-fluorobenzoyl)piperidine;
1-(2-chloro-4-nitrobenzoyl)-4-benzoylpiperidine;
1-(4-methoxybenzoyl)-4-benzoylpiperidine; 1-(4-t-butylbenzoyl)-4-benzoylpiperidine;
1-(2,4-dihydroxybenzoyl)-4-(4-fluorobenzoyl)piperidine;
1-(4-nitrobenzoyl)-4-(4-fluorobenzoyl)piperidine;
1-(pyrid-3-ylcarbonyl)-4-(4-fluorobenzoyl)piperidine;
1-(thien-2-ylcarbonyl)-4-benzoylpiperidine;
1-(thien-2-ylcarbonyl)-4-(4-methylbenzoyl)piperidine; or
1-(fur-2-ylcarbonyl)-4-benzoylpiperidine.

12. A method for inhibiting 11βHSD1, comprising administering a compound of formula (Ih): 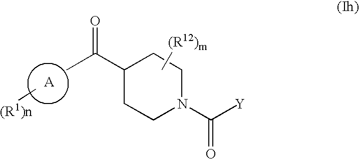
wherein:
Ring A is selected from carbocyclyl or heterocyclyl; wherein if said heterocyclyl contains an —NH— moiety that nitrogen may be optionally substituted by a group selected from R⁹;
R¹ is a substituent on carbon and is selected from halo, nitro, cyano, hydroxy, amino, carboxy, carbamoyl, mercapto, sulphamoyl, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R¹ may be optionally substituted on carbon by one or more R³; and wherein if said heterocyclyl contains an —NH— moiety that nitrogen may be optionally substituted by R⁴;
n is 0-5; wherein the values of R¹ may be the same or different;
Y is hydrogen, C₁₋₆alkyl, C₂₋₆alkenyl, C₂₋₆alkynyl, carbocyclyl or heterocyclyl; wherein Y may be optionally substituted on carbon by one or more R²; wherein if said heterocyclyl contains an —NH— moiety that nitrogen may be optionally substituted by R⁵;
R² is a substituent on carbon and is selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, aminothiocarbonylthio, N—(C₁₋₄alkyl)aminothiocarbonylthio, N,N—(C₁₋₄alkyl)₂aminothiocarbonylthio, carbocyclyl, heterocyclyl, carbocyclylC₀₋₄alkylene-Z- and heterocyclylC₀₋₄alkylene-Z-; wherein R² may be optionally substituted on carbon by one or more R⁶; and wherein if said heterocyclyl contains an —NH— moiety that nitrogen may be optionally substituted by R⁷;
R³ and R⁶ are independently selected from halo, nitro, cyano, hydroxy, amino, carboxy, carbamoyl, mercapto, sulphamoyl, trifluoromethyl, trifluoromethoxy, C₁₋₄alkyl, C₂₋₄alkenyl, C₂₋₄alkynyl, C₁₋₄alkoxy, C₁₋₄alkanoyl, C₁₋₄alkanoyloxy, N—(C₁₋₄alkyl)amino, N,N—(C₁₋₄alkyl)₂amino, C₁₋₄alkanoylamino, N—(C₁₋₄alkyl)carbamoyl, N,N—(C₁₋₄alkyl)₂carbamoyl, C₁₋₄alkylS(O)ₐ wherein a is 0 to 2, C₁₋₄alkoxycarbonyl, C₁₋₄alkoxycarbonylamino, C₁₋₄alkoxycarbonyl-N—(C₁₋₄alkyl)amino, N—(C₁₋₄alkyl)sulphamoyl, N,N—(C₁₋₄alkyl)₂sulphamoyl, C₁₋₄alkylsulphonylamino, carbocyclyl, heterocyclyl, carbocyclylC₀₋₄alkylene-Z- and heterocyclylC₀₋₄alkylene-Z-; wherein R³ and R⁶ may be independently optionally substituted on carbon by one or more R⁸; and wherein if said heterocyclyl contains an —NH— moiety that nitrogen may be optionally substituted by R¹³;
R⁴, R⁵, R⁷ R⁹ and R¹³ are independently selected from C₁₋₄alkyl, C₁₋₄alkanoyl, C₁₋₄alkylsulphonyl, C₁₋₄alkoxycarbonyl, carbamoyl, N—(C₁₋₄alkyl)carbamoyl, N,N—(C₁₋₄alkyl)₂carbamoyl, benzyl, benzyloxycarbonyl, benzoyl and phenylsulphonyl;
R⁸ is selected from halo, nitro, cyano, hydroxy, trifluoromethoxy, trifluoromethyl, amino, carboxy, carbamoyl, mercapto, sulphamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulphinyl, ethylsulphinyl, mesyl, ethylsulphonyl, methoxycarbonyl, ethoxycarbonyl, N-methylsulphamoyl, N-ethylsulphamoyl, N,N-dimethylsulphamoyl, N,N-diethylsulphamoyl or N-methyl-N-ethylsulphamoyl;
Z is —S(O)ₐ—, —O—, —NR¹⁰—, —C(O)—, —C(O)NR¹⁰—, —NR¹⁰C(O)—, —OC(O)NR¹⁰— or —SO₂NR¹⁰—; wherein a is 0 to 2; wherein R¹⁰ is selected from hydrogen and C₁₋₄alkyl;
R¹² is hydroxy, methyl, ethyl or propyl;
m is 0 or 1;
or a pharmaceutically acceptable salt thereof.

13. A pharmaceutical composition comprising a compound of claim 10 or 11, or a pharmaceutically acceptable salt thereof, in association with a pharmaceutically acceptable diluent or carrier.

14. (canceled)

15. (canceled)

16. A method for inhibiting 11βHSD1 in a warm-blooded animal, comprising administering a compound of claim 10 or 11, or a pharmaceutically acceptable salt thereof to a warm-blooded animal.

17. The method of claim 1 or 16 wherein inhibition of 11βHSD1 is associated with the treatment of metabolic syndrome.

18. The method of claim 1 or 16 wherein inhibition of 11βHSD1 is associated with the treatment of diabetes, obesity, hyperlipidaemia, hyperglycaemia, hyperinsulinemia or hypertension.

19. The method of claim 1 or 16 wherein inhibition of 11βHSD1 is associated with the treatment of glaucoma, osteoporosis, tuberculosis, dementia, cognitive disorders or depression.

20. (canceled)

21. The method of claim 18, wherein inhibition of 11βHSD1 is associated with the treatment of diabetes or obesity.
